SEQUENCE LISTING (117 sequences)

SEQ ID NO: 1 - protein, 7096 aa, Severe acute respiratory syndrome coronavirus 2 [sequence omitted]

SEQ ID NO: 2 - protein, 7073 aa, SARS coronavirus Tor2
MESLVLGVNEKTHVQLSLPVLQVRDVLVRGFGDSVEEALSEAREHLKNGTCGLVELEKGVLPQLEQPYVFIKRSDALSTNHGHKVVELVAEMDGIQYGRSGITLGVLVPHVGETPIAYRNVLLRKNGNKGAGGHSYGIDLKSYDLGDELGTDPIEDYEQNWNTKHGSGALRELTRELNGGAVTRYVDNNFCGPDGYPLDCIKDFLARAGKSMCTLSEQLDYIESKRGVYCCRDHEHEIAWFTERSDKSYEHQTPFEIKSAKKFDTFKGECPKFVFPLNSKVKVIQPRVEKKKTEGFMGRIRSVYPVASPQECNNMHLSTLMKCNHCDEVSWQTCDFLKATCEHCGTENLVIEGPTTCGYLPTNAVVKMPCPACQDPEIGPEHSVADYHNHSNIETRLRKGGRTRCFGGCVFAYVGCYNKRAYWVPRASADIGSGHTGITGDNVETLNEDLLEILSRERVNINIVGDFHLNEEVAIILASFSASTSAFIDTIKSLDYKSFKTIVESCGNYKVTKGKPVKGAWNIGQQRSVLTPLCGFPSQAAGVIRSIFARTLDAANHSIPDLQRAAVTILDGISEQSLRLVDAMVYTSDLLTNSVIIMAYVTGGLVQQTSQWLSNLLGTTVEKLRPIFEWIEAKLSAGVEFLKDAWEILKFLITGVFDIVKGQIQVASDNIKDCVKCFIDVVNKALEMCIDQVTIAGAKLRSLNLGEVFIAQSKGLYRQCIRGKEQLQLLMPLKAPKEVTFLEGDSHDTVLTSEEVVLKNGELEALETPVDSFTNGAIVGTPVCVNGLMLLEIKDKEQYCALSPGLLATNNVFRLKGGAPIKGVTFGEDTVWEVQGYKNVRITFELDERVDKVLNEKCSVYTVESGTEVTEFACVVAEAVVKTLQPVSDLLTNMGIDLDEWSVATFYLFDDAGEENFSSRMYCSFYPPDEEEEDDAECEEEEIDETCEHEYGTEDDYQGLPLEFGASAETVRVEEEEEEDWLDDTTEQSEIEPEPEPTPEEPVNQFTGYLKLTDNVAIKCVDIVKEAQSANPMVIVNAANIHLKHGGGVAGALNKATNGAMQKESDDYIKLNGPLTVGGSCLLSGHNLAKKCLHVVGPNLNAGEDIQLLKAAYENFNSQDILLAPLLSAGIFGAKPLQSLQVCVQTVRTQVYIAVNDKALYEQVVMDYLDNLKPRVEAPKQEEPPNTEDSKTEEKSVVQKPVDVKPKIKACIDEVTTTLEETKFLTNKLLLFADINGKLYHDSQNMLRGEDMSFLEKDAPYMVGDVITSGDITCVVIPSKKAGGTTEMLSRALKKVPVDEYITTYPGQGCAGYTLEEAKTALKKCKSAFYVLPSEAPNAKEEILGTVSWNLREMLAHAEETRKLMPICMDVRAIMATIQRKYKGIKIQEGIVDYGVRFFFYTSKEPVASIITKLNSLNEPLVTMPIGYVTHGFNLEEAARCMRSLKAPAVVSVSSPDAVTTYNGYLTSSSKTSEEHFVETVSLAGSYRDWSYSGQRTELGVEFLKRGDKIVYHTLESPVEFHLDGEVLSLDKLKSLLSLREVKTIKVFTTVDNTNLHTQLVDMSMTYGQQFGPTYLDGADVTKIKPHVNHEGKTFFVLPSDDTLRSEAFEYYHTLDESFLGRYMSALNHTKKWKFPQVGGLTSIKWADNNCYLSSVLLALQQLEVKFNAPALQEAYYRARAGDAANFCALILAYSNKTVGELGDVRETMTHLLQHANLESAKRVLNVVCKHCGQKTTTLTGVEAVMYMGTLSYDNLKTGVSIPCVCGRDATQYLVQQESSFVMMSAPPAEYKLQQGTFLCANEYTGNYQCGHYTHITAKETLYRIDGAHLTKMSEYKGPVTDVFYKETSYTTTIKPVSYKLDGVTYTEIEPKLDGYYKKDNAYYTEQPIDLVPTQPLPNASFDNFKLTCSNTKFADDLNQMTGFTKPASRELSVTFFPDLNGDVVAIDYRHYSASFKKGAKLLHKPIVWHINQATTKTTFKPNTWCLRCLWSTKPVDTSNSFEVLAVEDTQGMDNLACESQQPTSEEVVENPTIQKEVIECDVKTTEVVGNVILKPSDEGVKVTQELGHEDLMAAYVENTSITIKKPNELSLALGLKTIATHGIAAINSVPWSKILAYVKPFLGQAAITTSNCAKRLAQRVFNNYMPYVFTLLFQLCTFTKSTNSRIRASLPTTIAKNSVKSVAKLCLDAGINYVKSPKFSKLFTIAMWLLLLSICLGSLICVTAAFGVLLSNFGAPSYCNGVRELYLNSSNVTTMDFCEGSFPCSICLSGLDSLDSYPALETIQVTISSYKLDLTILGLAAEWVLAYMLFTKFFYLLGLSAIMQVFFGYFASHFISNSWLMWFIISIVQMAPVSAMVRMYIFFASFYYIWKSYVHIMDGCTSSTCMMCYKRNRATRVECTTIVNGMKRSFYVYANGGRGFCKTHNWNCLNCDTFCTGSTFISDEVARDLSLQFKRPINPTDQSSYIVDSVAVKNGALHLYFDKAGQKTYERHPLSHFVNLDNLRANNTKGSLPINVIVFDGKSKCDESASKSASVYYSQLMCQPILLLDQALVSDVGDSTEVSVKMFDAYVDTFSATFSVPMEKLKALVATAHSELAKGVALDGVLSTFVSAARQGVVDTDVDTKDVIECLKLSHHSDLEVTGDSCNNFMLTYNKVENMTPRDLGACIDCNARHINAQVAKSHNVSLIWNVKDYMSLSEQLRKQIRSAAKKNNIPFRLTCATTRQVVNVITTKISLKGGKIVSTCFKLMLKATLLCVLAALVCYIVMPVHTLSIHDGYTNEIIGYKAIQDGVTRDIISTDDCFANKHAGFDAWFSQRGGSYKNDKSCPVVAAIITREIGFIVPGLPGTVLRAINGDFLHFLPRVFSAVGNICYTPSKLIEYSDFATSACVLAAECTIFKDAMGKPVPYCYDTNLLEGSISYSELRPDTRYVLMDGSIIQFPNTYLEGSVRVVTTFDAEYCRHGTCERSEVGICLSTSGRWVLNNEHYRALSGVFCGVDAMNLIANIFTPLVQPVGALDVSASVVAGGIIAILVTCAAYYFMKFRRVFGEYNHVVAANALLFLMSFTILCLVPAYSFLPGVYSVFYLYLTFYFTNDVSFLAHLQWFAMFSPIVPFWITAIYVFCISLKHCHWFFNNYLRKRVMFNGVTFSTFEEAALCTFLLNKEMYLKLRSETLLPLTQYNRYLALYNKYKYFSGALDTTSYREAACCHLAKALNDFSNSGADVLYQPPQTSITSAVLQSGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQGKFKKIVKGTHHWMLLTFLTSLLILVQSTQWSLFFFVYENAFLPFTLGIMAIAACAMLLVKHKHAFLCLFLLPSLATVAYFNMVYMPASWVMRIMTWLELADTSLSGYRLKDCVMYASALVLLILMTARTVYDDAARRVWTLMNVITLVYKVYYGNALDQAISMWALVISVTSNYSGVVTTIMFLARAIVFVCVEYYPLLFITGNTLQCIMLVYCFLGYCCCCYFGLFCLLNRYFRLTLGVYDYLVSTQEFRYMNSQGLLPPKSSIDAFKLNIKLLGIGGKPCIKVATVQSKMSDVKCTSVVLLSVLQQLRVESSSKLWAQCVQLHNDILLAKDTTEAFEKMVSLLSVLLSMQGAVDINRLCEEMLDNRATLQAIASEFSSLPSYAAYATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVVPDYGTYKNTCDGNTFTYASALWEIQQVVDADSKIVQLSEINMDNSPNLAWPLIVTALRANSAVKLQNNELSPVALRQMSCAAGTTQTACTDDNALAYYNNSKGGRFVLALLSDHQDLKWARFPKSDGTGTIYTELEPPCRFVTDTPKGPKVKYLYFIKGLNNLNRGMVLGSLAATVRLQAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFFKRVCGVSAARLTPCGTGTSTDVVYRAFDIYNEKVAGFAKFLKTNCCRFQEKDEEGNLLDSYFVVKRHTMSNYQHEETIYNLVKDCPAVAVHDFFKFRVDGDMVPHISRQRLTKYTMADLVYALRHFDEGNCDTLKEILVTYNCCDDDYFNKKDWYDFVENPDILRVYANLGERVRQSLLKTVQFCDAMRDAGIVGVLTLDNQDLNGNWYDFGDFVQVAPGCGVPIVDSYYSLLMPILTLTRALAAESHMDADLAKPLIKWDLLKYDFTEERLCLFDRYFKYWDQTYHPNCINCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVETPHLMGWDYPKCDRAMPNMLRIMASLVLARKHNTCCNLSHRFYRLANECAQVLSEMVMCGGSLYVKPGGTSSGDATTAYANSVFNICQAVTANVNALLSTDGNKIADKYVRNLQHRLYECLYRNRDVDHEFVDEFYAYLRKHFSMMILSDDAVVCYNSNYAAQGLVASIKNFKAVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKIGILCIMSDRDLYDKLQFTSLEIPRRNVATLQAENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSDSPCESHGKQVVSDIDYVPLKSATCITRCNLGGAVCRHHANEYRQYLDAYNMMISAGFSLWIYKQFDTYNLWNTFTRLQSLENVAYNVVNKGHFDGHAGEAPVSIINNAVYTKVDGIDVEIFENKTTLPVNVAFELWAKRNIKPVPEIKILNNLGVDIAANTVIWDYKREAPAHVSTIGVCTMTDIAKKPTESACSSLTVLFDGRVEGQVDLFRNARNGVLITEGSVKGLTPSKGPAQASVNGVTLIGESVKTQFNYFKKVDGIIQQLPETYFTQSRDLEDFKPRSQMETDFLELAMDEFIQRYKLEGYAFEHIVYGDFSHGQLGGLHLMIGLAKRSQDSPLKLEDFIPMDSTVKNYFITDAQTGSSKCVCSVIDLLLDDFVEIIKSQDLSVISKVVKVTIDYAEISFMLWCKDGHVETFYPKLQASQAWQPGVAMPNLYKMQRMLLEKCDLQNYGENAVIPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPRTKHVTKENDSKEGFFTYLCGFIKQKLALGGSIAVKITEHSWNADLYKLMGHFSWWTAFVTNVNASSSEAFLIGANYLGKPKEQIDGYTMHANYIFWRNTNPIQLSSYSLFDMSKFPLKLRGTAVMSLKENQINDMIYSLLEKGRLIIRENNRVVVSSDILVNN

SEQ ID NO: 3 - protein, 7078 aa, Middle East respiratory syndrome-related coronavirus
MSFVAGVTAQGARGTYRAALNSEKHQDHVSLTVPLCGSGNLVEKLSPWFMDGENAYEVVKAMLLKKEPLLYVPIRLAGHTRHLPGPRVYLVERLIACENPFMVNQLAYSSSANGSLVGTTLQGKPIGMFFPYDIELVTGKQNILLRKYGRGGYHYTPFHYERDNTSCPEWMDDFEADPKGKYAQNLLKKLIGGDVTPVDQYMCGVDGKPISAYAFLMAKDGITKLADVEADVAARADDEGFITLKNNLYRLVWHVERKDVPYPKQSIFTINSVVQKDGVENTPPHYFTLGCKILTLTPRNKWSGVSDLSLKQKLLYTFYGKESLENPTYIYHSAFIECGSCGNDSWLTGNAIQGFACGCGASYTANDVEVQSSGMIKPNALLCATCPFAKGDSCSSNCKHSVAQLVSYLSERCNVIADSKSFTLIFGGVAYAYFGCEEGTMYFVPRAKSVVSRIGDSIFTGCTGSWNKVTQIANMFLEQTQHSLNFVGEFVVNDVVLAILSGTTTNVDKIRQLLKGVTLDKLRDYLADYDVAVTAGPFMDNAINVGGTGLQYAAITAPYVVLTGLGESFKKVATIPYKVCNSVKDTLAYYAHSVLYRVFPYDMDSGVSSFSELLFDCVDLSVASTYFLVRILQDKTGDFMSTIITSCQTAVSKLLDTCFEATEATFNFLLDLAGLFRIFLRNAYVYTSQGFVVVNGKVSTLVKQVLDLLNKGMQLLHTKVSWAGSKIIAVIYSGRESLIFPSGTYYCVTTKAKSVQQDLDVILPGEFSKKQLGLLQPTDNSTTVSVTVSSNMVETVVGQLEQTNMHSPDVIVGDYVIISEKLFVRSKEEDGFAFYPACTNGHAVPTLFRLKGGAPVKKVAFGGDQVHEVAAVRSVTVEYNIHAVLDTLLASSSLRTFVVDKSLSIEEFADVVKEQVSDLLVKLLRGMPIPDFDLDDFIDAPCYCFNAEGDASWSSTMIFSLHPVECDEECSEVEASDLEEGESECISETSTEQVDVSHETSDDEWAAAVDEAFPLDEAEDVTESVQEEAQPVEVPVEDIAQVVIADTLQETPVVPDTVEVPPQVVKLPSAPQTIQPEVKEVAPVYEADTEQTQNVTVKPKRLRKKRNVDPLSNFEHKVITECVTIVLGDAIQVAKCYGESVLVNAANTHLKHGGGIAGAINAASKGAVQKESDEYILAKGPLQVGDSVLLQGHSLAKNILHVVGPDARAKQDVSLLSKCYKAMNAYPLVVTPLVSAGIFGVKPAVSFDYLIREAKTRVLVVVNSQDVYKSLTIVDIPQSLTFSYDGLRGAIRKAKDYGFTVFVCTDNSANTKVLRNKGVDYTKKFLTVDGVQYYCYTSKDTLDDILQQANKSVGIISMPLGYVSHGLDLMQAGSVVRRVNVPYVCLLANKEQEAILMSEDVKLNPSEDFIKHVRTNGGYNSWHLVEGELLVQDLRLNKLLHWSDQTICYKDSVFYVVKNSTAFPFETLSACRAYLDSRTTQQLTIEVLVTVDGVNFRTVVLNNKNTYRSQLGCVFFNGADISDTIPDEKQNGHSLYLADNLTADETKALKELYGPVDPTFLHRFYSLKAAVHGWKMVVCDKVRSLKLSDNNCYLNAVIMTLDLLKDIKFVIPALQHAFMKHKGGDSTDFIALIMAYGNCTFGAPDDASRLLHTVLAKAELCCSARMVWREWCNVCGIKDVVLQGLKACCYVGVQTVEDLRARMTYVCQCGGERHRQLVEHTTPWLLLSGTPNEKLVTTSTAPDFVAFNVFQGIETAVGHYVHARLKGGLILKFDSGTVSKTSDWKCKVTDVLFPGQKYSSDCNVVRYSLDGNFRTEVDPDLSAFYVKDGKYFTSEPPVTYSPATILAGSVYTNSCLVSSDGQPGGDAISLSFNNLLGFDSSKPVTKKYTYSFLPKEDGDVLLAEFDTYDPIYKNGAMYKGKPILWVNKASYDTNLNKFNRASLRQIFDVAPIELENKFTPLSVESTPVEPPTVDVVALQQEMTIVKCKGLNKPFVKDNVSFVADDSGTPVVEYLSKEDLHTLYVDPKYQVIVLKDNVLSSMLRLHTVESGDINVVAASGSLTRKVKLLFRASFYFKEFATRTFTATTAVGSCIKSVVRHLGVTKGILTGCFSFAKMLFMLPLAYFSDSKLGTTEVKVSALKTAGVVTGNVVKQCCTAAVDLSMDKLRRVDWKSTLRLLLMLCTTMVLLSSVYHLYVFNQVLSSDVMFEDAQGLKKFYKEVRAYLGISSACDGLASAYRANSFDVPTFCANRSAMCNWCLISQDSITHYPALKMVQTHLSHYVLNIDWLWFAFETGLAYMLYTSAFNWLLLAGTLHYFFAQTSIFVDWRSYNYAVSSAFWLFTHIPMAGLVRMYNLLACLWLLRKFYQHVINGCKDTACLLCYKRNRLTRVEASTVVCGGKRTFYITANGGISFCRRHNWNCVDCDTAGVGNTFICEEVANDLTTALRRPINATDRSHYYVDSVTVKETVVQFNYRRDGQPFYERFPLCAFTNLDKLKFKEVCKTTTGIPEYNFIIYDSSDRGQESLARSACVYYSQVLCKSILLVDSSLVTSVGDSSEIATKMFDSFVNSFVSLYNVTRDKLEKLISTARDGVRRGDNFHSVLTTFIDAARGPAGVESDVETNEIVDSVQYAHKHDIQITNESYNNYVPSYVKPDSVSTSDLGSLIDCNAASVNQIVLRNSNGACIWNAAAYMKLSDALKRQIRIACRKCNLAFRLTTSKLRANDNILSVRFTANKIVGGAPTWFNALRDFTLKGYVLATIIVFLCAVLMYLCLPTFSMAPVEFYEDRILDFKVLDNGIIRDVNPDDKCFANKHRSFTQWYHEHVGGVYDNSITCPLTVAVIAGVAGARIPDVPTTLAWVNNQIIFFVSRVFANTGSVCYTPIDEIPYKSFSDSGCILPSECTMFRDAEGRMTPYCHDPTVLPGAFAYSQMRPHVRYDLYDGNMFIKFPEVVFESTLRITRTLSTQYCRFGSCEYAQEGVCITTNGSWAIFNDHHLNRPGVYCGSDFIDIVRRLAVSLFQPITYFQLTTSLVLGIGLCAFLTLLFYYINKVKRAFADYTQCAVIAVVAAVLNSLCICFVTSIPLCIVPYTALYYYATFYFTNEPAFIMHVSWYIMFGPIVPIWMTCVYTVAMCFRHFFWVLAYFSKKHVEVFTDGKLNCSFQDAASNIFVINKDTYAALRNSLTNDAYSRFLGLFNKYKYFSGAMETAAYREAAACHLAKALQTYSETGSDLLYQPPNCSITSGVLQSGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQSGVRKVTYGTAHWLFATLVSTYVIILQATKFTLWNYLFETIPTQLFPLLFVTMAFVMLLVKHKHTFLTLFLLPVAICLTYANIVYEPTTPISSALIAVANWLAPTNAYMRTTHTDIGVYISMSLVLVIVVKRLYNPSLSNFALALCSGVMWLYTYSIGEASSPIAYLVFVTTLTSDYTITVFVTVNLAKVCTYAIFAYSPQLTLVFPEVKMILLLYTCLGFMCTCYFGVFSLLNLKLRAPMGVYDFKVSTQEFRFMTANNLTAPRNSWEAMALNFKLIGIGGTPCIKVAAMQSKLTDLKCTSVVLLSVLQQLHLEANSRAWAFCVKCHNDILAATDPSEAFEKFVSLFATLMTFSGNVDLDALASDIFDTPSVLQATLSEFSHLATFAELEAAQKAYQEAMDSGDTSPQVLKALQKAVNIAKNAYEKDKAVARKLERMADQAMTSMYKQARAEDKKAKIVSAMQTMLFGMIKKLDNDVLNGIISNARNGCIPLSVIPLCASNKLRVVIPDFTVWNQVVTYPSLNYAGALWDITVINNVDNEIVKSSDVVDSNENLTWPLVLECTRASTSAVKLQNNEIKPSGLKTMVVSAGQEQTNCNTSSLAYYEPVQGRKMLMALLSDNAYLKWARVEGKDGFVSVELQPPCKFLIAGPKGPEIRYLYFVKNLNNLHRGQVLGHIAATVRLQAGSNTEFASNSSVLSLVNFTVDPQKAYLDFVNAGGAPLTNCVKMLTPKTGTGIAISVKPESTADQETYGGASVCLYCRAHIEHPDVSGVCKYKGKFVQIPAQCVRDPVGFCLSNTPCNVCQYWIGYGCNCDSLRQAALPQSKDSNFLNRVRGSIVNARIEPCSSGLSTDVVFRAFDICNYKAKVAGIGKYYKTNTCRFVELDDQGHHLDSYFVVKRHTMENYELEKHCYDLLRDCDAVAPHDFFIFDVDKVKTPHIVRQRLTEYTMMDLVYALRHFDQNSEVLKAILVKYGCCDVTYFENKLWFDFVENPSVIGVYHKLGERVRQAILNTVKFCDHMVKAGLVGVLTLDNQDLNGKWYDFGDFVITQPGSGVAIVDSYYSYLMPVLSMTDCLAAETHRDCDFNKPLIEWPLTEYDFTDYKVQLFEKYFKYWDQTYHANCVNCTDDRCVLHCANFNVLFAMTMPKTCFGPIVRKIFVDGVPFVVSCGYHYKELGLVMNMDVSLHRHRLSLKELMMYAADPAMHIASSNAFLDLRTSCFSVAALTTGLTFQTVRPGNFNQDFYDFVVSKGFFKEGSSVTLKHFFFAQDGNAAITDYNYYSYNLPTMCDIKQMLFCMEVVNKYFEIYDGGCLNASEVVVNNLDKSAGHPFNKFGKARVYYESMSYQEQDELFAMTKRNVIPTMTQMNLKYAISAKNRARTVAGVSILSTMTNRQYHQKMLKSMAATRGATCVIGTTKFYGGWDFMLKTLYKDVDNPHLMGWDYPKCDRAMPNMCRIFASLILARKHGTCCTTRDRFYRLANECAQVLSEYVLCGGGYYVKPGGTSSGDATTAYANSVFNILQATTANVSALMGANGNKIVDKEVKDMQFDLYVNVYRSTSPDPKFVDKYYAFLNKHFSMMILSDDGVVCYNSDYAAKGYIAGIQNFKETLYYQNNVFMSEAKCWVETDLKKGPHEFCSQHTLYIKDGDDGYFLPYPDPSRILSAGCFVDDIVKTDGTLMVERFVSLAIDAYPLTKHEDIEYQNVFWVYLQYIEKLYKDLTGHMLDSYSVMLCGDNSAKFWEEAFYRDLYSSPTTLQAVGSCVVCHSQTSLRCGTCIRRPFLCCKCCYDHVIATPHKMVLSVSPYVCNAPGCGVSDVTKLYLGGMSYFCVDHRPVCSFPLCANGLVFGLYKNMCTGSPSIVEFNRLATCDWTESGDYTLANTTTEPLKLFAAETLRATEEASKQSYAIATIKEIVGERQLLLVWEAGKSKPPLNRNYVFTGYHITKNSKVQLGEYIFERIDYSDAVSYKSSTTYKLTVGDIFVLTSHSVATLTAPTIVNQERYVKITGLYPTITVPEEFASHVANFQKSGYSKYVTVQGPPGTGKSHFAIGLAIYYPTARVVYTACSHAAVDALCEKAFKYLNIAKCSRIIPAKARVECYDRFKVNETNSQYLFSTINALPETSADILVVDEVSMCTNYDLSIINARIKAKHIVYVGDPAQLPAPRTLLTRGTLEPENFNSVTRLMCNLGPDIFLSMCYRCPKEIVSTVSALVYNNKLLAKKELSGQCFKILYKGNVTHDASSAINRPQLTFVKNFITANPAWSKAVFISPYNSQNAVSRSMLGLTTQTVDSSQGSEYQYVIFCQTADTAHANNINRFNVAITRAQKGILCVMTSQALFESLEFTELSFTNYKLQSQIVTGLFKDCSRETSGLSPAYAPTYVSVDDKYKTSDELCVNLNLPANVPYSRVISRMGFKLDATVPGYPKLFITREEAVRQVRSWIGFDVEGAHASRNACGTNVPLQLGFSTGVNFVVQPVGVVDTEWGNMLTGIAARPPPGEQFKHLVPLMHKGAAWPIVRRRIVQMLSDTLDKLSDYCTFVCWAHGFELTSASYFCKIGKEQKCCMCNRRAAAYSSPLQSYACWTHSCGYDYVYNPFFVDVQQWGYVGNLATNHDRYCSVHQGAHVASNDAIMTRCLAIHSCFIERVDWDIEYPYISHEKKLNSCCRIVERNVVRAALLAGSFDKVYDIGNPKGIPIVDDPVVDWHYFDAQPLTRKVQQLFYTEDMASRFADGLCLFWNCNVPKYPNNAIVCRFDTRVHSEFNLPGCDGGSLYVNKHAFHTPAYDVSAFRDLKPLPFFYYSTTPCEVHGNGSMIEDIDYVPLKSAVCITACNLGGAVCRKHATEYREYMEAYNLVSASGFRLWCYKTFDIYNLWSTFTKVQGLENIAFNVVKQGHFIGVEGELPVAVVNDKIFTKSGVNDICMFENKTTLPTNIAFELYAKRAVRSHPDFKLLHNLQADICYKFVLWDYERSNIYGTATIGVCKYTDIDVNSALNICFDIRDNCSLEKFMSTPNAIFISDRKIKKYPCMVGPDYAYFNGAIIRDSDVVKQPVKFYLYKKVNNEFIDPTECIYTQSRSCSDFLPLSDMEKDFLSFDSDVFIKKYGLENYAFEHVVYGDFSHTTLGGLHLLIGLYKKQQEGHIIMEEMLKGSSTIHNYFITETNTAAFKAVCSVIDLKLDDFVMILKSQDLGVVSKVVKVPIDLTMIEFMLWCKDGQVQTFYPRLQASADWKPGHAMPSLFKVQNVNLERCELANYKQSIPMPRGVHMNIAKYMQLCQYLNTCTLAVPANMRVIHFGAGSDKGIAPGTSVLRQWLPTDAIIIDNDLNEFVSDADITLFGDCVTVRVGQQVDLVISDMYDPTTKNVTGSNESKALFFTYLCNLINNNLALGGSVAIKITEHSWSVELYELMGKFAWWTVFCTNANASSSEGFLLGINYLGTIKENIDGGAMHANYIFWRNSTPMNLSTYSLFDLSKFQLKLKGTPVLQLKESQINELVISLLSQGKLLIRDNDTLSVSTDVLVNTYRKLR

SEQ ID NO: 4 - protein, 7217 aa, Human coronavirus HKU1
MIKTSKYGLGFKWAPEFRWLLPDAAEELASPMKSDEGGLCPSTGQAMESVGFVYDNHVKIDCRCILGQEWHVQSNLIRDIFVHEDLHVVEVLTKTAVKSGTAILIKSPLHSLGGFPKGYVMGLFRSYKTKRYVVHHLSMTTSTTNFGEDFLGWIVPFGFMPSYVHKWFQFCRLYIEESDLIISNFKFDDYDFSVEDVYAEVHAEPKGKYSQKAYALLRQYRGIKPVLFVDQYGCDYSGKLADCLQAYGHYSLQDMRQKQSVWLANCDFDIVVAWHVVRDSRFVMRLQTIATICGIKYVAQPTEDVVDGDVVIREPVHLLSADAIVLKLPSLMKVMTHMDDFSIKSIYNVDLCDCGFVMQYGYVDCFNDNCDFYGWVSGNMMDGFSCPLCCTVYDSSEVKAQSSGVIPENPVLFTNSTDTVNHDSFNLYGYSVTPFGSCIYWSPRPGLWIPIIKSSVKSYDDLVYSGVVGCKSIVKETALITHALYLDYVQCKCGNLEQNHILGVNNSWCRQLLLNRGDYNMLLKNIDLFVKRRADFACKFAVCGDGFVPFLLDGLIPRSYYLIQSGIFFTSLMSQFSQEVSDMCLKMCILFMDRVSVATFYIEHYVNRLVTQFKLLGTTLVNKMVNWFNTMLDASAPATGWLLYQLLNGLFVVSQANFNFVALIPDYAKILVNKFYTFFKLLLECVTVDVLKDMPVLKTINGLVCIVGNKFYNVSTGLIPGFVLPCNAQEQQIYFFEGVAESVIVEDDVIENVKSSLSSYEYCQPPKSVEKICIIDNMYMGKCGDKFFPIVMNDKNICLLDQAWRFPCAGRKVNFNEKPVVMEIPSLMTVKVMFDLDSTFDDILGKVCSEFEVEKGVTVDDFVAVVCDAIENALNSCKEHPVVGYQVRAFLNKLNDNVVYLFDEAGDEAMASRMYCTFAIEDVEDVISSEAVEDTIDGIVEDTINDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDVVTGDNDDEDNNDEEIVTGDNDDQIVVTGDDVDDIESIYDFDTYKALLVFNDVYNDALFVSYGSSVETETYFKVNGLWSPTITHTNCWLRSVLLVMQKLPFKFKDLAIENMWLSYKVGYNQSFVDYLLTTIPKAIVLPQGGFVADFAYWFLNQFDINAYANWCCLKCGFSFDLNGLDALFFYGDIVSHVCKCGHNMTLIAADLPCTLHFSLFDDNFCAFCTPKKIFIAACAVDVNVCHSVAVIGDEQIDGKFVTKFSGDKFDFIVGYGMSFSMSSFELAQLYGLCITPNVCFVKGDIINVARLVKADVIVNPANGHMLHGGGVAKAIAVAAGKKFSKETAAMVKSKGVCQVGDCYVSTGGKLCKTILNIVGPDARQDGRQSYVLLARAYKHLNNYDCCLSTLISAGIFSVPADVSLTYLLGVVDKQVILVSNNKEDFDIIQKCQITSVVGTKALAVRLTANVGRVIKFETDAYKLFLSGDDCFVSNSSVIQEVLLLRHDIQLNNDVRDYLLSKMTSLPKDWRLINKFDVINGVKTVKYFECPNSIYICSQGKDFGYVCDGSFYKATVNQVCVLLAKKIDVLLTVDGVNFKSISLTVGEVFGKILGNVFCDGIDVTKLKCSDFYADKILYQYENLSLADISAVQSSFGFDQQQLLAYYNFLTVCKWSVVVNGPFFSFEQSHNNCYVNVACLMLQHINLKFNKWQWQEAWYEFRAGRPHRLVALVLAKGHFKFDEPSDATDFIRVVLKQADLSGAICELELICDCGIKQESRVGVDAVMHFGTLAKTDLFNGYKIGCNCAGRIVHCTKLNVPFLICSNTPLSKDLPDDVVAANMFMGVGVGHYTHLKCGSPYQHYDACSVKKYTGVSGCLTDCLYLKNLTQTFTSMLTNYFLDDVEMVAYNPDLSQYYCDNGKYYTKPIIKAQFKPFAKVDGVYTNFKLVGHDICAQLNDKLGFNVDLPFVEYKVTVWPVATGDVVLASDDLYVKRYFKGCETFGKPVIWFCHDEASLNSLTYFNKPSFKSENRYSVLSVDSVSEESQGNVVTPVMESQISTKEVKLKGVRKTVKIEDAIIVNDENSSIKVVKSLSLVDVWDMYLTGCDYVVWVANELSRLVKSPTVREYIRYGIKPITIPIDLLCLRDDNQTLLVPKIFKARAIEFYGFLKWLFIYVFSLLHFTNDKTIFYTTEIASKFTFNLFCLALKNAFQTFRWSIFIKGFLVVATVFLFWFNFLYINVIFSDFYLPNISVFPIFVGRIVMWIKATFGLVTICDFYSKLGVGFTSHFCNGSFICELCHSGFDMLDTYAAIDFVQYEVDRRVLFDYVSLVKLIVELVIGYSLYTVWFYPLFCLIGLQLFTTWLPDLFMLETMHWLIRFIVFVANMLPAFVLLRFYIVVTAMYKVVGFIRHIVYGCNKAGCLFCYKRNCSVRVKCSTIVGGVIRYYDITANGGTGFCVKHQWNCFNCHSFKPGNTFITVEAAIELSKELKRPVNPTDASHYVVTDIKQVGCMMRLFYDRDGQRVYDDVDASLFVDINNLLHSKVKVVPNLYVVVVESDADRANFLNAVVFYAQSLYRPILLVDKKLITTACNGISVTQTMFDVYVDTFMSHFDVDRKSFNNFVNIAHASLREGVQLEKVLDTFVGCVRKCCSIDSDVETRFITKSMISAVAAGLEFTDENYNNLVPTYLKSDNIVAADLGVLIQNGAKHVQGNVAKAANISCIWFIDAFNQLTADLQHKLKKACVKTGLKLKLTFNKQEASVPILTTPFSLKGGVVLSNLLYILFFVSLICFILLWALLPTYSVYKSDIHLPAYASFKVIDNGVVRDISVNDLCFANKFFQFDQWYESTFGSVYYHNSMDCPIVVAVMDEDIGSTMFNVPTKVLRHGFHVLHFLTYAFASDSVQCYTPHIQISYNDFYASGCVLSSLCTMFKRGDGTPHPYCYSDGVMKNASLYTSLVPHTRYSLANSNGFIRFPDVISEGIVRIVRTRSMTYCRVGACEYAEEGICFNFNSSWVLNNDYYRSMPGTFCGRDLFDLFYQFFSSLIRPIDFFSLTASSIFGAILAIVVVLVFYYLIKLKRAFGDYTSVVVINVVVWCINFLMLFVFQVYPICACVYACFYFYVTLYFPSEISVIMHLQWIVMYGAIMPFWFCVTYVAMVIANHVLWLFSYCRKIGVNVCSDSTFEETSLTTFMITKDSYCRLKNSVSDVAYNRYLSLYNKYRYYSGKMDTAAYREAACSQLAKAMETFNHNNGNDVLYQPPTASVSTSFLQSGIVKMVSPTSKIEPCIVSVTYGSMTLNGLWLDDKVYCPRHVICSSSNMNEPDYSALLCRVTLGDFTIMSGRMSLTVVSYQMQGCQLVLTVSLQNPYTPKYTFGNVKPGETFTVLAAYNGRPQGAFHVTMRSSYTIKGSFLCGSCGSVGYVLTGDSVKFVYMHQLELSTGCHTGTDFTGNFYGPYRDAQVVQLPVKDYVQTVNVIAWLYAAILNNCAWFVQNDVCSTEDFNVWAMANGFSQVKADLVLDALASMTGVSIETLLAAIKRLYMGFQGRQILGSCTFEDELAPSDVYQQLAGVKLQSKTKRFIKETIYWILISTFLFSCIISAFVKWTIFMYINTHMIGVTLCVLCFVSFMMLLVKHKHFYLTMYIIPVLCTLFYVNYLVVYKEGFRGFTYVWLSYFVPAVNFTYVYEVFYGCILCVFAIFITMHSINHDIFSLMFLVGRIVTLISMWYFGSNLEEDVLLFITAFLGTYTWTTILSLAIAKIVANWLSVNIFYFTDVPYIKLILLSYLFIGYILSCYWGFFSLLNSVFRMPMGVYNYKISVQELRYMNANGLRPPRNSFEAILLNLKLLGIGGVPVIEVSQIQSKLTDVKCANVVLLNCLQHLHVASNSKLWQYCSVLHNEILSTSDLSVAFDKLAQLLIVLFANPAAVDTKCLASIDEVSDDYVQDSTVLQALQSEFVNMASFVEYEVAKKNLADAKNSGSVNQQQIKQLEKACNIAKSVYERDKAVARKLERMADLALTNMYKEARINDKKSKVVSALQTMLFSMVRKLDNQALNSILDNAVKGCVPLSAIPALAANTLTIIIPDKQVFDKVVDNVYVTYAGSVWHIQTVQDADGINKQLTDISVDSNWPLVIIANRYNEVANAVMQNNELMPHKLKIQVVNSGSDMNCNIPTQCYYNNGSSGRIVYAVLSDVDGLKYTKIMKDDGNCVVLELDPPCKFSIQDVKGLKIKYLYFIKGCNTLARGWVVGTLSSTIRLQAGVATEYAANSSILSLCAFSVDPKKTYLDYIQQGGVPIINCVKMLCDHAGTGMAITIKPEATINQDSYGGASVCIYCRARVEHPDVDGICKLRGKFVQVPLGIKDPILYVLTHDVCQVCGFWRDGSCSCVGSSVAVQSKDLNFLNRVRGTSVNARLVPCASGLSTDVQLRAFDICNTNRAGIGLYYKVNCCRFQRIDDDGNKLDKFFVVKRTNLEVYNKEKTYYELTKSCGVVAEHDFFTFDIDGSRVPHIVRRNLSKYTMLDLCYALRHFDRNDCSILCEILCEYADCKESYFSKKDWYDFVENPDIINIYKKLGPIFNRALLNTVIFADTLVEVGLVGVLTLDNQDLYGQWYDFGDFIQTAPGFGVAVADSYYSYMMPMLTMCHVLDCELFVNDSYRQFDLVQYDFTDYKLELFNKYFKYWGMKYHPNTVDCDNDRCIIHCANFNILFSMVLPNTCFGPLVRQIFVDGVPFVVSIGYHYKELGVVMNLDVDTHRYRLSLKDLLLYAADPAMHVASASALLDLRTCCFSVAAITSGIKFQTVKPGNFNQDFYEFVKSKGLFKEGSTVDLKHFFFTQDGNAAITDYNYYKYNLPTMVDIKQLLFVLEVVYKYFEIYDGGCIPASQVIVNNYDKSAGYPFNKFGKARLYYEALSFEEQNEIYAYTKRNVLPTLTQMNLKYAISAKNRARTVAGVSILSTMTGRMFHQKCLKSIAATRGVPVVIGTTKFYGGWDDMLRHLIKDVDNPVLMGWDYPKCDRAMPNILRIVSSLVLARKHEFCCSHGDRFYRLANECAQVLSEIVMCGGCYYVKPGGTSSGDATTAFANSVFNICQAVTANVCSLMACNGHKIEDLSIRNLQKRLYSNVYRTDYVDYTFVNEYYEFLCKHFSMMILSDDGVVCYNSDYASKGYIANISVFQQVLYYQNNVFMSESKCWVENDITNGPHEFCSQHTMLVKIDGDYVYLPYPDPSRILGAGCFVDDLLKTDSVLLIERFVSLAIDAYPLVYHENEEYQKVFRVYLEYIKKLYNDLGTQILDSYSVILSTCDGLKFTEESFYKNMYLKSAVMQSVGACVVCSSQTSLRCGSCIRKPLLCCKCCYDHVMATNHKYVLSVSPYVCNAPNCDVSDVTKLYLGGMSYYCENHKPHYSFKLVMNGMVFGLYKQSCTGSPYIDDFNKIASCKWTEVDDYVLANECIERLKLFAAETQKATEEAFKQSYASATIQEIVSDREVILCWETGKVKPPLNKNYVFTGYHFTSTGKTVLGEYVFDKSELTNGVYYRATTTYKLSIGDVFVLTSHSVASLSAPTLVPQENYASIRFSSVYSVPLVFQNNVANYQHIGMKRYCTVQGPPGTGKSHLAIGLAVYYYTARVVYTAASHAAVDALCEKAYKFLNINDCTRIIPAKVRVDCYDKFKINDTTCKYVFTTINALPELVTDIVVVDEVSMLTNYELSVINARIKAKHYVYIGDPAQLPAPRVLLSKGSLEPRHFNSITKIMCCLGPDIFLGNCYRCPKEIVETVSALVYDNKLKAKNDNSSLCFKVYFKGQTTHESSSAVNIQQIYLISKFLKANPVWNSAVFISPYNSQNYVAKRVLGVQTQTVDSAQGSEYDYVIYSQTAETAHSVNVNRFNVAITRAKKGIFCVMSNMQLFESLNFITLPLDKIQNQTLPRLHCTTNLFKDCSKSCLGYHPAHAPSFLAVDDKYKVNENLAVNLNICEPVLTYSRLISLMGFKLDLTLDGYSKLFITKDEAIKRVRGWVGFDVEGAHATRENIGTNFPLQIGFSTGVDFVVEATGLFAERDCYTFKKTVAKAPPGEKFKHLIPLMSKGQKWDIVRIRIVQMLSDYLLDLSDSVVFITWSASFELTCLRYFAKLGRELNCNVCSNRATCYNSRTGYYGCWRHSYTCDYVYNPLIVDIQQWGYTGSLTSNHDIICNVHKGAHVASADAIMTRCLAIYDCFCKSVNWNLEYPIISNEVSINTSCRLLQRVMLKAAMLCNRYNLCYDIGNPKGLACVKDYEFKFYDAFPVAKFVKQLFYVYDVHKDNFKDGLCMFWNCNVDKYPSNSIVCRFDTRVLNKLNLPGCNGGSLYVNKHAFHTNPFTRTVFENLKPMPFFYYSDTPCVYVDGLESKQVDYVPLRSATCITRCNLGGAVCSKHAEEYCNYLESYNIVTTAGFTFWVYKNFDFYNLWNTFTTLQSLENVIYNLVNVGHYDGRTGELPCAIINDKVVVKINNVDTVIFKNNTSFPTNIAVELFTKRSIRHHPELKILRNLNIDICWKHVLWDYVKDSLFCSSTYGVCKYTDLKFIENLNILFDGRDTGALEAFRKARNGVFISTEKLSRLSMIKGPQRADLNGVIVDKVGELKVEFWFAMRKDGDDVIFSRTDSLCSSHYWSPQGNLGGNCAGNVIGNDALTRFTIFTQSRVLSSFEPRSDLERDFIDMDDNLFIAKYGLEDYAFDHIVYGSFNHKVIGGLHLLIGLFRRLKKSNLLIQEFLQYDSSIHSYFITDQECGSSKSVCTVIDLLLDDFVSIVKSLNLSCVSKVVNINVDFKDFQFMLWCNDNKIMTFYPKMQATNDWKPGYSMPVLYKYLNVPLERVSLWNYGKPINLPTGCMMNVAKYTQLCQYLNTTTLAVPVNMRVLHLGAGSDKEVAPGSAVLRQWLPSGSILVDNDLNPFVSDSLVTYFGDCMTLPFDCHWDLIISDMYDPLTKNIGDYNVSKDGFFTYICHLIRDKLSLGGSVAIKITEFSWNADLYKLMSCFAFWTVFCTNVNASSSEGFLIGINYLGKSSFEIDGNVMHANYLFWRNSTTWNGGAYSLFDMTKFSLKLAGTAVVNLRPDQLNDLVYSLIERGKLLVRDTRKEIFVGDSLVNTC

SEQ ID NO: 5 - protein, 7095 aa, Human coronavirus OC43
MSKINKYGLELHWAPEFPWMFEDAEEKLDNPSSSEVDMICSTTAQKLETDGICPENHVMVDCRRLLKQECCVQSSLIREIVMNASPYDLEVLLQDALQSREAVLVTTPLGMSLEACYVRGCNPKGWTMGLFRRRSVCNTGRCTVNKHVAYQLYMIDPAGVCLGAGQFVGWVIPLAFMPVQSRKFIVPWVMYLRKRGEKGAYNKDHGRGGFGHVYDFKVEDAYDQVHDEPKGKFSKKAYALIRGYRGVKPLLYVDQYGCDYTGSLADGLEAYADKTLQEMKALFPTWSQELLFDVIVAWHVVRDPRYVMRLQSAATIRSVAYVANPTEDLCDGSVVIKEPVHVYADDSIILRQYNLVDIMSHFYMEADTVVNAFYGVALKDCGFVMQFGYIDCEQDSCDFKGWIPGNMIDGFACTTCGHVYEVGDLMAQSSGVLPVNPVLHTKSAAGYGGFGCKDSFTLYGQTVVYFGGCVYWSPARNIWIPILKSSVKSYDSLVYTGVLGCKAIVKETNLICKALYLDYVQHKCGNLHQRELLGVSDVWHKQLLLNRGVYKPLLENIDYFNMRRAKFSLETFTVCADGFMPFLLDDLVPRAYYLAVSGQAFCDYADKLCHAVVSKSKELLDVSLDSLGAAIHYLNSKIVDLAQHFSDFGTSFVSKIVHFFKTFTTSTALAFAWVLFHVLHGAYIVVESDIYFVKNIPRYASAVAQAFQSVAKVVLDSLRVTFIDGLSCFKIGRRRICLSGRKIYEVERGLLHSSQLPLDVYDLTMPSQVQKAKQKPIYLKGSGSDFSLADSVVEVVTTSLTPCGYSEPPKVAAKICIVDNVYMAKAGDKYYPVVVDDHVGLLDQAWRVPCAGRRVTFKEQPTVKEIISMPKIIKVFYELDNDFNTILNTACGVFEVDDTVDMEEFYAVVIDAIEEKLSPCKELEGVGAKVSAFLQKLEDNPLFLFDEAGEEVLAPKLYCAFTAPEDDDFLEESDVEEDDVEGEETDLTVTSAGQPCVASEQEESSEVLEDTLDDGPSVETSDSQVEEDVEMSDFVDLESVIQDYENVCFEFYTTEPEFVKVLGLYVPKATRNNCWLRSVLAVMQKLPCQFKDKNLQDLWVLYKQQYSQLFVDTLVNKIPANIVLPQGGYVADFAYWFLTLCDWQCVAYWKCIKCDLALKLKGLDAMFFYGDVVSHICKCGESMVLIDVDVPFTAHFALKDKLFCAFITKRIVYKAACVVDVNDSHSMAVVDGKQIDDHRITSITSDKFDFIIGHGMSFSMTTFEIAQLYGSCITPNVCFVKGDIIKVSKLVKAEVVVNPANGHMAHGGGVAKAIAVAAGQQFVKETTDMVKSKGVCATGDCYVSTGGKLCKTVLNVVGPDARTQGKQSYVLLERVYKHLNNYDCVVTTLISAGIFSVPSDVSLTYLLGTAKKQVVLVSNNQEDFDLISKCQITAVEGTKKLAARLSFNVGRSIVYETDANKLILINDVAFVSTFNVLQDVLSLRHDIALDDDARTFVQSNVDVVPEGWRVVNKFYQINGVRTVKYFECTGGIDICSQDKVFGYVQQGIFNKATVAQIKALFLDKVDILLTVDGVNFTNRFVPVGESFGKSLGNVFCDGVNVTKHKCDINYKGKVFFQFDNLSSEDLKAVRSSFNFDQKELLAYYNMLVNCFKWQVVVNGKYFTFKQANNNCFVNVSCLMLQSLHLTFKIVQWQEAWLEFRSGRPARFVALVLAKGGFKFGDPADSRDFLRVVFSQVDLTGAICDFEIACKCGVKQEQRTGLDAVMHFGTLSREDLEIGYTVDCSCGKKLIHCVRFDVPFLICSNTPASVKLPKGVGSANIFIGDKVGHYVHVKCEQSYQLYDASNVKKVTDVTGKLSDCLYLKNLKQTFKSVLTTYYLDDVKKIEYKPDLSQYYCDGGKYYTQRIIKAQFKTFEKVDGVYTNFKLIGHTVCDSLNAKLGFDSSKEFVEYKITEWPTATGDVVLATDDLYVKRYERGCITFGKPVIWLSHEKASLNSLTYFNRPSLVDDNKFDVLKVDDVDDGGDSSESGAKETKEINIIKLSGVKKPFKVEDSVIVNDDTSETKYVKSLSIVDVYDMWLTGCKYVVRTANALSRAVNVPTIRKFIKFGMTLVSIPIDLLNLREIKPAVNVVKAVRNKISVCFNFIKWLFVLLFGWIKISADNKVIYTTEIASKLTCKLVALAFKNAFLTFKWSMVARGACIIATIFLLWFNFIYANVIFSDFYLPKIGFLPTFVGKIAQWIKNTFSLVTICDLYSIQDVGFKNQYCNGSIACQFCLAGFDMLDNYKAIDVVQYEADRRAFVDYTGVLKIVIELIVSYALYTAWFYPLFALISIQILTTWLPELFMLSTLHWSFRLLVALANMLPAHVFMRFYIIIASFIKLFSLFRHVAYGCSKSGCLFCYKRNRSLRVKCSTIVGGMIRYYDVMANGGTGFCSKHQWNCIDCDSYKPGNTFITVEAALDLSKELKRPIQPTDVAYHTVTDVKQVGCSMRLFYDRDGQRTYDDVNASLFVDYSNLLHSKVKSVPNMHVVVVENDADKANFLNAAVFYAQSLFRPILMVDKNLITTANTGTSVTETMFDVYVDTFLSMFDVDKKSLNALIATAHSSIKQGTQIYKVLDTFLSCARKSCSIDSDVDTKCLADSVMSAVSAGLELTDESCNNLVPTYLKSDNIVAADLGVLIQNSAKHVQGNVAKIAGVSCIWSVDAFNQFSSDFQHKLKKACCKTGLKLKLTYNKQMANVSVLTTPFSLKGGAVFSYFVYVCFVLSLVCFIGLWCLMPTYTVHKSDFQLPVYASYKVLDNGVIRDVSVEDVCFANKFEQFDQWYESTFGLSYYSNSMACPIVVAVIDQDFGSTVFNVPTKVLRYGYHVLHFITHALSADGVQCYTPHSQISYSNFYASGCVLSSACTMFTMADGSPQPYCYTEGLMQNASLYSSLVPHVRYNLANAKGFIRFPEVLREGLVRIVRTRSMSYCRVGLCEEADEGICFNFNGSWVLNNDYYRSLPGTFCGRDVFDLIYQLFKGLAQPVDFLALTASSIAGAILAVIVVLVFYYLIKLKRAFGDYTSVVFVNVIVWCVNFMMLFVFQVYPILSCVYAICYFYATLYFPSEISVIMHLQWLVMYGTIMPLWFCLLYIAVVVSNHAFWVFSYCRKLGTSVRSDGTFEEMALTTFMITKDSYCKLKNSLSDVAFNRYLSLYNKYRYYSGKMDTAAYREAACSQLAKAMDTFTNNNGSDVLYQPPTASVSTSFLQSGIVKMVNPTSKVEPCVVSVTYGNMTLNGLWLDDKVYCPRHVICSASDMTNPDYTNLLCRVTSSDFTVLFDRLSLTVMSYQMRGCMLVLTVTLQNSRTPKYTFGVVKPGETFTVLAAYNGKPQGAFHVTMRSSYTIKGSFLCGSCGSVGYVIMGDCVKFVYMHQLELSTGCHTGTDFNGDFYGPYKDAQVVQLLIQDYIQSVNFVAWLYAAILNNCNWFVQSDKCSVEDFNVWALSNGFSQVKSDLVIDALASMTGVSLETLLAAIKRLKNGFQGRQIMGSCSFEDELTPSDVYQQLAGIKLQSKRTRLFKGTVCWIMASTFLFSCIITAFVKWTMFMYVTTNMFSITFCALCVISLAMLLVKHKHLYLTMYITPVLFTLLYNNYLVVYKHTFRGYVYAWLSYYVPSVEYTYTDEVIYGMLLLVGMVFVTLRSINHDLFSFIMFVGRLISVFSLWYKGSNLEEEILLMLASLFGTYTWTTVLSMAVAKVIAKWVAVNVLYFTDIPQIKIVLLCYLFIGYIISCYWGLFSLMNSLFRMPLGVYNYKISVQELRYMNANGLRPPKNSFEALMLNFKLLGIGGVPIIEVSQFQSKLTDVKCANVVLLNCLQHLHVASNSKLWHYCSTLHNEILATSDLSVAFEKLAQLLIVLFANPAAVDSKCLTSIEEVCDDYAKDNTVLQALQSEFVNMASFVEYEVAKKNLDEARFSGSANQQQLKQLEKACNIAKSAYERDRAVAKKLERMADLALTNMYKEARINDKKSKVVSALQTMLFSMVRKLDNQALNSILDNAVKGCVPLNAIPSLAANTLNIIVPDKSVYDQVVDNVYVTYAGNVWQIQTIQDSDGTNKQLNEISDDCNWPLVIIANRYNEVSATVLQNNELMPAKLKIQVVNSGPDQTCNTPTQCYYNNSNNGKIVYAILSDVDGLKYTKILKDDGNFVVLELDPPCKFTVQDAKGLKIKYLYFVKGCNTLARGWVVGTISSTVRLQAGTATEYASNSSILSLCAFSVDPKKTYLDFIQQGGTPIANCVKMLCDHAGTGMAITVKPDATTSQDSYGGASVCIYCRARVEHPDVDGLCKLRGKFVQVPVGIKDPVSYVLTHDVCRVCGFWRDGSCSCVSTDTTVQSKDTNFLNRVRGTSVDARLVPCASGLSTDVQLRAFDIYNASVAGIGLHLKVNCCRFQRVDENGDKLDQFFVVKRTDLTIYNREMKCYERVKDCKFVAEHDFFTFDVEGSRVPHIVRKDLTKYTMLDLCYALRHFDRNDCMLLCDILSIYAGCEQSYFTKKDWYDFVENPDIINVYKKLGPIFNRALVSATEFADKLVEVGLVGVLTLDNQDLNGKWYDFGDYVIAAPGCGVAIADSYYSYIMPMLTMCHALDCELYVNNAYRLFDLVQYDFTDYKLELFNKYFKHWSMPYHPNTVDCQDDRCIIHCANFNILFSMVLPNTCFGPLVRQIFVDGVPFVVSIGYHYKELGIVMNMDVDTHRYRLSLKDLLLYAADPALHVASASALYDLRTCCFSVAAITSGVKFQTVKPGNFNQDFYDFVLSKGLLKEGSSVDLKHFFFTQDGNAAITDYNYYKYNLPTMVDIKQLLFVLEVVYKYFEIYDGGCIPASQVIVNNYDKSAGYPFNKFGKARLYYEALSFEEQDEIYAYTKRNVLPTLTQMNLKYAISAKNRARTVAGVSILSTMTGRMFHQKCLKSIAATRGVPVVIGTTKFYGGWDDMLRRLIKDVDNPVLMGWDYPKCDRAMPNLLRIVSSLVLARKHETCCSQSDRFYRLANECAQVLSEIVMCGGCYYVKPGGTSSGDATTAFANSVFNICQAVSANVCALMSCNGNKIEDLSIRALQKRLYSHVYRSDKVDSTFVTEYYEFLNKHFSMMILSDDGVVCYNSDYASKGYIANISAFQQVLYYQNNVFMSESKCWVEHDINNGPHEFCSQHTMLVKMDGDDVYLPYPNPSRILGAGCFVDDLLKTDSVLLIERFVSLAIDAYPLVYHENEEYQKVFRVYLAYIKKLYNDLGNQILDSYSVILSTCDGQKFTDESFYKNMYLRSAVMQSVGACVVCSSQTSLRCGSCIRKPLLCCKCCYDHVMATDHKYVLSVSPYVCNAPGCDVNDVTKLYLGGMSYYCEDHKPQYSFKLVMNGLVFGLYKQSCTGSPYIDDFNRIASCKWTDVDDYILANECTERLKLFAAETQKATEEAFKQSYASATIQEIVSERELILSWEIGKVKPPLNKNYVFTGYHFTKNGKTVLGEYVFDKSELTNGVYYRATTTYKLSVGDVFVLTSHSVANLSAPTLVPQENYSSIRFASVYSVLETFQNNVVNYQHIGMKRYCTVQGPPGTGKSHLAIGLAVFYCTARVVYTAASHAAVDALCEKAYKFLNINDCTRIVPAKVRVECYDKFKINDTTRKYVFTTINALPEMVTDIVVVDEVSMLTNYELSVINARIRAKHYVYIGDPAQLPAPRVLLSKGTLEPKYFNTVTKLMCCLGPDIFLGTCYRCPKEIVDTVSALVYENKLKAKNESSSLCFKVYYKGVTTHESSSAVNMQQIYLINKFLKANPLWHKAVFISPYNSQNFAAKRVLGLQTQTVDSAQGSEYDYVIYSQTAETAHSVNVNRFNVAITRAKKGILCVMSNMQLFEALQFTTLTLDKVPQAVETKVQCSTNLFKDCSKSYSGYHPAHAPSFLAVDDKYKATGDLAVCLGIGDSAVTYSRLISLMGFKLDVTLDGYCKLFITKEEAVKRVRAWVGFDAEGAHATRDSIGTNFPLQLGFSTGIDFVVEATGLFADRDGYSFKKAVAKAPPGEQFKHLIPLMTRGHRWDVVRPRIVQMFADHLIDLSDCVVLVTWAANFELTCLRYFAKVGREISCNVCTKRATVYNSRTGYYGCWRHSVTCDYLYNPLIVDIQQWGYIGSLSSNHDLYCSVHKGAHVASSDAIMTRCLAVYDCFCNNINWNVEYPIISNELSINTSCRVLQRVILKAAMLCNRYTLCYDIGNPKAIACVKDFDFKFYDAQPIVKSVKTLLYSFEAHKDSFKDGLCMFWNCNVDKYPPNAVVCRFDTRVLNNLNLPGCNGGSLYVNKHAFHTKPFARAAFEHLKPMPFFYYSDTPCVYMDGMDAKQVDYVPLKSATCITRCNLGGAVCLKHAEEYREYLESYNTATTAGFTFWVYKTFDFYNLWNTFTKLQSLENVVYNLVKTGHYTGQAGEMPCAIINDKVVAKIDKEDVVIFINNTTYPTNVAVELFAKRSVRHHPELKLFRNLNIDVCWKHVIWDYARESIFCSNTYGVCMYTDLKFIDKLNVLFDGRDNGALEAFKRSNNGVYISTTKVKSLSMIRGPPRAELNGVVVDKVGDTDCVFYFAVRKEGQDVIFSQFDSLGVSSNQSPQGNLGSNGKPGNVGGNDALSISTIFTQSRVISSFTCRTDMEKDFIALDQDVFIQKYGLEDYAFEHIVYGNFNQKIIGGLHLLIGLYRRQQTSNLVVQEFVSYDSSIHSYFITDEKSGGSKSVCTVIDILLDDFVALVKSLNLNCVSKVVNVNVDFKDFQFMLWCNDEKVMTFYPRLQAASDWKPGYSMPVLYKYLNSPMERVSLWNYGKPVTLPTGCMMNVAKYTQLCQYLNTTTLAVPVNMRVLHLGAGSEKGVAPGSAVLRQWLPAGTILVDNDLYPFVSDSVATYFGDCITLPFDCQWDLIISDMYDPITKNIGEYNVSKDGFFTYICHMIRDKLALGGSVAIKITEFSWNAELYKLMGYFAFWTVFCTNANASSSEGFLIGINYLCKPKVEIDGNVMHANYLFWRNSTVWNGGAYSLFDMAKFPLKLAGTAVINLRADQINDMVYSLLEKGKLLIRDTNKEVFVGDSLVNVI

SEQ ID NO: 6 - protein, 6729 aa, Human coronavirus NL63
MFYNQVTLAVASDSEISGFGFAIPSVAVRTYSEAAAQGFQACRFVAFGLQDCVTGINDDDYVIALTGTNQLCAKILPFSDRPLNLRGWLIFSNSNYVLQDFDVVFGHGAGSVVFVDKYMCGFDGKPVLPKNMWEFRDYFNNNTDSIVIGGVTYQLAWDVIRKDLSYEQQNVLAIESIHYLGTTGHTLKSGCKLTNAKPPKYSSKVVLSGEWNAVYRAFGSPFITNGMSLLDIIVKPVFFNAFVKCNCGSESWSVGAWDGYLSSCCGTPAKKLCVVPGNVVPGDVIITSTSAGCGVKYYAGLVVKHITNITGVSLWRVTAVHSDGMFVASSSYDALLHRNSLDPFCFDVNTLLSNQLRLAFLGASVTEDVKFAASTGVIDISAGMFGLYDDILTNNKPWFVRKASGLFDAIWDAFVAAIKLVPTTTGVLVRFVKSIASTVLTVSNGVIIMCADVPDAFQSVYRTFTQAICAAFDFSLDVFKIGDVKFKRLGDYVLTENALVRLTTEVVRGVRDARIKKAMFTKVVVGPTTEVKFSVIELATVNLRLVDCAPVVCPKGKIVVIAGQAFFYSGGFYRFMVDPTTVLNDPVFTGDLFYTIKFSGFKLDGFNHQFVTASSATDAIIAVELLLLDFKTAVFVYTCVVDGCSVIVRRDATFATHVCFKDCYNVWEQFCIDNCGEPWFLTDYNAILQSNNPQCAIVQASESKVLLERFLPKCPEILLSIDDGHLWNLFVEKFNFVTDWLKTLKLTLTSNGLLGNCAKRFRRVLVKLLDVYNGFLETVCSVAYTAGVCIKYYAVNVPYVVISGFVSRVIRRERCDMTFPCVSCVTFFYEFLDTCFGVSKPNAIDVEHLELKETVFVEPKDGGQFFVSGDYLWYVVDDIYYPASCNGVLPVAFTKLAGGKISFSDDVIVHDVEPTHKVKLIFEFEDDVVTSLCKKSFGKSIIYTGDWEGLHEVLTSAMNVIGQHIKLPQFYIYDEEGGYDVSKPVMISQWPISNDSNGCVVEASTDFHQLECIVDDSVREEVDIIEQPFEEVEHVLSIKQPFSFSFRDELGVRVLDQSDNNCWISTTLVQLQLTKLLDDSIEMQLFKVGKVDSIVQKCYELSHLISGSLGDSGKLLSELLKEKYTCSITFEMSCDCGKKFDDQVGCLFWIMPYTKLFQKGECCICHKMQTYKLVSMKGTGVFVQDPAPIDIDAFPVKPICSSVYLGVKGSGHYQTNLYSFNKAIDGFGVFDIKNSSVNTVCFVDVDFHSVEIEAGEVKPFAVYKNVKFYLGDISHLVNCVSFDFVVNAANENLLHGGGVARAIDILTEGQLQSLSKDYISSNGPLKVGAGVMLECEKFNVFNVVGPRTGKHEHSLLVEAYNSILFENGIPLMPLLSCGIFGVRIENSLKALFSCDINKPLQVFVYSSNEEQAVLKFLDGLDLTPVIDDVDVVKPFRVEGNFSFFDCGVNALDGDIYLLFTNSILMLDKQGQLLDTKLNGILQQAALDYLATVKTVPAGNLVKLFVESCTIYMCVVPSINDLSFDKNLGRCVRKLNRLKTCVIANVPAIDVLKKLLSSLTLTVKFVVESNVMDVNDCFKNDNVVLKITEDGINVKDVVVESSKSLGKQLGVVSDGVDSFEGVLPINTDTVLSVAPEVDWVAFYGFEKAALFASLDVKPYGYPNDFVGGFRVLGTTDNNCWVNATCIILQYLKPTFKSKGLNVLWNKFVTGDVGPFVSFIYFITMSSKGQKGDAEEALSKLSEYLISDSIVTLEQYSTCDICKSTVVEVKSAIVCASVLKDGCDVGFCPHRHKLRSRVKFVNGRVVITNVGEPIISQPSKLLNGIAYTTFSGSFDNGHYVVYDAANNAVYDGARLFSSDLSTLAVTAIVVVGGCVTSNVPTIVSEKISVMDKLDTGAQKFFQFGDFVMNNIVLFLTWLLSMFSLLRTSIMKHDIKVIAKAPKRTGVILTRSFKYNIRSALFVIKQKWCVIVTLFKFLLLLYAIYALVFMIVQFSPFNSLLCGDIVSGYEKSTFNKDIYCGNSMVCKMCLFSYQEFNDLDHTSLVWKHIRDPILISLQPFVILVILLIFGNMYLRFGLLYFVAQFISTFGSFLGFHQKQWFLHFVPFDVLCNEFLATFIVCKIVLFVRHIIVGCNNADCVACSKSARLKRVPLQTIINGMHKSFYVNANGGTCFCNKHNFFCVNCDSFGPGNTFINGDIARELGNVVKTAVQPTAPAYVIIDKVDFVNGFYRLYSGDTFWRYDFDITESKYSCKEVLKNCNVLENFIVYNNSGSNITQIKNACVYFSQLLCEPIKLVNSELLSTLSVDFNGVLHKAYVDVLCNSFFKELTANMSMAECKATLGLTVSDDDFVSAVANAHRYDVLLSDLSFNNFFISYAKPEDKLSVYDIACCMRAGSKVVNHNVLIKESIPIVWGVKDFNTLSQEGKKYLVKTTKAKGLTFLLTFNDNQAITQVPATSIVAKQGAGFKRTYNFLWYVCLFVVALFIGVSFIDYTTTVTSFHGYDFKYIENGQLKVFEAPLHCVRNVFDNFNQWHEAKFGVVTTNSDKCPIVVGVSERINVVPGVPTNVYLVGKTLVFTLQAAFGNTGVCYDFDGVTTSDKCIFNSACTRLEGLGGDNVYCYNTDLIEGSKPYSTLQPNAYYKYDAKNYVRFPEILARGFGLRTIRTLATRYCRVGECRDSHKGVCFGFDKWYVNDGRVDDGYICGDGLIDLLVNVLSIFSSSFSVVAMSGHMLFNFLFAAFITFLCFLVTKFKRVFGDLSYGVFTVVCATLINNISYVVTQNLFFMLLYAILYFVFTRTVRYAWIWHIAYIVAYFLLIPWWLLTWFSFAAFLELLPNVFKLKISTQLFEGDKFIGTFESAAAGTFVLDMRSYERLINTISPEKLKNYAASYNKYKYYSGSASEADYRCACYAHLAKAMLDYAKDHNDMLYSPPTISYNSTLQSGLKKMAQPSGCVERCVVRVCYGSTVLNGVWLGDTVTCPRHVIAPSTTVLIDYDHAYSTMRLHNFSVSHNGVFLGVVGVTMHGSVLRIKVSQSNVHTPKHVFKTLKPGDSFNILACYEGIASGVFGVNLRTNFTIKGSFINGACGSPGYNVRNDGTVEFCYLHQIELGSGAHVGSDFTGSVYGNFDDQPSLQVESANLMLSDNVVAFLYAALLNGCRWWLCSTRVNVDGFNEWAMANGYTSVSSVECYSILAAKTGVSVEQLLASIQHLHEGFGGKNILGYSSLCDEFTLAEVVKQMYGVNLQSGKVIFGLKTMFLFSVFFTMFWAELFIYTNTIWINPVILTPIFCLLLFLSLVLTMFLKHKFLFLQVFLLPTVIATALYNCVLDYYIVKFLADHFNYNVSVLQMDVQGLVNVLVCLFVVFLHTWRFSKERFTHWFTYVCSLIAVAYTYFYSGDFLSLLVMFLCAISSDWYIGAIVFRLSRLIVFFSPESVFSVFGDVKLTLVVYLICGYLVCTYWGILYWFNRFFKCTMGVYDFKVSAAEFKYMVANGLHAPHGPFDALWLSFKLLGIGGDRCIKISTVQSKLTDLKCTNVVLLGCLSSMNIAANSSEWAYCVDLHNKINLCDDPEKAQSMLLALLAFFLSKHSDFGLDGLIDSYFDNSSTLQSVASSFVSMPSYIAYENARQAYEDAIANGSSSQLIKQLKRAMNIAKSEFDHEISVQKKINRMAEQAATQMYKEARSVNRKSKVISAMHSLLFGMLRRLDMSSVETVLNLARDGVVPLSVIPATSASKLTIVSPDLESYSKIVCDGSVHYAGVVWTLNDVKDNDGRPVHVKEITKENVETLTWPLILNCERVVKLQNNEIMPGKLKQKPMKAEGDGGVLGDGNALYNTEGGKTFMYAYISNKADLKFVKWEYEGGCNTIELDSPCRFMVETPNGPQVKYLYFVKNLNTLRRGAVLGFIGATIRLQAGKQTELAVNSGLLTACAFSVDPATTYLEAVKHGAKPVSNCIKMLSNGAGNGQAITTSVDANTNQDSYGGASICLYCRAHVPHPSMDGYCKFKGKCVQVPIGCLDPIRFCLENNVCNVCGCWLGHGCACDRTTIQSVDISYLNRARGSSAARLEPCNGTDIDKCVRAFDIYNKNVSFLGKCLKMNCVRFKNADLKDGYFVIKRCTKSVMEHEQSMYNLLNFSGALAEHDFFTWKDGRVIYGNVSRHNLTKYTMMDLVYAMRNFDEQNCDVLKEVLVLTGCCDNSYFDSKGWYDPVENEDIHRVYASLGKIVARAMLKCVALCDAMVAKGVVGVLTLDNQDLNGNFYDFGDFVVSLPNMGVPCCTSYYSYMMPIMGLTNCLASECFVKSDIFGSDFKTFDLLKYDFTEHKENLFNKYFKHWSFDYHPNCSDCYDDMCVIHCANFNTLFATTIPGTAFGPLCRKVFIDGVPLVTTAGYHFKQLGLVWNKDVNTHSVRLTITELLQFVTDPSLIIASSPALVDQRTICFSVAALSTGLTNQVVKPGHFNEEFYNFLRLRGFFDEGSELTLKHFFFAQNGDAAVKDFDFYRYNKPTILDICQARVTYKIVSRYFDIYEGGCIKACEVVVTNLNKSAGWPLNKFGKASLYYESISYEEQDALFALTKRNVLPTMTQLNLKYAISGKERARTVGGVSLLSTMTTRQYHQKHLKSIVNTRNATVVIGTTKFYGGWNNMLRTLIDGVENPMLMGWDYPKCDRALPNMIRMISAMVLGSKHVNCCTATDRFYRLGNELAQVLTEVVYSNGGFYFKPGGTTSGDASTAYANSIFNIFQAVSSNINRLLSVPSDSCNNVNVRDLQRRLYDNCYRLTSVEESFIDDYYGYLRKHFSMMILSDDGVVCYNKDYAELGYIADISAFKATLYYQNNVFMSTSKCWVEEDLTKGPHEFCSQHTMQIVDKDGTYYLPYPDPSRILSAGVFVDDVVKTDAVVLLERYVSLAIDAYPLSKHPNSEYRKVFYVLLDWVKHLNKNLNEGVLESFSVTLLDNQEDKFWCEDFYASMYENSTILQAAGLCVVCGSQTVLRCGDCLRKPMLCTKCAYDHVFGTDHKFILAITPYVCNASGCGVSDVKKLYLGGLNYYCTNHKPQLSFPLCSAGNIFGLYKNSATGSLDVEVFNRLATSDWTDVRDYKLANDVKDTLRLFAAETIKAKEESVKSSYAFATLKEVVGPKELLLSWESGKVKPPLNRNSVFTCFQISKDSKFQIGEFIFEKVEYGSDTVTYKSTVTTKLVPGMIFVLTSHNVQPLRAPTIANQEKYSSIYKLHPAFNVSDAYANLVPYYQLIGKQKITTIQGPPGSGKSHCSIGLGLYYPGARIVFVACAHAAVDSLCAKAMTVYSIDKCTRIIPARARVECYSGFKPNNTSAQYIFSTVNALPECNADIVVVDEVSMCTNYDLSVINQRLSYKHIVYVGDPQQLPAPRVMITKGVMEPVDYNVVTQRMCAIGPDVFLHKCYRCPAEIVNTVSELVYENKFVPVKPASKQCFKVFFKGNVQVDNGSSINRKQLEIVKLFLVKNPSWSKAVFISPYNSQNYVASRFLGLQIQTVDSSQGSEYDYVIYAQTSDTAHACNVNRFNVAITRAKKGIFCVMCDKTLFDSLKFFEIKHADLHSSQVCGLFKNCTRTPLNLPPTHAHTFLSLSDQFKTTGDLAVQIGSNNVCTYEHVISFMGFRFDISIPGSHSLFCTRDFAIRNVRGWLGMDVESAHVCGDNIGTNVPLQVGFSNGVNFVVQTEGCVSTNFGDVIKPVCAKSPPGEQFRHLIPLLRKGQPWLIVRRRIVQMISDYLSNLSDILVFVLWAGSLELTTMRYFVKIGPIKYCYCGNSATCYNSVSNEYCCFKHALGCDYVYNPYAFDIQQWGYVGSLSQNHHTFCNIHRNEHDASGDAVMTRCLAVHDCFVKNVDWTVTYPFIANEKFINGCGRNVQGHVVRAALKLYKPSVIHDIGNPKGVRCAVTDAKWYCYDKQPVNSNVKLLDYDYATHGQLDGLCLFWNCNVDMYPEFSIVCRFDTRTRSVFNLEGVNGGSLYVNKHAFHTPAYDKRAFVKLKPMPFFYFDDSDCDVVQEQVNYVPLRASSCVTRCNIGGAVCSKHANLYQKYVEAYNTFTQAGFNIWVPHSFDVYNLWQIFIETNLQSLENIAFNVVKKGCFTGVDGELPVAVVNDKVFVRYGDVDNLVFTNKTTLPTNVAFELFAKRKMGLTPPLSILKNLGVVATYKFVLWDYEAERPFTSYTKSVCKYTDFNEDVCVCFDNSIQGSYERFTLTTNAVLFSTVVIKNLTPIKLNFGMLNGMPVSSIKGDKGVEKLVNWYIYVRKNGQFQDHYDGFYTQGRNLSDFTPRSDMEYDFLNMDMGVFINKYGLEDFNFEHVVYGDVSKTTLGGLHLLISQFRLSKMGVLKADDFVTASDTTLRCCTVTYLNELSSKVVCTYMDLLLDDFVTILKSLDLGVISKVHEVIIDNKPYRWMLWCKDNHLSTFYPQLQSAEWKCGYAMPQIYKLQRMCLEPCNLYNYGAGIKLPSGIMLNVVKYTQLCQYLNSTTMCVPHNMRVLHYGAGSDKGVAPGTTVLKRWLPPDAIIIDNDINDYVSDADFSITGDCATVYLEDKFDLLISDMYDGRIKFCDGENVSKDGFFTYLNGVIREKLAIGGSVAIKITEYSWNKYLYELIQRFAFWTLFCTSVNTSSSEAFLIGINYLGDFIQGPFIAGNTVHANYIFWRNSTIMSLSYNSVLDLSKFECKHKATVVVTLKDSDVNDMVLSLIKSGRLLLRNNGRFGGFSNHLVSTK

SEQ ID NO: 7 - protein, 6758 aa, Human coronavirus 229E
MACNRVTLAVASDSEISANGCSTIAQAVRRYSEAASNGFRACRFVSLDLQDCIVGIADDTYVMGLHGNQTLFCNIMKFSDRPFMLHGWLVFSNSNYLLEEFDVVFGKRGGGNVTYTDQYLCGADGKPVMSEDLWQFVDHFGENEEIIINGHTYVCAWLTKRKPLDYKRQNNLAIEEIEYVHGDALHTLRNGSVLEMAKEVKTSSKVVLSDALDKLYKVFGSPVMTNGSNILEAFTKPVFISALVQCTCGTKSWSVGDWTGFKSSCCNVISNKLCVVPGNVKPGDAVITTQQAGAGIKYFCGMTLKFVANIEGVSVWRVIALQSVDCFVASSTFVEEEHVNRMDTFCFNVRNSVTDECRLAMLGAEMTSNVRRQVASGVIDISTGWFDVYDDIFAESKPWFVRKAEDIFGPCWSALASALKQLKVTTGELVRFVKSICNSAVAVVGGTIQILASVPEKFLNAFDVFVTAIQTVFDCAVETCTIAGKAFDKVFDYVLLDNALVKLVTTKLKGVRERGLNKVKYATVVVGSTEEVKSSRVERSTAVLTIANNYSKLFDEGYTVVIGDVAYFVSDGYFRLMASPNSVLTTAVYKPLFAFNVNVMGTRPEKFPTTVTCENLESAVLFVNDKITEFQLDYSIDVIDNEIIVKPNISLCVPLYVRDYVDKWDDFCRQYSNESWFEDDYRAFISVLDITDAAVKAAESKAFVDTIVPPCPSILKVIDGGKIWNGVIKNVNSVRDWLKSLKLNLTQQGLLGTCAKRFKRWLGILLEAYNAFLDTVVSTVKIGGLTFKTYAFDKPYIVIRDIVCKVENKTEAEWIELFPHNDRIKSFSTFESAYMPIADPTHFDIEEVELLDAEFVEPGCGGILAVIDEHVFYKKDGVYYPSNGTNILPVAFTKAAGGKVSFSDDVEVKDIEPVYRVKLCFEFEDEKLVDVCEKAIGKKIKHEGDWDSFCKTIQSALSVVSCYVNLPTYYIYDEEGGNDLSLPVMISEWPLSVQQAQQEATLPDIAEDVVDQVEEVNSIFDIETVDVKHDVSPFEMPFEELNGLKILKQLDNNCWVNSVMLQIQLTGILDGDYAMQFFKMGRVAKMIERCYTAEQCIRGAMGDVGLCMYRLLKDLHTGFMVMDYKCSCTSGRLEESGAVLFCTPTKKAFPYGTCLNCNAPRMCTIRQLQGTIIFVQQKPEPVNPVSFVVKPVCSSIFRGAVSCGHYQTNIYSQNLCVDGFGVNKIQPWTNDALNTICIKDADYNAKVEISVTPIKNTVDTTPKEEFVVKEKLNAFLVHDNVAFYQGDVDTVVNGVDFDFIVNAANENLAHGGGLAKALDVYTKGKLQRLSKEHIGLAGKVKVGTGVMVECDSLRIFNVVGPRKGKHERDLLIKAYNTINNEQGTPLTPILSCGIFGIKLETSLEVLLDVCNTKEVKVFVYTDTEVCKVKDFVSGLVNVQKVEQPKIEPKPVSVIKVAPKPYRVDGKFSYFTEDLLCVADDKPIVLFTDSMLTLDDRGLALDNALSGVLSAAIKDCVDINKAIPSGNLIKFDIGSVVVYMCVVPSEKDKHLDNNVQRCTRKLNRLMCDIVCTIPADYILPLVLSSLTCNVSFVGELKAAEAKVITIKVTEDGVNVHDVTVTTDKSFEQQVGVIADKDKDLSGAVPSDLNTSELLTKAIDVDWVEFYGFKDAVTFATVDHSAFAYESAVVNGIRVLKTSDNNCWVNAVCIALQYSKPHFISQGLDAAWNKFVLGDVEIFVAFVYYVARLMKGDKGDAEDTLTKLSKYLANEAQVQLEHYSSCVECDAKFKNSVASINSAIVCASVKRDGVQVGYCVHGIKYYSRVRSVRGRAIIVSVEQLEPCAQSRLLSGVAYTAFSGPVDKGHYTVYDTAKKSMYDGDRFVKHDLSLLSVTSVVMVGGYVAPVNTVKPKPVINQLDEKAQKFFDFGDFLIHNFVIFFTWLLSMFTLCKTAVTTGDVKIMAKAPQRTGVVLKRSLKYNLKASAAVLKSKWWLLAKFTKLLLLIYTLYSVVLLCVRFGPFNFCSETVNGYAKSNFVKDDYCDGSLGCKMCLFGYQELSQFSHLDVVWKHITDPLFSNMQPFIVMVLLLIFGDNYLRCFLLYFVAQMISTVGVFLGYKETNWFLHFIPFDVICDELLVTVIVIKVISFVRHVLFGCENPDCIACSKSARLKRFPVNTIVNGVQRSFYVNANGGSKFCKKHRFFCVDCDSYGYGSTFITPEVSRELGNITKTNVQPTGPAYVMIDKVEFENGFYRLYSCETFWRYNFDITESKYSCKEVFKNCNVLDDFIVFNNNGTNVTQVKNASVYFSQLLCRPIKLVDSELLSTLSVDFNGVLHKAYIDVLRNSFGKDLNANMSLAECKRALGLSISDHEFTSAISNAHRCDVLLSDLSFNNFVSSYAKPEEKLSAYDLACCMRAGAKVVNANVLTKDQTPIVWHAKDFNSLSAEGRKYIVKTSKAKGLTFLLTINENQAVTQIPATSIVAKQGAGDAGHSLTWLWLLCGLVCLIQFYLCFFMPYFMYDIVSSFEGYDFKYIENGQLKNFEAPLKCVRNVFENFEDWHYAKFGFTPLNKQSCPIVVGVSEIVNTVAGIPSNVYLVGKTLIFTLQAAFGNAGVCYDIFGVTTPEKCIFTSACTRLEGLGGNNVYCYNTALMEGSLPYSSIQANAYYKYDNGNFIKLPEVIAQGFGFRTVRTIATKYCRVGECVESNAGVCFGFDKWFVNDGRVANGYVCGTGLWNLVFNILSMFSSSFSVAAMSGQILLNCALGAFAIFCCFLVTKFRRMFGDLSVGVCTVVVAVLLNNVSYIVTQNLVTMIAYAILYFFATRSLRYAWIWCAAYLIAYISFAPWWLCAWYFLAMLTGLLPSLLKLKVSTNLFEGDKFVGTFESAAAGTFVIDMRSYEKLANSISPEKLKSYAASYNRYKYYSGNANEADYRCACYAYLAKAMLDFSRDHNDILYTPPTVSYGSTLQAGLRKMAQPSGFVEKCVVRVCYGNTVLNGLWLGDIVYCPRHVIASNTTSAIDYDHEYSIMRLHNFSIISGTAFLGVVGATMHGVTLKIKVSQTNMHTPRHSFRTLKSGEGFNILACYDGCAQGVFGVNMRTNWTIRGSFINGACGSPGYNLKNGEVEFVYMHQIELGSGSHVGSSFDGVMYGGFEDQPNLQVESANQMLTVNVVAFLYAAILNGCTWWLKGEKLFVEHYNEWAQANGFTAMNGEDAFSILAAKTGVCVERLLHAIQVLNNGFGGKQILGYSSLNDEFSINEVVKQMFGVNLQSGKTTSMFKSISLFAGFFVMFWAELFVYTTTIWVNPGFLTPFMILLVALSLCLTFVVKHKVLFLQVFLLPSIIVAAIQNCAWDYHVTKVLAEKFDYNVSVMQMDIQGFVNIFICLFVALLHTWRFAKERCTHWCTYLFSLIAVLYTALYSYDYVSLLVMLLCAISNEWYIGAIIFRICRFGVAFLPVEYVSYFDGVKTVLLFYMLLGFVSCMYYGLLYWINRFCKCTLGVYDFCVSPAEFKYMVANGLNAPNGPFDALFLSFKLMGIGGPRTIKVSTVQSKLTDLKCTNVVLMGILSNMNIASNSKEWAYCVEMHNKINLCDDPETAQELLLALLAFFLSKHSDFGLGDLVDSYFENDSILQSVASSFVGMPSFVAYETARQEYENAVANGSSPQIIKQLKKAMNVAKAEFDRESSVQKKINRMAEQAAAAMYKEARAVNRKSKVVSAMHSLLFGMLRRLDMSSVDTILNMARNGVVPLSVIPATSAARLVVVVPDHDSFVKMMVDGFVHYAGVVWTLQEVKDNDGKNVHLKDVTKENQEILVWPLILTCERVVKLQNNEIMPGKMKVKATKGEGDGGITSEGNALYNNEGGRAFMYAYVTTKPGMKYVKWEHDSGVVTVELEPPCRFVIDTPTGPQIKYLYFVKNLNNLRRGAVLGYIGATVRLQAGKQTEFVSNSHLLTHCSFAVDPAAAYLDAVKQGAKPVGNCVKMLTNGSGSGQAITCTIDSNTTQDTYGGASVCIYCRAHVAHPTMDGFCQYKGKWVQVPIGTNDPIRFCLENTVCKVCGCWLNHGCTCDRTAIQSFDNSYLNRVRGSSAARLEPCNGTDIDYCVRAFDVYNKDASFIGKNLKSNCVRFKNVDKDDAFYIVKRCIKSVMDHEQSMYNLLKGCNAVAKHDFFTWHEGRTIYGNVSRQDLTKYTMMDLCFALRNFDEKDCEVFKEILVLTGCCSTDYFEMKNWFDPIENEDIHRVYAALGKVVANAMLKCVAFCDEMVLKGVVGVLTLDNQDLNGNFYDFGDFVLCPPGMGIPYCTSYYSYMMPVMGMTNCLASECFMKSDIFGQDFKTFDLLKYDFTEHKEVLFNKYFKYWGQDYHPDCVDCHDEMCILHCSNFNTLFATTIPNTAFGPLCRKVFIDGVPVVATAGYHFKQLGLVWNKDVNTHSTRLTITELLQFVTDPTLIVASSPALVDKRTVCFSVAALSTGLTSQTVKPGHFNKEFYDFLRSQGFFDEGSELTLKHFFFTQKGDAAIKDFDYYRYNRPTMLDIGQARVAYQVAARYFDCYEGGCITSREVVVTNLNKSAGWPLNKFGKAGLYYESISYEEQDAIFSLTKRNILPTMTQLNLKYAISGKERARTVGGVSLLATMTTRQFHQKCLKSIVATRNATVVIGTTKFYGGWDNMLKNLMADVDDPKLMGWDYPKCDRAMPSMIRMLSAMILGSKHVTCCTASDKFYRLSNELAQVLTEVVYSNGGFYFKPGGTTSGDATTAYANSVFNIFQAVSSNINCVLSVNSSNCNNFNVKKLQRQLYDNCYRNSNVDESFVDDFYGYLQKHFSMMILSDDSVVCYNKTYAGLGYIADISAFKATLYYQNGVFMSTAKCWTEEDLSIGPHEFCSQHTMQIVDENGKYYLPYPDPSRIISAGVFVDDITKTDAVILLERYVSLAIDAYPLSKHPKPEYRKVFYALLDWVKHLNKTLNEGVLESFSVTLLDEHESKFWDESFYASMYEKSTVLQAAGLCVVCGSQTVLRCGDCLRRPMLCTKCAYDHVFGTDHKFILAITPYVCNTSGCNVNDVTKLYLGGLNYYCVDHKPHLSFPLCSAGNVFGLYKSSALGSMDIDVFNKLSTSDWSDIRDYKLANDAKESLRLFAAETVKAKEESVKSSYAYATLKEIVGPKELLLLWESGKAKPPLNRNSVFTCFQITKDSKFQVGEFVFEKVDYGSDTVTYKSTATTKLVPGMLFILTSHNVAPLRAPTMANQEKYSTIYKLHPSFNVSDAYANLVPYYQLIGKQRITTIQGPPGSGKSHCSIGIGVYYPGARIVFTACSHAAVDSLCAKAVTAYSVDKCTRIIPARARVECYSGFKPNNNSAQYVFSTVNALPEVNADIVVVDEVSMCTNYDLSVINQRISYKHIVYVGDPQQLPAPRVLISKGVMEPIDYNVVTQRMCAIGPDVFLHKCYRCPAEIVNTVSELVYENKFVPVKEASKQCFKIFERGSVQVDNGSSINRRQLDVVKRFIHKNSTWSKAVFISPYNSQNYVAARLLGLQTQTVDSAQGSEYDYVIFAQTSDTAHACNANRFNVAITRAKKGIFCIMSDRTLFDALKFFEITMTDLQSESSCGLFKDCARNPIDLPPSHATTYLSLSDRFKTSGDLAVQIGNNNVCTYEHVISYMGFRFDVSMPGSHSLFCTRDFAMRHVRGWLGMDVEGAHVTGDNVGTNVPLQVGFSNGVDFVAQPEGCVLTNTGSVVKPVRARAPPGEQFTHIVPLLRKGQPWSVLRKRIVQMIADFLAGSSDVLVFVLWAGGLELTTMRYFVKIGAVKHCQCGTVATCYNSVSNDYCCFKHALGCDYVYNPYVIDIQQWGYVGSLSTNHHAICNVHRNEHVASGDAIMTRCLAVYDCFVKNVDWSITYPMIANENAINKGGRTVQSHIMRAAIKLYNPKAIHDIGNPKGIRCAVTDAKWYCYDKNPINSNVKTLEYDYMTHGQMDGLCLFWNCNVDMYPEFSIVCRFDTRTRSTLNLEGVNGGSLYVNNHAFHTPAYDKRAMAKLKPAPFFYYDDGSCEVVHDQVNYVPLRATNCITKCNIGGAVCSKHANLYRAYVESYNIFTQAGFNIWVPTTFDCYNLWQTFTEVNLQGLENIAFNVVNKGSFVGADGELPVAISGDKVFVRDGNTDNLVFVNKTSLPTNIAFELFAKRKVGLTPPLSILKNLGVVATYKFVLWDYEAERPLTSFTKSVCGYTDFAEDVCTCYDNSIQGSYERFTLSTNAVLFSATAVKTGGKSLPAIKLNFGMLNGNAIATVKSEDGNIKNINWFVYVRKDGKPVDHYDGFYTQGRNLQDFLPRSTMEEDFLNMDIGVFIQKYGLEDFNFEHVVYGDVSKTTLGGLHLLISQVRLSKMGILKAEEFVAASDITLKCCTVTYLNDPSSKTVCTYMDLLLDDFVSVLKSLDLTVVSKVHEVIIDNKPWRWMLWCKDNAVATFYPQLQSAEWKCGYSMPGIYKTQRMCLEPCNLYNYGAGLKLPSGIMFNVVKYTQLCQYFNSTTLCVPHNMRVLHLGAGSDYGVAPGTAVLKRWLPHDAIVVDNDVVDYVSDADFSVTGDCATVYLEDKFDLLISDMYDGRTKAIDGENVSKEGFFTYINGFICEKLAIGGSIAIKVTEYSWNKKLYELVQRFSFWTMFCTSVNTSSSEAFVVGINYLGDFAQGPFIDGNIIHANYVFWRNSTVMSLSYNSVLDLSKFNCKHKATVVVQLKDSDINEMVLSLVRSGKLLVRGNGKCLSFSNHLVSTK

SEQ ID NO: 8 - protein, 32 aa, Severe acute respiratory syndrome-related coronavirus
DAVDCSQNPLAELKCSVKSFEIDKGIYQTSNF

SEQ ID NO: 9 - protein, 31 aa, Severe acute respiratory syndrome coronavirus 2
DAVDCALDPLSETKCTLKSFTVEKGIYQTSN

SEQ ID NO: 10 - protein, 76 aa, Severe acute respiratory syndrome-related coronavirus
VCGPKLSTDLIKNQCVNFNFNGLTGTGVLTPSSKRFQPFQQFGRDVSDFTDSVRDPKTSEILDISPCSFGGVSVIT

SEQ ID NO: 11 - protein, 75 aa, Severe acute respiratory syndrome coronavirus 2
VCGPKKSTNLVKNKCVNFNFNGLTGTGVLTESNKKFLPFQQFGRDIADTTDAVRDPQTLEILDITPCSFGGVSVI

SEQ ID NO: 12 - protein, 41 aa, Severe acute respiratory syndrome-related coronavirus
GTNASSEVAVLYQDVNCTDVSTAIHADQLTPAWRIYSTGNN

SEQ ID NO: 13 - protein, 40 aa, Severe acute respiratory syndrome coronavirus 2
GTNTSNQVAVLYQDVNCTEVPVAIHADQLTPTWRVYSTGS

SEQ ID NO: 14 - protein, 19 aa, Severe acute respiratory syndrome-related coronavirus
FSQILPDPLKPTKRSFIED

SEQ ID NO: 15 - protein, 18 aa, Severe acute respiratory syndrome coronavirus 2
FSQILPDPSKPSKRSFIE

SEQ ID NO: 16 - protein, 23 aa, Severe acute respiratory syndrome-related coronavirus
FGAGAALQIPFAMQMAYRFNGIG

SEQ ID NO: 17 - protein, 22 aa, Severe acute respiratory syndrome coronavirus 2
FGAGAALQIPFAMQMAYRFNGI

SEQ ID NO: 18 - protein, 24 aa, Severe acute respiratory syndrome-related coronavirus
MADNGTITVEELKQLLEQWNLVIG

SEQ ID NO: 19 - protein, 24 aa, Severe acute respiratory syndrome coronavirus 2
MADSNGTITVEELKKLLEQWNLVI

SEQ ID NO: 20 - protein, 21 aa, Severe acute respiratory syndrome-related coronavirus
PLMESELVIGAVIIRGHLRMA

SEQ ID NO: 21 - protein, 20 aa, Severe acute respiratory syndrome coronavirus 2
PLLESELVIGAVILRGHLRI

SEQ ID NO: 22 - protein, 22 aa, Severe acute respiratory syndrome-related coronavirus
PQGLPNNTASWFTALTQHGKEE

SEQ ID NO: 23 - protein, 21 aa, Severe acute respiratory syndrome coronavirus 2
RPQGLPNNTASWFTALTQHGK

SEQ ID NO: 24 - protein, 21 aa, Severe acute respiratory syndrome-related coronavirus
NNAATVLQLPQGTTLPKGFYA

SEQ ID NO: 25 - protein, 20 aa, Severe acute respiratory syndrome coronavirus 2
NNNAATVLQLPQGTTLPKGF

SEQ ID NO: 26 - protein, 48 aa, Severe acute respiratory syndrome-related coronavirus
KHIDAYKTFPPTEPKKDKKKKTDEAQPLPQRQKKQPTVTLLPAADMDD

SEQ ID NO: 27 - protein, 47 aa, Severe acute respiratory syndrome coronavirus 2
NKHIDAYKTFPPTEPKKDKKKKTDEAQPLPQRQKKQPTVTLLPAADM

SEQ ID NO: 28 - protein, 18 aa, Severe acute respiratory syndrome-related coronavirus
VRGWVFGSTMNNKSQSVI

SEQ ID NO: 29 - protein, 18 aa, Severe acute respiratory syndrome coronavirus 2
IRGWIFGTTLDSKTQSLL

SEQ ID NO: 30 - protein, 13 aa, Severe acute respiratory syndrome-related coronavirus
CTFEYISDAFSLD

SEQ ID NO: 31 - protein, 13 aa, Severe acute respiratory syndrome coronavirus 2
CTFEYVSQPFLMD

SEQ ID NO: 32 - protein, 13 aa, Severe acute respiratory syndrome-related coronavirus
DAFSLDVSEKSGN

SEQ ID NO: 33 - protein, 13 aa, Severe acute respiratory syndrome coronavirus 2
QPFLMDLEGKQGN

SEQ ID NO: 34 - protein, 17 aa, Severe acute respiratory syndrome-related coronavirus
TNFRAILTAFSPAQDIW

SEQ ID NO: 35 - protein, 23 aa, Severe acute respiratory syndrome coronavirus 2
TRFQTLLALHRSYLTPGDSSSGW

SEQ ID NO: 36 - protein, 18 aa, Severe acute respiratory syndrome-related coronavirus
KSFEIDKGIYQTSNFRVV

SEQ ID NO: 37 - protein, 18 aa, Severe acute respiratory syndrome coronavirus 2
KSFTVEKGIYQTSNFRVQ

SEQ ID NO: 38 - protein, 17 aa, Severe acute respiratory syndrome-related coronavirus
STFFSTFKCYGVSATKL

SEQ ID NO: 39 - protein, 17 aa, Severe acute respiratory syndrome coronavirus 2
SASFSTFKCYGVSPTKL

SEQ ID NO: 40 - protein, 10 aa, Severe acute respiratory syndrome-related coronavirus
KLPDDFMGCV

SEQ ID NO: 41 - protein, 10 aa, Severe acute respiratory syndrome coronavirus 2
KLPDDFTGCV

SEQ ID NO: 42 - protein, 18 aa, Severe acute respiratory syndrome-related coronavirus
NIDATSTGNYNYKYRYLR

SEQ ID NO: 43 - protein, 18 aa, Severe acute respiratory syndrome coronavirus 2
NLDSKVGGNYNYLYRLFR

SEQ ID NO: 44 - protein, 18 aa, Severe acute respiratory syndrome-related coronavirus
YLRHGKLRPFERDISNVP

SEQ ID NO: 45 - protein, 18 aa, Severe acute respiratory syndrome coronavirus 2
YLYRLFRKSNLKPFERDI

SEQ ID NO: 46 - protein, 13 aa, Severe acute respiratory syndrome-related coronavirus
RPFERDISNVPFS

SEQ ID NO: 47 - protein, 13 aa, Severe acute respiratory syndrome coronavirus 2
KPFERDISTEIYQ

SEQ ID NO: 48 - protein, 18 aa, Severe acute respiratory syndrome-related coronavirus
KSIVAYTMSLGADSSIAY

SEQ ID NO: 49 - protein, 18 aa, Severe acute respiratory syndrome coronavirus 2
QSIIAYTMSLGAENSVAY

SEQ ID NO: 50 - protein, 9 aa, Severe acute respiratory syndrome-related coronavirus
SIVAYTMSL

SEQ ID NO: 51 - protein, 9 aa, Severe acute respiratory syndrome coronavirus 2
SIIAYTMSL

SEQ ID NO: 52 - protein, 17 aa, Severe acute respiratory syndrome-related coronavirus
TECANLLLQYGSFCTQL

SEQ ID NO: 53 - protein, 17 aa, Severe acute respiratory syndrome coronavirus 2
TECSNLLLQYGSFCTQL

SEQ ID NO: 54 - protein, 18 aa, Severe acute respiratory syndrome-related coronavirus
VKQMYKTPTLKYFGGFNF

SEQ ID NO: 55 - protein, 18 aa, Severe acute respiratory syndrome coronavirus 2
VKQIYKTPPIKDFGGFNF

SEQ ID NO: 56 - protein, 17 aa, Severe acute respiratory syndrome-related coronavirus
ESLTTTSTALGKLQDVV

SEQ ID NO: 57 - protein, 17 aa, Severe acute respiratory syndrome coronavirus 2
DSLSSTASALGKLQDVV

SEQ ID NO: 58 - protein, 9 aa, Severe acute respiratory syndrome-related coronavirus
ALNTLVKQL

SEQ ID NO: 59 - protein, 9 aa, Severe acute respiratory syndrome coronavirus 2
ALNTLVKQL

SEQ ID NO: 60 - protein, 9 aa, Severe acute respiratory syndrome-related coronavirus
VLNDILSRL

SEQ ID NO: 61 - protein, 9 aa, Severe acute respiratory syndrome coronavirus 2
VLNDILSRL

SEQ ID NO: 62 - protein, 9 aa, Severe acute respiratory syndrome-related coronavirus
LITGRLQSL

SEQ ID NO: 63 - protein, 9 aa, Severe acute respiratory syndrome coronavirus 2
LITGRLQSL

SEQ ID NO: 64 - protein, 18 aa, Severe acute respiratory syndrome-related coronavirus
QLIRAAEIRASANLAATK

SEQ ID NO: 65 - protein, 18 aa, Severe acute respiratory syndrome coronavirus 2
QLIRAAEIRASANLAATK

SEQ ID NO: 66 - protein, 15 aa, Severe acute respiratory syndrome-related coronavirus
SWFITQRNFFSPQII

SEQ ID NO: 67 - protein, 15 aa, Severe acute respiratory syndrome coronavirus 2
HWFVTQRNFYEPQII

SEQ ID NO: 68 - protein, 9 aa, Severe acute respiratory syndrome-related coronavirus
RLNEVAKNL

SEQ ID NO: 69 - protein, 9 aa, Severe acute respiratory syndrome coronavirus 2
RLNEVAKNL

SEQ ID NO: 70 - protein, 9 aa, Severe acute respiratory syndrome-related coronavirus
NLNESLIDL

SEQ ID NO: 71 - protein, 9 aa, Severe acute respiratory syndrome coronavirus 2
NLNESLIDL

SEQ ID NO: 72 - protein, 9 aa, Severe acute respiratory syndrome-related coronavirus
FIAGLIAIV

SEQ ID NO: 73 - protein, 9 aa, Severe acute respiratory syndrome coronavirus 2
FIAGLIAIV

SEQ ID NO: 74 - protein, 15 aa, Severe acute respiratory syndrome-related coronavirus
RFFTLGSITAQPVKI

SEQ ID NO: 75 - protein, 15 aa, Severe acute respiratory syndrome coronavirus 2
RIFTIGTVTLKQGEI

SEQ ID NO: 76 - protein, 9 aa, Severe acute respiratory syndrome-related coronavirus
SITAQPVKI

SEQ ID NO: 77 - protein, 9 aa, Severe acute respiratory syndrome coronavirus 2
TVTLKQGEI

SEQ ID NO: 78 - protein, 10 aa, Severe acute respiratory syndrome-related coronavirus
TLACFVLAAV

SEQ ID NO: 79 - protein, 10 aa, Severe acute respiratory syndrome coronavirus 2
TLACFVLAAV

SEQ ID NO: 80 - protein, 9 aa, Severe acute respiratory syndrome-related coronavirus
GLMWLSYFV

SEQ ID NO: 81 - protein, 9 aa, Severe acute respiratory syndrome coronavirus 2
GLMWLSYFI

SEQ ID NO: 82 - protein, 9 aa, Severe acute respiratory syndrome-related coronavirus
HLRMAGHSL

SEQ ID NO: 83 - protein, 9 aa, Severe acute respiratory syndrome coronavirus 2
HLRIAGHHL

SEQ ID NO: 84 - protein, 9 aa, Severe acute respiratory syndrome-related coronavirus
ALNTPKDHI

SEQ ID NO: 85 - protein, 9 aa, Severe acute respiratory syndrome coronavirus 2
ALNTPKDHI

SEQ ID NO: 86 - protein, 9 aa, Severe acute respiratory syndrome-related coronavirus
LQLPQGTTL

SEQ ID NO: 87 - protein, 9 aa, Severe acute respiratory syndrome coronavirus 2
LQLPQGTTL

SEQ ID NO: 88 - protein, 10 aa, Severe acute respiratory syndrome-related coronavirus
GETALALLLL

SEQ ID NO: 89 - protein, 10 aa, Severe acute respiratory syndrome coronavirus 2
GDAALALLLL

SEQ ID NO: 90 - protein, 9 aa, Severe acute respiratory syndrome-related coronavirus
LALLLLDRL

SEQ ID NO: 91 - protein, 9 aa, Severe acute respiratory syndrome coronavirus 2
LALLLLDRL

SEQ ID NO: 92 - protein, 9 aa, Severe acute respiratory syndrome-related coronavirus
LLLDRLNQL

SEQ ID NO: 93 - protein, 9 aa, Severe acute respiratory syndrome coronavirus 2
LLLDRLNQL

SEQ ID NO: 94 - protein, 9 aa, Severe acute respiratory syndrome-related coronavirus
RLNQLESKV

SEQ ID NO: 95 - protein, 9 aa, Severe acute respiratory syndrome coronavirus 2
RLNQLESKM

SEQ ID NO: 96 - protein, 10 aa, Severe acute respiratory syndrome-related coronavirus
TKQYNVTQAF

SEQ ID NO: 97 - protein, 10 aa, Severe acute respiratory syndrome coronavirus 2
TKAYNVTQAF

SEQ ID NO: 98 - protein, 9 aa, Severe acute respiratory syndrome-related coronavirus
GMSRIGMEV

SEQ ID NO: 99 - protein, 9 aa, Severe acute respiratory syndrome coronavirus 2
GMSRIGMEV

SEQ ID NO: 100 - protein, 10 aa, Severe acute respiratory syndrome-related coronavirus
MEVTPSGTWL

SEQ ID NO: 101 - protein, 10 aa, Severe acute respiratory syndrome coronavirus 2
MEVTPSGTWL

SEQ ID NO: 102 - protein, 9 aa, Severe acute respiratory syndrome-related coronavirus
QFKDNVILL

SEQ ID NO: 103 - protein, 9 aa, Severe acute respiratory syndrome coronavirus 2
NFKDQVILL

SEQ ID NO: 104 - protein, 9 aa, Severe acute respiratory syndrome-related coronavirus
CLDAGINYV

SEQ ID NO: 105 - protein, 9 aa, Severe acute respiratory syndrome coronavirus 2
CLEASFNYL

SEQ ID NO: 106 - protein, 9 aa, Severe acute respiratory syndrome-related coronavirus
WLMWFIISI

SEQ ID NO: 107 - protein, 9 aa, Severe acute respiratory syndrome coronavirus 2
WLMWLIINL

SEQ ID NO: 108 - protein, 9 aa, Severe acute respiratory syndrome-related coronavirus
ILLLDQVLV

SEQ ID NO: 109 - protein, 9 aa, Severe acute respiratory syndrome coronavirus 2
ILLLDQALV

SEQ ID NO: 110 - protein, 9 aa, Severe acute respiratory syndrome-related coronavirus
LLCVLAALV

SEQ ID NO: 111 - protein, 9 aa, Severe acute respiratory syndrome coronavirus 2
SACVLAAEC

SEQ ID NO: 112 - protein, 9 aa, Severe acute respiratory syndrome-related coronavirus
ALSGVFCGV

SEQ ID NO: 113 - protein, 9 aa, Severe acute respiratory syndrome coronavirus 2
SLPGVFCGV

SEQ ID NO: 114 - protein, 9 aa, Severe acute respiratory syndrome-related coronavirus
TLMNVITLV

SEQ ID NO: 115 - protein, 9 aa, Severe acute respiratory syndrome coronavirus 2
TLMNVLTLV

SEQ ID NO: 116 - protein, 9 aa, Severe acute respiratory syndrome-related coronavirus
SMWALVISV

SEQ ID NO: 117 - protein, 9 aa, Severe acute respiratory syndrome coronavirus 2
SMWALIISV

(19) United States
(12) Patent Application Publication
(10) Pub. No.: US 2021/0277093 A1
(43) Pub. Date: Sep. 9, 2021

(54) IMMUNOTHERAPEUTIC COMPOSITIONS AND METHODS OF PRODUCTION FOR CORONAVIRUS

(71) Applicant: ADMA Biologics, Inc., Ramsey, NJ (US)

(72) Inventors: James Mond, Silver Spring, MD (US); Adam S. Grossman, Saddle River, NJ (US)

(21) Appl. No.: 17/196,846

(22) Filed: Mar. 9, 2021

Related U.S. Application Data
(60) Provisional application No. 62/987,213, filed on Mar. 9, 2020.
(60) Provisional application No. 62/994,624, filed on Mar. 25, 2020.

(51) Int. Cl. C07K 16/10; G01N 33/569; A61P 31/14; A61K 45/06; A61K 39/42

(52) U.S. Cl. CPC C07K 16/10; G01N 33/56983; A61P 31/14; A61K 2039/505; A61K 39/42; C07K 2317/10; G01N 2469/20; A61K 45/06

(57) ABSTRACT

Embodiments of the present disclosure relate generally to compositions and methods for the treatment and/or prevention of pathogenic viral infections, e.g., coronavirus infections. In particular, the present disclosure provides human plasma compositions and immunoglobulin prepared therefrom containing select antibody titers specific for coronavirus (e.g., SARS CoV-2), methods of identifying human donors and donor samples for use in the compositions, methods of manufacturing the compositions, and methods of utilizing the compositions for prophylactic administration and/or therapeutic treatment (e.g., passive immunization or immune-prophylaxis).

### CROSS REFERENCE TO RELATED APPLICATIONS

This application claims priority to and the benefit of U.S. Provisional Application No. 62/987,213, filed Mar. 9, 2020 and U.S. Provisional Application No. 62/994,624, filed Mar. 25, 2020, which are hereby incorporated by reference in their entireties.

### FIELD

The present disclosure relates to compositions and methods for the treatment and/or prevention of pathogenic infections (e.g., coronavirus infections). In particular, the present disclosure provides human plasma compositions and immunoglobulin prepared therefrom containing select antibody titers specific for SARS CoV-2, methods of identifying human plasma donors and donor samples for use in the compositions, and methods of utilizing the compositions for prophylactic administration and/or therapeutic treatment (e.g., passive immunization or immune-prophylaxis).

### BACKGROUND

Commercially available immunoglobulins are derived from pooled human serum, collected, processed, and distributed for sale by the blood and plasma products industry. The first purified human immunoglobulin G (IgG) preparation used clinically was immune serum globulin which was produced in the 1940s (Cohn, E. J., et al. J. Am Chem. Soc., 68:459-475 (1946) and Oncely, J. L. et al., J. Am Chem Soc. 71:541-550 (1949)). The immunoglobulin produced by this method demonstrated a molecular distribution having a high molecular weight, when analyzed by way of high resolution size exclusion chromatography. Immunoglobulin has historically been used primarily to prevent infections in patients who are immune deficient. Immunoglobulin obtained from the plasma of thousands of different donors contains antibodies to many of the pathogens that the donor individuals have encountered in their lifetime and it is these antibodies when infused into patients that prevent them from suffering serious infections.

However, significant limitations exist with currently available immunoglobulin products. Since immunoglobulin from thousands of random donors are pooled, the antibody titers to the many infectious organisms (e.g., viruses) for which protection is sought varies greatly and very often are not sufficient to meet the immune needs of individuals (e.g., in case of a serious infection with a pathogen).

In addition, pools of immune globulin contain only antibodies to pathogens to which the person was exposed and not to pathogens to which the individual had no immunological exposure. Thus, pathogens that undergo extensive mutations or pathogens that might be carried by non-human vectors and develop mechanisms to infect humans will be unable to stimulate an immediate anamnestic immunological response that would be required to prevent infection.

### SUMMARY

The present disclosure relates to compositions and methods for the treatment and/or prevention of pathogenic infections such as, for example, coronavirus infections for which standard immune globulin pools will not adequately provide. In particular, the present disclosure provides pooled human plasma compositions and immunoglobulin prepared therefrom, methods of identifying human plasma for use in the compositions, and methods of utilizing the compositions for prophylactic administration and/or therapeutic treatment (e.g., passive immunization or immune-prophylaxis).

In accordance with the embodiments provided herein, the present disclosure provides pooled plasma compositions and/or immunoglobulin prepared therefrom having increased neutralizing antibody titers against specific viral pathogens, such as for example, coronavirus (coronavirus OC43, coronavirus 229E, coronavirus NL63, coronavirus HKU1, MERS-CoV, SARS-CoV, SARS-CoV-2 (COVID-19)). As described further herein, the compositions include pooled plasma samples and/or immunoglobulin prepared therefrom, which are obtained from a plurality of selected donor human subjects (e.g., 50, 100, 200, 300, 400, 500 or more subjects). In some embodiments, a pooled sample comprising higher neutralizing antibody titers against one virus also has proportionally higher neutralizing antibody titers against other viruses. For example, pooled plasma samples can be obtained from a plurality of selected human donor subjects having increased antibody titers against a coronavirus (e.g., at least 1.2 fold greater than antibody titers from a corresponding control sample and/or an antibody neutralization titer of at least 40 to about 30,000). Additionally, in some embodiments, pooled plasma samples can be obtained from a plurality of selected human donor subjects having increased antibody titers against a respiratory pathogent (e.g., RSV (e.g., at least 1.2 fold greater than antibody titers from a corresponding control sample or an antibody neutralization titer of at least 1000 to 8000)), and these pooled plasma samples can also have proportionally increased antibody titers against a coronavirus (e.g., at least 1.2 fold greater than antibody titers from a corresponding control sample or an antibody neutralization titer of at least 40 to about 30,000).

In one embodiment, the present disclosure provides a composition comprising pooled plasma samples obtained from a plurality of selected human subjects (e.g., 50, 100, 200, 300, 400, 500 or more human plasma donors), wherein the pooled plasma comprises elevated levels (e.g., selected, consistent and/or standardized levels), compared to the pathogen-specific antibody titers found in a mixture of plasma samples obtained from a plurality random human subjects (e.g., 50, 100, 200, 300, 400, 500 or more human plasma donors), of pathogen-specific antibody titers to one or more (e.g., two, three, four, or more) coronaviruses (e.g., coronavirus OC43, coronavirus 229E, coronavirus NL63, coronavirus HKU1, MERS-CoV, SARS-CoV, and/or SARS-CoV-2 (COVID-19). Embodiments of the present disclosure are not limited by the type of viral pathogens for which the pooled plasma comprises elevated levels of specific antibody titers. For example, the pooled plasma composition may comprise elevated levels of pathogen-specific antibody titers to one or more of coronavirus OC43, coronavirus 229E, coronavirus NL63, coronavirus HKU1, MERS-CoV, SARS-CoV, and/or SARS-CoV-2 (COVID-19)). In another embodiment, the pooled plasma from selected plasma donors comprises elevated levels, compared to the pathogen-specific antibody titers found in a mixture of plasma samples obtained from a plurality of random human subjects, of pathogen-specific antibody titers to two or more non-coronavirus pathogens, for example, respiratory syncytial virus (RSV), influenza A virus, influenza B virus, parainfluenza virus type 1, parainfluenza virus type 2, metapneumovirus, or any other respiratory pathogen known in the art or described herein. In still another embodiment, the pooled plasma comprises elevated levels, compared to the pathogen-specific antibody titers found in a mixture of plasma samples obtained from a plurality of random human subjects, of pathogen-specific antibody titers to three, four, five, six or more viral pathogens described herein. In one embodiment, the pooled plasma comprises a coronavirus-specific antibody titer that is at least 1.2 fold greater (e.g., 1.2 fold, 1.3 fold, 1.4 fold, 1.5 fold, 1.6 fold, 1.7 fold, 1.8 fold, 1.9 fold, 2 fold, 3 fold, 4 fold, 5 fold 6 fold, 7 fold, 8 fold, 9 fold, 10 fold or more) than the corresponding antibody titer found in a mixture of plasma samples obtained from 100 or more random human subjects. In some embodiments, the antibody neutralization titer for a coronavirus is at least 40 to about 30,000. In another embodiment, the pooled plasma comprises pathogen-specific antibody titers to at least a second viral pathogen selected from respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, and SARS-CoV-2 (COVID-19), that is significantly elevated (e.g., at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0, 7.0, 8.0 or more fold) compared to the pathogen-specific antibody titers found in a mixture of plasma samples obtained from a plurality of random human subjects.

Thus, in one embodiment, the present disclosure provides means for the identification and characterization of plasma and/or immune globulin compositions containing a desired functional, neutralizing coronavirus antibody titer rather than one in which only the total amount of IgG is known.

In accordance with these embodiments, anti-SARS CoV-2 antibody titer present in a donor plasma sample can be identified by total antibody binding (e.g., using an ELISA). For example, in some embodiments, the present disclosure provides a pooled plasma composition comprising plasma from a plurality of plasma donors wherein each donor's plasma exhibits an SARS CoV-2 antibody titer that is at least 1.2 fold greater (e.g., 1.2, 1.5, 2, 2.5, 3, 3.5, 4.5, 5, 6, 7, 8, 9, 10 fold or greater, or any value therebetween) than the SARS CoV-2-specific antibody titer found in a negative control (e.g., plasma devoid of coronavirus antibodies, or a mixture of plasma samples obtained from a plurality of random, non-convalescent human subjects). In some embodiments, plasma samples are selected based upon the total amount of SARS CoV-2-specific antibody titer (e.g., only those plasma samples that display a threshold (e.g., 2 fold or higher) SARS CoV-2-specific antibody titer are selected). In some embodiments, the selected plasma samples are assayed to characterize SARS CoV-2 neutralizing antibody titer in the samples. In further embodiments, plasma samples are selected based upon the SARS CoV-2 neutralizing antibody titer (e.g., only those plasma samples that display a SARS CoV-2 neutralizing antibody titer in the top 50%, 55%, 60%, 65%, 70%, 75%, 80%, 85%, 90% or higher of all plasma samples tested) are selected. In some embodiments, plasma samples are selected based upon both the SARS CoV-2-specific antibody titer and the SARS CoV-2-specific neutralizing titer. For example, as disclosed herein, it was determined that of all convalescent, COVID-19 convalescent plasma analyzed, there exist plasma samples that display a high level of a SARS CoV-2-antibody binding, but lack a corresponding high SARS CoV-2-neutrlizing titer. Compositions and methods disclosed herein are useful at identifying these plasma samples in order to specifically exclude these plasma samples from use in a pooled plasma composition or immune globulin prepared therefrom disclosed herein. The disclosure is not limited to any particular assay for determining neutralizing antibody titer. Indeed, any assay available in the art may be utilized. In some embodiments, a plaque/focus reduction neutralization test (P/FRNT) is performed. In further embodiments, an automated high-throughput antibody neutralization assay based on foci and plaque reduction is used. In other embodiments, a virus reduction neutralization test (VRNT) is utilized (see, e.g., Whiteman et al., Am J Trop Med Hyg. 2018 December; 99(6):1430-1439). In still other embodiments, a pseudovirus neutralization assay is utilized (Creative Diagnostics, Shirley, N.Y.). In some embodiments, a multiplexed bead-based SARS-CoV-2 serological assay is used (NIST, Gaithersburg, Md.).

The disclosure provides a method of producing an immune globulin comprising obtaining a plurality of plasma samples from a plurality of plasma donors (e.g., COVID-19 convalescent plasma donors or COVID-19 vaccinated donors), conducting a first assay on each plasma sample to measure total anti-SARS CoV-2 antibody titer, selecting, based upon the first assay, plasma samples having a total anti-SARS CoV-2 antibody binding titer that is about two-fold or higher (e.g., 3-, 4-, 5-, 6-, 7-, 8-, 9-, 10-fold or higher) than the amount of total anti-SARS CoV-2 antibody binding titer in a control sample, conducting a second assay on each selected plasma sample from step (3) to measure SARS CoV-2 neutralizing antibody titer, identifying, based upon the second assay, plasma samples having a neutralizing antibody titer in the lower 65% of all plasma samples assayed and excluding the identified plasma samples from further processing, pooling the non-excluded plasma samples, and preparing immune globulin from the pooled plasma samples. In some embodiments, each of the plurality of plasma donors is a COVID-19 convalescent plasma donor. In other embodiments, each of the plurality of plasma donors is a COVID-19 vaccinated plasma donor. In some embodiments, the control sample is a mixture of plasma samples obtained from random human plasma donors (e.g., 50, 100, 150, 200, 250, 500, 1000 or more plasma donors or number therebetween). In other embodiments, the control sample is a commercially available immune globulin. In some embodiments, plasma samples having a neutralizing antibody titer in the lower 70% of all plasma samples assayed are identified and excluded from further processing. In still other embodiments, plasma samples having a neutralizing antibody titer in the lower 75% of all plasma samples assayed are identified and excluded from further processing. In other embodiments, plasma samples having a neutralizing antibody titer in the lower 80% of all plasma samples assayed are identified and excluded from further processing. The disclosure is not limited by the number of non-excluded, pooled plasma samples. In some embodiments, the number of non-excluded, pooled plasma samples is 250-500 or more. In other embodiments, the number of non-excluded, pooled plasma samples is 500-1000 or more. In some embodiments, the immune globulin is prepared using a cold alcohol fractionation process that isolates the immune globulin fraction from the pooled plasma as a solution. In further embodiments, the immune globulin is combined with a pharmaceutically acceptable carrier.

In some embodiments, the disclosure provides a method of producing an immune globulin comprising obtaining a plurality of plasma samples from human plasma donors vaccinated with one or more coronavirus vaccines (e.g., one or more vaccines disclosed herein or available in the art); conducting a first assay on each plasma sample to measure total anti-SARS CoV-2 antibody titer, selecting, based upon the first assay, plasma samples having a total anti-SARS CoV-2 antibody binding titer that is about two-fold or higher (e.g., 3-, 4-, 5-, 6-, 7-, 8-, 9-, 10-fold or higher) than the amount of total anti-SARS CoV-2 antibody binding titer in a control sample, conducting a second assay on each selected plasma sample from step (3) to measure SARS CoV-2 neutralizing antibody titer, identifying, based upon the second assay, plasma samples having a neutralizing antibody titer in the lower 65% of all plasma samples assayed and excluding the identified plasma samples from further processing, pooling the non-excluded plasma samples, and preparing immune globulin from the pooled plasma samples. In some embodiments, immunoglobulin is prepared using a cold alcohol fractionation process (e.g., that isolates the immune globulin fraction from the pooled plasma as a solution). In some embodiments, the disclosure provides a pharmaceutical composition comprising an immune globulin obtained by the methods disclosed. In further embodiments, the disclosure provides a method of treating a human patient comprising administering an immune globulin obtained by the disclosed methods to a subject/patient. In some embodiments, the immune globulin reduces viral load in the lung and/or nose of a subject administered the composition compared to a control subject not receiving the composition. In some embodiments, the immune globulin reduces lung histopathology of a subject administered the composition compared to a control subject not receiving the composition. In one embodiment, the pooled plasma comprises plasma samples obtained from 50-3000 or more (e.g., more than 50, 100, 200, 300, 400, 500, 750, 1000, 1250, 1500, 1750, 2000, 2500, 3000, 3500, 4000 or more) human subjects (e.g., COVID-19 convalescent patients, or, vaccinated human plasma donors). In one preferred embodiment, the pooled plasma comprises plasma samples obtained from 100-1000 human subjects. In another preferred embodiment, the pooled plasma comprises plasma samples obtained from at least 1000 human subjects. In one embodiment, the composition comprising pooled plasma samples further comprises a pharmaceutically acceptable carrier (e.g., natural and/or non-naturally occurring carriers). In one embodiment, the pooled plasma composition is utilized to prepare immunoglobulin (e.g., for intravenous administration to a subject). In one embodiment, the pooled plasma composition and/or immunoglobulin provides a therapeutic benefit to a subject administered the composition that is not achievable via administration of a mixture of plasma samples obtained from a plurality of random human subjects and/or immunoglobulin prepared from same. Embodiments of the present disclosure are not limited by the type of therapeutic benefit provided. Indeed, a variety of therapeutic benefits may be attained including those described herein. In one embodiment, the pooled plasma and/or immunoglobulin possesses enhanced viral neutralization properties compared to a mixture of plasma samples obtained from a plurality of random human subjects or immunoglobulin prepared from same. In a further embodiment, the enhanced viral neutralization properties reduce and/or prevent infection in a subject administered the composition for a duration of time that is longer than, and not achievable in, a subject administered a mixture of plasma samples obtained from a plurality of random human subjects. For example, in one embodiment, immunoglobulin prepared from pooled plasma according to the present disclosure (e.g., characterized, selected and blended according to the embodiments of the present disclosure) that is administered to a subject results in a significant, concentration dependent anti-coronavirus neutralization activity, specific neutralization activity that is not achieved or achievable using immunoglobulin prepared from randomly pooled plasma samples (e.g., over a period of hours, days, weeks or longer). While an understanding of a mechanism is not needed to practice aspects of the disclosure, and while the present disclosure is not limited to any particular mechanism, in some embodiments, identification and selection of plasma donors that display both a threshold level of SARS CoV-2 antibody binding and a threshold level of SARS CoV-2 neutralization activity, according to processes and methods disclosed, provides pooled plasma composition and/or immunoglobulin prepared therefrom that possess therapeutic activity and properties superior to and/or absent from conventional convalescent plasma. For example, in some embodiments, plasma samples from convalescent plasma donors (e.g., donors that have recovered from COVID-19) are characterized for SARS CoV-2 antibody binding and SARS CoV-2 neutralization activity, and those samples that display a moderate to high levels of SARS CoV-2 antibody binding but that do not display SARS CoV-2 neutralizing activity (e.g., neutralizing activity that correlates with the total amount of antibody binding) are specifically excluded from use in a pooled plasma composition and/or immunoglobulin prepared therefrom. While an understanding of a mechanism is not needed to practice aspects of the disclosure, and while the present disclosure is not limited to any particular mechanism, in some embodiments, excluding plasma containing moderate to high levels of SARS CoV-2 binding antibodies that lack neutralizing activity from a pooled plasma composition provides a significantly more effective pooled plasma composition and/or immunoglobulin prepared therefrom.

In one embodiment, the therapeutic benefit of a pooled plasma and/or immunoglobulin of the present disclosure is enhanced viral neutralization properties that reduce or prevent infection (e.g., coronavirus infection) in a subject administered the pooled plasma and/or immunoglobulin for a duration of time that is longer than, and not achievable in, a subject administered a mixture of pooled plasma and/or immunoglobulin prepared from same obtained from a plurality of random human subjects. In one embodiment, the therapeutic benefit is a significant reduction in viral load of a subject administered the pooled plasma and/or immunoglobulin compared to a control subject not receiving same. In a further embodiment, the pooled plasma and/or immunoglobulin significantly reduces lung histopathology in a subject administered the pooled plasma and/or immunoglobulin compared to a control subject not receiving same. In yet a further embodiment, the pooled plasma and/or immunoglobulin significantly reduces the level of pathogenic viral RNA in a tissue selected from lung, liver and kidney in a subject administered the pooled plasma and/or immunoglobulin compared to a control subject. In one embodiment, a subject administered immunoglobulin prepared from pooled plasma according to the present disclosure displays a mean fold increase in coronavirus neutralization titer that is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0, 7.0, 8.0 fold or more, or any ranger therebetween, at a time point of at least 1-14 days (e.g., 14 day, 15 days, 16 days, 17 days, 18 days, 19 days or more) post administration of the immunoglobulin. Embodiments of the present disclosure are not limited by the amount of immunoglobulin administered to a subject. In one embodiment, a subject is administered between 250-2500 mg/kg of the immunoglobulin one time, or daily for two or more days (e.g., 2, 3, 4, or more consecutive days). In one embodiment, a subject is administered 1500 mg/kg of immunoglobulin on day one and 750 mg/kg immunoglobulin on day 2. In another embodiment, a subject is administered 750 mg/kg of immunoglobulin on day one and 750 mg/kg immunoglobulin on day 2. In one embodiment, the pooled plasma and/or immunoglobulin prepared from same reduces the incidence of infection in a subject administered the composition. In another embodiment, a pooled plasma and/or immunoglobulin prepared from same reduces the number of days a subject administered the pooled plasma and/or immunoglobulin is required to be administered antibiotics (e.g., to treat infection). In yet another embodiment, a pooled plasma and/or immunoglobulin prepared from the same increases the trough level of circulating anti-SARS CoV-2-specific, functional antibodies in a subject (e.g., increases the level of neutralizing titers specific for SARS CoV-2, thereby providing protective levels of anti-SARS CoV-2-specific antibodies between scheduled dates of administration of the pooled plasma and/or immunoglobulin prepared from same that are not maintained in a subject administered a mixture of plasma samples obtained from a plurality of more random human subjects (e.g., 50, 100, 200, 300, 400, 500 or more subjects) or immunoglobulin prepared from same).

In another embodiment, the present disclosure provides an immunotherapeutic composition comprising pooled plasma samples obtained from a plurality of selected human subjects, wherein the pooled plasma comprises elevated levels, compared to the pathogen-specific antibody titers found in a mixture of plasma samples obtained from a plurality of random human subjects, of pathogen-specific antibody titers to two or more viral pathogens selected from respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, and SARS-CoV-2 (COVID-19); and a pharmaceutically acceptable carrier. In one embodiment, an immunotherapeutic composition provided herein further comprises one or more biologically active agents. Embodiments of the present disclosure are not limited to the type of biologically active agent/material. Indeed, a variety of biologically active agents/materials may be used including, but not limited to, antibodies, anti-toxin material, anti-inflammatory agent, anti-cancer agent, antimicrobial agent, therapeutic agent, antihistamine, cytokine, chemokine, vitamin, mineral, or the like.

In one embodiment, the biologically active agent is an anti-toxin agent. In one embodiment, the anti-toxin agent is a mono-specific, bi-specific or multi-specific antibody with specificity toward a viral, bacterial or fungal toxin. In a further embodiment, the bacterial or fungal toxin is selected from Botulinum neurotoxin, Tetanus toxin, E. coli toxin, Clostridium difficile toxin, Vibrio RTX toxin, Staphylococcal toxins, Cyanobacteria toxin, and mycotoxins. In another embodiment, the immunotherapeutic composition further comprises an aliquot of a single or multiple monoclonal antibodies with a single or multiple specificities (e.g., the immunogenic composition may be spiked with one or more antibodies or biologically active material (e.g., a monoclonal antibody of any specificity, an anti-toxin agent, etc.)). Embodiments of the present disclosure are not limited by the type of one or more antibodies that are added to (e.g., spiked into) the immunogenic composition. Indeed, any one or more antibodies (e.g., specific for a pathogen or pathogen product) may be used including, but not limited to standard antibodies, bi-specific antibodies, multi-specific antibodies, or the like known in the art (e.g., specific for one or a multiplicity of antigens).

In some embodiments, compositions of the present disclosure (e.g., pooled plasma samples and/or immunoglobulin prepared therefrom) are spiked with one or more antibodies that bind to one or more epitope(s) of a target antigen (e.g., epitope of a viral pathogen). The presence of one or more antibodies in the compositions described herein can enhance the therapeutic effects of the compositions, including treating and/or preventing one or more aspects of the viral infection. In some embodiments, the one or more antibodies have been shown to bind a specific target antigen and may also have been shown to have therapeutic efficacy against a given pathogen, such as a virus. In some embodiments, existing antibodies added to the therapeutic compositions of the present disclosure enhance therapeutic efficacy, and include, but are not limited to, antibodies that bind one or more antigenic regions of a virus that are conserved among viruses or viral subtypes, that are unique among viruses or viral subtypes (e.g., variants), and/or are present in a particular virus because of genetic recombination.

As would be recognized by one of ordinary skill in the art based on the present disclosure, antibodies that bind one or more epitopes of a viral pathogen can be generated and added to the compositions of the present disclosure (e.g., pooled plasma samples and/or immunoglobulin prepared from same). In some embodiments, antibodies are generated against one or more epitopes of a coronavirus antigen (e.g., coronavirus OC43, coronavirus 229E, coronavirus NL63, coronavirus HKU1, MERS-CoV, SARS-CoV, or SARS-CoV-2 (COVID-19)). In accordance with these embodiments, the present disclosure includes any method for generating a coronavirus antibody that binds at least one epitope of a coronavirus antigen. Such antibodies can be generated using amino acid sequence information currently available corresponding to any of the known coronavirus strains, as well as that of any future coronavirus strains identified, by methods known in the art, non-limiting examples of which are described further below. In some embodiments, antibodies are generated that bind an epitope or epitopes present in more than one coronavirus strain (e.g., cross-reactive antibodies that recognize a conserved region of a coronavirus protein). In some embodiments, antibodies are generated that bind an epitope or epitopes present in a single coronavirus strain (e.g., antibodies that recognize a unique region of a coronavirus protein). In accordance with these embodiments, the sequence of SARS-CoV-2 can be accessed via NCBI GenBank accession code MN908947; the sequence of SARS-CoV can be accessed via NCBI GenBank accession code AY274119; the sequence of MERS-CoV can be accessed via NCBI GenBank accession code NC_019843; the sequence of HKU1 (beta coronavirus) can be accessed via NCBI GenBank accession code KF686346; the sequence of OC43 (beta coronavirus) can be accessed via NCBI GenBank accession code NC_006213; the sequence of NL63 (alpha coronavirus) can be accessed via NCBI GenBank accession code NC_005831; and the sequence of 229E (alpha coronavirus) can be accessed via NCBI GenBank accession code NC_002645.

In some embodiments, one or more coronavirus antigens (e.g., comprising one or more antigenic epitopes of a coronavirus antigen described herein) are used as or in a vaccine to immunize a subject (e.g., to generate a coronavirus-specific immune response). In some embodiments, a subject administered the vaccine is used as a plasma donor (e.g., to generate an immune globulin composition described herein). In some embodiments, the present disclosure includes human plasma immunoglobulin compositions containing antibodies specific for a coronavirus or coronaviruses obtained from human donor samples that have been immunized with a coronavirus vaccine and methods of utilizing the compositions for prophylactic administration and/or therapeutic treatment (e.g., passive immunization or immune-prophylaxis). In some embodiments, immunoglobulin compositions of the present disclosure can be obtained from pooled plasma samples obtained from a plurality of donor human subjects (e.g., 50, 100, 200, 300, 400, 500 or more subjects) that have been immunized with one or more coronavirus antigens (e.g., comprising one or more antigenic epitopes of a coronavirus antigen described herein) derived from a coronavirus (e.g., coronavirus OC43, coronavirus 229E, coronavirus NL63, coronavirus HKU1, MERS-CoV, SARS-CoV, or SARS-CoV-2 (COVID-19)).

In some embodiments, one or more epitopes of a coronavirus antigen find use in an antibody binding assay (e.g., ELISA) and/or a neutralization assay described herein.

In another embodiment, the present disclosure provides a method of providing immunotherapy to a subject (e.g., a subject in need thereof (e.g., a subject with disease or at risk for disease)) comprising administering to the subject a therapeutically effective amount of a composition comprising pooled plasma samples or immunoglobulin prepared therefrom obtained from a plurality of selected human subjects (e.g., 50, 100, 200, 300, 400, 500 or more subjects), wherein the pooled plasma comprises elevated levels, compared to the pathogen-specific antibody titers found in a mixture of plasma samples obtained from a plurality of random human subjects (e.g., 50, 100, 200, 300, 400, 500 or more subjects), of pathogen-specific antibody titers to one or more (e.g., two, three, four, five or more) viral pathogens selected from coronavirus (e.g., coronavirus OC43, coronavirus 229E, coronavirus NL63, coronavirus HKU1, MERS-CoV, SARS-CoV, SARS-CoV-2 (COVID-19)), respiratory syncytial virus (RSV), influenza A virus, influenza B virus, parainfluenza virus type 1, parainfluenza virus type 2, and metapneumovirus. In one embodiment, the immunotherapy is used to prophylactically treat infection associated with a microbial pathogen. In another embodiment, the immunotherapy is used to therapeutically treat infection associated with a microbial pathogen.

An advantage of the compositions and methods described herein is that many embodiments do not require the subject to be given additional drugs to treat their risk of infection, and therefore they are spared adverse side effects or interactions with other therapies. Another advantage of the compositions and methods described herein is that the compositions and methods may be used to treat or prevent disease wherein drugs or other conventional treatments do not exist to treat or prevent the disease (e.g., compositions and methods of the present disclosure can be used to treat and/or prevent infection with coronavirus). Another advantage of the compositions and methods described herein is that immunoglobulin provided by the present disclosure provides protection and therapeutic benefit to a coronavirus infected patient. In one embodiment, compositions and methods of the present disclosure are utilized for prophylactic and/or therapeutic treatment of infection for which there exists no known cure (e.g., various viral illnesses). In some embodiments, compositions and methods of the present disclosure are utilized for prophylactic and/or therapeutic treatment of infections. In some embodiments, compositions and methods of the present disclosure are utilized for prophylactic and/or therapeutic treatment of a subject that harbors a non-competent immune system (e.g., in which treatment with antibiotic or other conventional antimicrobial therapy would have little to no value).

In other embodiments, compositions and methods of the present disclosure are utilized to reduce the risk of a subject developing an infection (e.g., a respiratory infection). Embodiments of the present disclosure are not limited by the type of subject treated with the compositions and methods provided herein. Indeed, a variety of subjects may be so treated, including, but not limited to, a subject at risk of developing an infection (e.g., respiratory or other type of infection, thereby reducing the risk of developing infection in a subject having an elevated risk of infection). In one embodiment, the immunotherapy provides the subject with prophylactic immunity against one or more pathogens selected from coronavirus (e.g., coronavirus OC43, coronavirus 229E, coronavirus NL63, coronavirus HKU1, MERS-CoV, SARS-CoV, SARS-CoV-2 (COVID-19)), and respiratory syncytial virus (RSV). However, embodiments of the present disclosure are not so limited. Indeed, immunotherapy with the compositions and methods of the present disclosure may provide the subject with prophylactic immunity to any of the microbial pathogens described herein.

In another embodiment, the present disclosure provides a method of producing a pooled plasma composition, comprising obtaining plasma samples from human subjects; characterizing the pathogen-specific antibody titer, within a subset of the plasma samples, for one or more viral pathogens selected from respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, SARS-CoV-2 (COVID-19); selecting, based upon the antibody titers characterized, plasma samples that have elevated levels, compared to a control value (e.g., the pathogen-specific antibody titers found in a mixture of plasma samples obtained from a plurality of random human subjects), of pathogen-specific antibody titers to one or more pathogens selected from coronavirus (e.g., coronavirus OC43, coronavirus 229E, coronavirus NL63, coronavirus HKU1, MERS-CoV, SARS-CoV, SARS-CoV-2 (COVID-19)), respiratory syncytial virus (RSV), influenza A virus, influenza B virus, parainfluenza virus type 1, parainfluenza virus type 2, metapneumovirus; pooling the selected plasma samples with other plasma samples to generate the pooled plasma composition, wherein the pooled plasma composition comprises pathogen-specific antibody titers to one or more respiratory pathogens selected from respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, SARS-CoV-2 (COVID-19), the one or more titers being elevated at least 1.1 fold compared to a control value (e.g., the pathogen-specific antibody titers in a mixture of plasma samples obtained from a plurality of random human subjects). In one embodiment, the method comprises selecting, based upon the antibody titers characterized, plasma samples that have elevated levels, compared to the pathogen-specific antibody titers found in a mixture of plasma samples obtained from a plurality of random human subjects, of pathogen-specific antibody titers to two or more viral pathogens selected from respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, and SARS-CoV-2 (COVID-19).

In a further embodiment, the method comprises selecting, based upon the antibody titers characterized, plasma samples that have elevated levels, compared to the pathogen-specific antibody titers found in a mixture of plasma samples obtained from a plurality of random human subjects, of pathogen-specific antibody titers to two, three, four or more pathogens selected from coronavirus (e.g., coronavirus OC43, coronavirus 229E, coronavirus NL63, coronavirus HKU1, MERS-CoV, SARS-CoV, SARS-CoV-2 (COVID-19)), respiratory syncytial virus (RSV), influenza A virus, influenza B virus, parainfluenza virus type 1, parainfluenza virus type 2, and metapneumovirus. In one embodiment, the pooled plasma composition comprises pathogen-specific antibody titers to at least two or more respiratory pathogens selected from coronavirus (e.g., coronavirus OC43, coronavirus 229E, coronavirus NL63, coronavirus HKU1, MERS-CoV, SARS-CoV, SARS-CoV-2 (COVID-19)), respiratory syncytial virus (RSV), influenza A virus, influenza B virus, parainfluenza virus type 1, parainfluenza virus type 2, and metapneumovirus that are each elevated at least 1.1 fold compared to the pathogen-specific antibody titers found in a mixture of plasma samples obtained from a plurality of random human subjects. In one embodiment, the pooled plasma composition comprises pathogen-specific antibody titers to at least two or more respiratory pathogens selected from coronavirus (e.g., coronavirus OC43, coronavirus 229E, coronavirus NL63, coronavirus HKU1, MERS-CoV, SARS-CoV, SARS-CoV-2 (COVID-19)), respiratory syncytial virus (RSV), influenza A virus, influenza B virus, parainfluenza virus type 1, parainfluenza virus type 2, and metapneumovirus that are each elevated at least 1.2 fold compared to the pathogen-specific antibody titers found in a mixture of plasma samples obtained from a plurality of random human subjects. In another embodiment, the pooled plasma composition comprises a coronavirus-specific antibody titer that is at least 1.2 fold greater (e.g., 1.2, 1.5, 2, 2.5, 3, 3.5, 4.5, 5, 6, 7, 8, 9, 10 fold or more or any value therebetween) than the coronavirus-specific antibody titer found in a mixture of plasma samples obtained from a plurality of random human subjects. For example, in one embodiment, the present disclosure provides a method of producing a pooled plasma composition (e.g., containing a specific, elevated antibody titer for coronavirus and a specific, elevated antibody titer for at least a second viral pathogen), from at least 100 human plasma donors, comprising obtaining plasma samples from selected human plasma donors, wherein the selected human donors are identified via characterizing the specific titer of antibodies to a coronavirus in a plasma sample from a human donor, wherein characterizing the specific titer of antibodies comprises a first, plasma screening assay utilized to determine total SARS CoV-2 antibody binding titer, a second assay of plasma samples selected from the first assay, wherein the second assay determines SARS CoV-2 neutralizing titer, a second selection of plasma samples displaying a desired neutralization titer and exclusion of plasma samples lacking the desired neutralization titer, wherein only those samples that display a desired, threshold total SARS CoV-2 antibody binding titer (e.g., 2 fold or higher compared to a control) and a threshold SARS CoV-2 neutralizing titer (e.g., the top 25%, top 20%, top 15%, top 10%, top 5% or greater, or any value therebetween) are pooled together to generate a pooled plasma composition (e.g., from which immunoglobulin is produced).

In some embodiments, the present disclosure provides a composition comprising pooled plasma samples (e.g., a therapeutic composition) comprising plasma from a plurality of donors (e.g., 100 or more human donors), that have been clinically diagnosed with an infection by a viral pathogen, such as an infection from one or more of a coronavirus (e.g., coronavirus OC43, coronavirus 229E, coronavirus NL63, coronavirus HKU1, MERS-CoV, SARS-CoV, SARS-CoV-2 (COVID-19)), respiratory syncytial virus (RSV), influenza A virus, influenza B virus, parainfluenza virus type 1, parainfluenza virus type 2, and metapneumovirus. In some embodiments, plurality of donors have recovered or are recovering from the viral infection. In some embodiments, a clinical diagnosis of a viral infection is carried out by a medical or laboratory professional and involves obtaining a sample(s) from the plurality of donors (e.g., blood sample, plasma sample, serum sample, fecal sample, urine sample cheek swab, sputum sample, and the like), and testing the sample using any of a variety of antibody-based and/or molecular (e.g., PCR) and/or clinical chemistry testing protocols to identify the presence of the virus and/or one or more physiological responses from the subject that correlates to the presence/absence of the virus. A clinical diagnosis generally involves a physiological readout based on the sample that indicates whether a subject has recovered or is recovering from the infection. A physiological indication of recovery can include, but is not limited to, presence/absence of an antibody, a nucleic acid, a metabolite, and the like. In some embodiments, a clinical diagnosis can indicate whether a subject has a coronavirus infection (e.g., coronavirus OC43, coronavirus 229E, coronavirus NL63, coronavirus HKU1, MERS-CoV, SARS-CoV, SARS-CoV-2 (COVID-19)), as well as whether the subject has recovered or is recovering from the coronavirus infection. In some embodiments, the one or more of the plurality of human plasma donors have been clinically diagnosed with infection by the coronavirus and have recovered from the infection. In some embodiments, the one or more of the plurality of human plasma donors have been clinically diagnosed with an infection from the at least a second virus and have recovered from the infection. In some embodiments, the one or more of the plurality of human plasma donors have not been clinically diagnosed with infection by the coronavirus. In some embodiments, the one or more of the plurality of human plasma donors have not been clinically diagnosed with an infection from the at least a second virus. In some embodiments, the one or more of the plurality of human donors have been selected based on at least one pre-preselection criterion, including but not limited to occupation (e.g., teacher, flight attendant, healthcare professional), proximity to an infection hotspot, degree of contact to other humans, and the like.

In some embodiments, the present disclosure provides a composition comprising pooled plasma samples (e.g., a therapeutic composition) comprising plasma from a plurality of donors (e.g., 100 or more human donors), wherein all or a subset of the plurality of donors possess a high titer of pathogen-specific antibodies to one or more viral pathogens as a result of administration of an immunogenic composition (e.g., viral vaccine) comprising antigens to the plurality of pathogens, which generates an immunogenic response in the subject. In some embodiments, the plurality of human plasma donors have been clinically diagnosed as having or not having a particular viral infection. In some embodiments, the plurality of human plasma donors have not been clinically diagnosed as having or not having a particular viral infection. As described further herein, compositions of the present disclosure include pooled plasma samples from this plurality of donors that have received a coronavirus vaccine (e.g., vaccine for one or more of coronavirus OC43, coronavirus 229E, coronavirus NL63, coronavirus HKU1, MERS-CoV, SARS-CoV, SARS-CoV-2 (COVID-19)).

In one embodiment, compositions are provided that comprise a plurality of different types of antibodies (e.g., directed to different pathogens (e.g., viral pathogens, bacterial pathogens, eukaryotic pathogens, etc.), recognize different antigens, recognize different epitopes, etc.) and are enriched (e.g., elevated titer) for at least two different antibodies or sets of antibodies (e.g., directed to different pathogens, recognize different antigens, recognize different epitopes, etc.). In particular embodiments, compositions comprise tailored antibody pools. In some embodiments, at least from about 0.01% to about 70% of the total immunoglobulin present in the composition is directed to one or more targeted pathogens, although embodiments of the present disclosure are not so limited (e.g., the composition may comprise less than 0.01% or more than 70% of immunoglobulin directed to targeted pathogens). Immunoglobulin directed to targeted pathogens may comprise >0.1% >2%, >5%, >10%, >20%, >30%, >40%, >50%, >60%, >70%, >80%, or >90% of the total immunoglobulin present in the composition. In certain embodiments, a composition comprises two or more immunoglobulins to targeted pathogens, each present at greater than 1% of total immunoglobulin present in the composition (e.g., two or more immunoglobulins to targeted pathogens present at greater than 1.5%, 2.0%, 3.0%, 4.0%, 5.0% or more of total immunoglobulin, two or more immunoglobulins to targeted pathogens present at greater than 10% of total immunoglobulin, two or more immunoglobulins to targeted pathogens present at greater than 15% of total immunoglobulin, two or more immunoglobulins to targeted pathogens present at greater than 20% of total immunoglobulin, two or more immunoglobulins to targeted pathogens present at greater than 25% of total immunoglobulin, etc.).

Any suitable method for obtaining plasma, antibody samples, pooled plasma compositions and/or immunoglobulin from same are within the scope of the present disclosure. Further, any suitable method for producing, manufacturing, purifying, fractionating, enriching, etc. antibody samples and/or plasma pools is within the bounds of the present disclosure. Exemplary techniques and procedures for collecting antibody samples and producing plasma pools are provide, for example, in: U.S. Pat. Nos. 4,174,388; 4,346,073; 4,482,483; 4,587,121; 4,617,379; 4,659,563; 4,665,159; 4,717,564; 4,717,766; 4,801,450; 4,863,730; 5,505,945; 5,582,827; 6,692,739; 6,962,700; 6,984,492; 7,045,131; 7,488,486; 7,597,891; 6,372,216; U.S. Patent App. No. 2003/0118591; U.S. Patent App. No. 2003/0133929 U.S. Patent App. No. 2005/0053605; U.S. Patent App. No. 2005/0287146; U.S. Patent App. No. 2006/0110407; U.S. Patent App. No. 2006/0198848; U.S. Patent App. No. 2006/0222651; U.S. Patent App. No. 2007/0037170; U.S. Patent App. No. 2007/0249550; U.S. Patent App. No. 2009/0232798; U.S. Patent App. No. 2009/0269359; U.S. Patent App. No. 2010/0040601; U.S. Patent App. No. 2011/0059085; and U.S. Patent App. No. 2012/0121578; herein incorporated by reference in their entireties. Embodiments of the present disclosure may utilize any suitable combination of techniques, methods, or compositions from the above listed references.

In some embodiments, plasma and/or antibody samples are obtained from donor subjects in the form of donated or purchased biological material (e.g., blood or plasma). In some embodiments, plasma and/or antibody samples (e.g., blood, plasma, isolated antibodies, etc.) are obtained from a commercial source. In some embodiments, a plasma and/or antibody sample, blood donation, or plasma donation is screened for pathogens, and either cleaned or discarded if particular pathogens are present. In one embodiment, screening occurs prior to pooling a donor sample with other donor samples. In other embodiments, screening occurs after pooling of samples. Antibodies, blood, and/or plasma may be obtained from any suitable subjects. In some embodiments, antibodies, blood, and/or plasma are obtained from a subject who has recently (e.g., within 1 year, within 6 months, within 2 months, within 1 month, within 2 weeks, within 1 week, within 3 days, within 2 days, within 1 day) been vaccinated against or been exposed to one or more specific pathogens. In certain embodiments, a subject positive for antibodies to the pathogen of interest is administered antigens to that pathogen to increase titer of the desired antibodies. In some embodiments, a subject has produced antibodies and/or has elevated titer of antibodies against one or more specific pathogens. In certain embodiments, a subject, whether negative or positive for antibodies to a specific microbial pathogen is administered one or more different viral, bacterial and/or fungal antigens/vaccines in order to increase titer of specific, desired antibodies (e.g., viral-, bacterial- and/or fungal-specific antibodies). Pathogens to which a donor may have elevated titer of antibodies include, but are not limited to, respiratory syncytial virus (RSV) and coronavirus (e.g., coronavirus OC43, coronavirus 229E, coronavirus NL63, coronavirus HKU1, MERS-CoV, SARS-CoV, or SARS-CoV-2 (COVID-19)), or other human viral or bacterial pathogens.

In some embodiments, plasma samples known, identified, and/or selected (e.g., according to methods described herein) to contain elevated titer of a particular antibody (e.g., antibodies directed to coronavirus) or a set of plasma samples are combined (e.g., pooled) to produce a composition comprising pooled plasma samples (e.g., with elevated titer of antibodies directed to a particular pathogen or to a set of pathogens (e.g., coronavirus, and one or more other respiratory pathogens)). For example, a composition comprising pooled plasma samples is produced by pooling plasma samples obtained from selected human subjects, wherein the pooled plasma comprises elevated levels (e.g., elevated by about 20%, 30%, 40%, 50%, 60%, 70%, 85%, 90%, 100%, 125%, 150%, 160%, 170%, 175%, 180%, 200%, 225%, 250%, 275%, 300%, 350%, 400%, 450%, 500%, 550%, 600%, 650%, 700%, 750%, 800%, 850%, 900%, 950%, 1000% or more), compared to a control value (e.g., the pathogen-specific antibody titers found in a mixture of plasma samples obtained from 100 or more random human subjects), of pathogen-specific (e.g., coronavirus specific) antibody titers. In a further embodiment, immune globulin is prepared from the pooled plasma (e.g., according to techniques and methods described herein). In some embodiments, a composition comprising pooled plasma samples is produced by pooling plasma samples obtained from selected human donors and non-selected human donors, wherein the pooled plasma comprises elevated levels, compared to the pathogen-specific antibody titers found in a mixture of plasma samples obtained from 100 or more random human subjects, of coronavirus-specific antibody titers (e.g., individuals recently exposed to one or more of coronavirus OC43, coronavirus 229E, coronavirus NL63, coronavirus HKU1, MERS-CoV, SARS-CoV, or SARS-CoV-2 (COVID-19)), of coronavirus-specific antibody titers individuals recently vaccinated for coronavirus), and other viral pathogen specific titers. In one embodiment, a composition comprising pooled plasma samples and/or immune globulin prepared therefrom is a sterile solution with a pH of about 6.0-7.8 (e.g., 5.0-6.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or higher). In another embodiment, a composition comprising pooled plasma samples and/or immune globulin prepared therefrom is prepared according US FDA standards for immune globulin preparation (e.g., 37 CFR §§ 640.100; 640.101; 640.102; 640.103; 640.104, Apr. 1, 2013).

In one embodiment, a composition comprising pooled plasma samples and/or immune globulin prepared therefrom comprises elevated antibody titer levels, compared to a control antibody titer value (e.g., the pathogen-specific antibody titer found in a mixture of plasma samples obtained from 100 or more random human subjects), of pathogen-specific antibodies to respiratory syncytial virus and one or more respiratory pathogens selected from, influenza A virus, influenza B virus, parainfluenza virus type 1, parainfluenza virus type 2, metapneumovirus and coronavirus, wherein the elevated levels of RSV specific, influenza A virus specific, influenza B virus specific, parainfluenza virus type 1 specific, parainfluenza virus type 2 specific, metapneumovirus specific and/or coronavirus (e.g., coronavirus OC43, coronavirus 229E, coronavirus NL63, coronavirus HKU1, MERS-CoV, SARS-CoV, or SARS-CoV-2 (COVID-19) specific antibodies are elevated at least 20%, 30%, 40%, 50%, 60%, 70%, 85%, 90%, 100%, 125%, 150%, 175%, 200%, 225%, 250%, 275%, 300%, 350%, 400%, 450%, 500%, 550%, 600%, 650%, 700%, 750%, 800%, 850%, 900%, 950%, 1000% or more), compared to a control value (e.g., the pathogen-specific antibody titer level found in a mixture of plasma samples obtained from 100 or more random human subjects). The present disclosure provides a method, in one embodiment, of generating the above described composition comprising obtaining plasma samples from selected human donors and non-selected human donors; pooling 100 or more plasma samples from both selected donors and non-selected donors to generate the pooled plasma composition. In one embodiment, the plasma samples from selected human donors and non-selected human donors are screened in order to confirm the absence of bloodborne pathogens (e.g., before or after pooling). In a further embodiment, selected human donors are identified via identifying the specific titer of antibodies to one or more respiratory pathogens selected from respiratory syncytial virus, influenza A virus, influenza B virus, parainfluenza virus type 1, parainfluenza virus type 2, metapneumovirus and coronavirus (e.g., coronavirus OC43, coronavirus 229E, coronavirus NL63, coronavirus HKU1, MERS-CoV, SARS-CoV, or SARS-CoV-2 (COVID-19)). In a preferred embodiment, selected human donors are identified via identifying the specific titer of antibodies to respiratory syncytial virus and/or coronavirus. In a further embodiment, the selected human donors comprise high titer donors and medium titer donors, wherein high titer donors comprise a pathogen specific antibody titer that is 2-5 times, 5-8 times, 8-10 times, 10-14 times, 14 times or greater than a standard value (e.g., the titer of pathogen specific antibodies present in a pool of plasma samples from 100 or more random human subjects), and wherein medium titer donors comprise a pathogen specific antibody titer that is the titer of pathogen specific antibodies present in a pool of plasma samples from 100 or more random human subjects or that is only marginally higher (e.g., 5-20% higher) or marginally lower (e.g., 5-20% lower) than this value. In still a further embodiment, the selected human donors comprise high titer donors, medium titer donors and low titers donors, wherein high titer donors comprise a pathogen specific antibody titer that is 2-5 times, 5-8 times, 8-10 times, 10-14 times, 14 times or greater than a standard value (the titer of pathogen specific antibodies present in a pool of plasma samples from 100 or more random human subjects), wherein medium titer donors comprise a pathogen specific antibody titer that is the titer of pathogen specific antibodies present in a pool of plasma samples from 100 or more random human subjects or that is only marginally higher (e.g., 5-20% higher) or marginally lower (e.g., 5-20% lower) than this value, and wherein low titer donors comprise a pathogen specific antibody titer that is around 20-50 percent the titer of pathogen specific antibodies present in a pool of plasma samples from 100 or more random human subjects.

In one embodiment, identifying antibody titer comprises a plasma screening assay assessing neutralizing activity in a plasma sample, and screening assay assessing antibody titer. In one embodiment, neutralizing activity in plasma is measured via the absence of infection by coronavirus. In a further embodiment, the plasma screening assay assessing neutralization activity categorizes plasma samples as high titer, medium titer, or low titer for coronavirus specific antibodies (e.g., titer being calculated and assigned to a donor/donor sample at the dilution that give 50% inhibition of virus growth (that point which is 50% of the two extremes (saline plus virus is 100 growth and no virus added is 0 growth) according to methods described herein. In some embodiments, coronavirus antibody neutralization titers can range from at least 40 to about 30,000, In still a further embodiment, plasma samples identified as displaying the top 20%-30% of neutralizing activity of all donors are processed to produce purified immunoglobulin. In a preferred embodiment, only plasma samples identified as displaying the top 20% of neutralizing activity of all donors are processed to produce purified immunoglobulin. In one embodiment, a screening assay characterizes the coronavirus specific antibody titer of a purified immunoglobulin fraction of the plasma sample. In another embodiment, the pooled plasma composition comprises a coronavirus neutralization antibody titer of 100, 1000, 2000, 5000, 10000, 15000, 20000, 25000 or more. In one embodiment, the pooled plasma composition comprise about 1800-2500 liters (e.g., about 1000, about 1100, about 1200, about 1300, about 1400, about 1500, about 1600, about 1700, about 1800, about 1900, about 2000, about 2100, about 2200, about 2300, about 2400 or about 2500 liters) of plasma from 100 donors (e.g., with a coronavirus neutralization antibody titer of 40 or more). In one embodiment, a pooled plasma composition of the present disclosure comprises about 2200 liters of plasma (e.g., from 100 donors with a coronavirus neutralization antibody titer of 40 or more). The disclosure is not limited by the type of control utilized. In some embodiments, the control is plasma samples obtained from 100 or more random human subjects. In other embodiments, the control is a conventional hyperimmune immune globulin (e.g., hyperimmune immune globulin for rabies (HYPERRAB, Grifols, Clayton, N.C.), hyperimmune globulin for hepatitis (e.g., HYPERHEP B, Talecris Biotherapeutics, Research Triangle Park, N.C.), hyperimmune globulin for RSV (e.g., RESPIGAM, MEDIMMUNE, Inc.)). In still other embodiments, the control is any commercially available immunoglobulin (e.g., non-hyperimmune globulin).

In one embodiment, the pooled plasma composition comprises a coronavirus-specific antibody titer that is at least 2 fold greater (e.g., 2, 3, 4, 5, 6, 7, 8, 9, 10 fold or more) than the coronavirus-specific antibody titer found in a mixture of plasma samples obtained from 100 or more random human subjects. In one embodiment, the pooled plasma composition provides a therapeutic benefit to a subject administered the composition that is not achievable via administration of a mixture of plasma samples obtained from 100 or more random human subjects. Multiple types of therapeutic benefits are provided including, but not limited to, inhibition of infection caused by coronavirus or other respiratory, viral pathogen in a subject administered the composition for a duration of time that is longer than and not achievable in a subject administered a mixture of plasma samples obtained from 100 or more random human subjects; significant reduction in viral load in the lung and/or nose; significant reduction in lung histopathology; and/or significant reduction in the level of pathogenic viral RNA in lung, liver, kidney and/or other tissue.

In one embodiment, each individual plasma sample used in a process or composition of the present disclosure is collected only at an FDA approved blood establishments and is tested by serological tests (e.g., FDA approved serological tests). In another embodiment, an individual plasma sample and/or a pooled plasma composition of the present disclosure is tested for the presence of an infectious agent (e.g., viral pathogen) using Nucleic Acid Testing (NAT) and used in a process or composition of the present disclosure only when the absence of the pathogens is confirmed.

Embodiments of the present disclosure are not limited by the type of subject (e.g., mammal, non-human primate, human, etc.) administered or treated with a composition of the present disclosure (e.g., pooled plasma samples and/or immunotherapeutic composition comprising same). Indeed, the subject may be any subject in need of treatment with a composition of the present disclosure (e.g., a subject infected with or susceptible to infection (e.g., due to an immune deficiency) with an infectious agent (e.g., any one or more infectious agents described herein (e.g., respiratory pathogens))). In some embodiments, the subject is at elevated risk for infection (e.g., by one or multiple specific pathogens (e.g., respiratory pathogens)). The subject may be a neonate. In some embodiments, the subject has an immunodeficiency (e.g., a subject receiving immunosuppressing drugs (e.g., a transplant patient), suffering from a disease of the immune system, suffering from a disease that depresses immune functions, undergoing a therapy (e.g., chemotherapy) that results in a suppressed immune system, experiencing an extended hospital stay, and/or a subject anticipating direct exposure to a pathogen or pathogens. In some embodiments, the subject treated with the compositions and/or methods of the present disclosure include subjects with a healthy or normal immune system (e.g., that has a bacterial, viral and/or fungal infection). In some embodiments, the subject to be treated is one that has a greater than normal risk of being exposed to an agent or material (e.g., a toxin or toxins). In some embodiments, the subject is a soldier, an emergency responder or other subject that has a higher than normal risk of being exposed to a toxin (e.g., biological toxin), wherein treatment with the compositions and/or methods of the present disclosure provide the subject one or more immune response benefits (e.g., administration of an immunotherapeutic composition to a soldier prevents the soldier from showing signs or symptoms of disease or morbidity normally associated with exposure to a toxin).

The present disclosure thus provides methods and compositions for preventing and/or treating infections associated with viruses (e.g., coronaviruses). In some embodiments, the present disclosure provides compositions (e.g., kits) and methods for identifying subjects useful for providing donor plasma/serum (e.g., with high titers of viral-specific antibodies). In some embodiments, the present disclosure provides new therapeutic compositions for active and passive immunization against infections caused by and/or associated with a viral pathogens. In some embodiments, the present disclosure provides new therapeutic compositions for active and passive immunization against infections caused by and/or associated with a specific virus (e.g., respiratory syncytial virus or coronavirus).

### BRIEF DESCRIPTION OF DRAWINGS

FIGS. 1A-B: FIG. 1A: Representative chest X-ray from day 13 post-admission (day 2 post-treatment) of an adult patient with Severe Acute Respiratory Distress Syndrome and confirmed COVID-19. The patient displayed progression of confluent ground-glass consolidative opacities involving the lung bases, and involvement of the periphery of the upper lobes bilaterally. FIG. 1B: Chest X-ray from day 24 post-admission (day 13 post-treatment) display persistent bilateral patchy pulmonary infiltrates with improved pulmonary ventilation.

### DETAILED DESCRIPTION

Embodiments of the present disclosure relate to compositions and methods for the treatment and/or prevention of pathogenic infections (e.g., coronavirus infections). In particular, the present disclosure provides human plasma immunoglobulin compositions, methods of identifying human donors and donor samples for use in the compositions, and methods of utilizing the compositions for prophylactic administration and/or therapeutic treatment (e.g., passive immunization or immune-prophylaxis).

Hyperimmune serum globulins (immune serum globulin having high titers of a particular antibody), in distinction to normal immunoglobulin, have been therapeutically useful in treating patients who require immediate infusion of high titer antibodies. For example, tetanus hyperimmune globulin is useful in treating patients who may have suspected tetanus and rabies hyperimmune globulin for treating patients with suspected rabies. Hyperimmune serum globulins can be produced from plasma or serum obtained from a selected donor(s) who have elevated titers for a specific antibody than is normally found in the average population (that is not found at a high titer in the average population). These donors have either been recently immunized with a particular vaccine (See, e.g., U.S. Pat. No. 4,174,388) or else they have recently recovered from an infection or disease (See, e.g., Stiehm, Pediatrics, Vol. 63, No. 1, 301-319 (1979); herein incorporated by reference in its entirety). These high titer sera or plasmas are pooled and subjected to fractionation procedures (Cohn et al, J. Am. Chem. Soc., 68, 459 (1946); Oncley, et al, J. Am. Chem. Soc., 71, 541 (1949); herein incorporated by reference in their entireties). Such procedures have required specific selection of a donor or limited numbers of donors in order to produce hyperimmune globulin with elevated concentrations of the desired antibodies.

Coronaviruses are named for the crown-like spikes on their surface. There are four main sub-groupings of coronaviruses, known as alpha, beta, gamma, and delta. Human coronaviruses were first identified in the mid-1960s. The seven coronaviruses that can infect people are: 229E (alpha coronavirus;) NL63 (alpha coronavirus); OC43 (beta coronavirus); HKU1 (beta coronavirus); MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19). Coronaviruses are a large family of viruses that are common in people and many different species of animals, including camels, cattle, cats, and bats. Rarely, animal coronaviruses can infect people and then spread between people such as with MERS-CoV, SARS-CoV, and SARS-CoV-2 (COVID-19). The SARS-CoV-2 virus is a betacoronavirus, like MERS-CoV and SARS-CoV. All three of these viruses have their origins in bats. MERS-CoV and SARS-CoV have been known to cause severe illness in people. The complete clinical picture with regard to COVID-19 is not fully understood. Reported illnesses have ranged from mild to severe, including illness resulting in death. While information so far suggests that most COVID-19 illness is mild, a report out of China suggests serious illness occurs in 16% of cases. Older people and people with certain underlying health conditions like heart disease, lung disease and diabetes, for example, seem to be at greater risk of serious illness.

Respiratory syncytial virus (RSV) is considered the most important cause of severe respiratory disease in infants and young children. It can also be an important cause of lower respiratory tract disease in the elderly, hematopoietic stem cell transplant patients and organ transplant patients. In the United States alone it has been reported that this virus causes pneumonia, bronchitis and croup in approximately 4 million children each year, resulting in about 4500 deaths. In the western world it is the major cause for hospitalization of children (National Research Council News Report, 35, 9 (1985); Stott, E. J. et al, Archives of Virology, 84:1-52 (1985); and W. H. O. Scientific Group, World Health Organization Technical Report Series 642 (1980); herein incorporated by reference in their entireties).

In accordance with the embodiments provided herein, the present disclosure provides hyperimmune globulin compositions comprising pooled plasma samples and/or immunoglobulin prepared therefrom having increased neutralizing antibody titers against specific viral pathogens, such as for example, coronavirus (coronavirus OC43, coronavirus 229E, coronavirus NL63, coronavirus HKU1, MERS-CoV, SARS-CoV, SARS-CoV-2 (COVID-19)). As described further herein, the compositions include pooled plasma samples and/or immunoglobulin prepared therefrom, which are obtained from a plurality of donor human subjects (e.g., 100, 200, 300, 400, 500 or more subjects). In some embodiments, a pooled sample comprising higher neutralizing antibody titers against one virus also has proportionally higher neutralizing antibody titers against other viruses. For example, pooled plasma samples can be obtained from a plurality of donor human subjects having increased antibody titers against a coronavirus (e.g., at least 1.2 fold greater than antibody titers from a corresponding control sample or an antibody neutralization titer of at least 40 to about 30,000), and these pooled plasma samples can also have proportionally increased antibody titers against at least a second virus (e.g., at least 1.2 fold greater than antibody titers from a corresponding control sample), including, but not limited to, respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, and SARS-CoV-2 (COVID-19). Additionally, in some embodiments, pooled plasma samples can be obtained from a plurality of donor human subjects having increased antibody titers against RSV (e.g., at least 1.2 fold greater than antibody titers from a corresponding control sample or an antibody neutralization titer from at least 1000 to 8000), and these pooled plasma samples can also have proportionally increased antibody titers against a coronavirus (e.g., at least 1.2 fold greater than antibody titers from a corresponding control sample or an antibody neutralization titer from at least 40 to about 30,000).

In one embodiment, the pooled plasma composition comprises a coronavirus-specific antibody titer that is at least 1.2 fold greater (e.g., 1.2, 1.5, 2, 2.5, 3, 3.5, 4.5, 5, 6, 7, 8, 9, 10 fold or more) than the coronavirus-specific antibody titer found in a mixture of plasma samples obtained from a plurality of random human subjects.

The present disclosure provides novel hyperimmune globulin compositions (and methods of generating these compositions) containing high titers of coronavirus neutralizing antibodies, which are surprisingly and significantly different than conventional immune globulin preparations as well as other IVIG preparations (e.g., other hyperimmune IVIG preparations prepared from non-selected convalescent plasma from any recovered patient). In one embodiment, it was surprisingly discovered that hyperimmune globulin prepared according to methods of the present disclosure have an elevated titer of coronavirus neutralizing antibodies that are functionally reactive and independent of the total amount of binding antibodies (e.g., as measured by ELISA) that are present in the composition. In one preferred embodiment, the disclosure provides that total coronavirus IgG binding antibodies do not correlate with and are not predictive of the amount of functional, neutralizing antibodies present in immune globulin prepared from the sera/plasma of a convalescent patient and/o a vaccinated donor. For example, in some cases, low levels of binding antibody were associated with high levels of neutralizing antibodies, and vice versa. Thus, measurement of total antibody levels only in plasma and/or immunoglobulin preparations does not predict or correlate with protective efficacy of that preparation. Thus, in one embodiment, the present disclosure provides means for the identification and characterization of plasma and/or immune globulin compositions containing a desired functional, neutralizing coronavirus antibody titer rather than one in which only the total amount of IgG is known.

In accordance with these embodiments, anti-SARS CoV-2 antibody titer present in a donor plasma sample can be identified by total antibody binding (e.g., using an ELISA). For example, in some embodiments, the present disclosure provides a pooled plasma composition comprising plasma from a plurality of plasma donors wherein each donor's plasma exhibits an SARS CoV-2 antibody titer that is at least 1.2 fold greater (e.g., 1.2, 1.5, 2, 2.5, 3, 3.5, 4.5, 5, 6, 7, 8, 9, 10 fold or greater, or any value therebetween) than the SARS CoV-2-specific antibody titer found in a negative control (e.g., plasma devoid of coronavirus antibodies, or a mixture of plasma samples obtained from a plurality of random, non-convalescent human subjects). In some embodiments, plasma samples are selected based upon the total amount of SARS CoV-2-specific antibody titer (e.g., only those plasma samples that display a threshold (e.g., 2 fold or higher) SARS CoV-2-specific antibody titer are selected). In some embodiments, the selected plasma samples are assayed to characterize SARS CoV-2 neutralizing antibody titer in the samples. In further embodiments, plasma samples are selected based upon the SARS CoV-2 neutralizing antibody titer (e.g., only those plasma samples that display a SARS CoV-2 neutralizing antibody titer in the top 50%, 55%, 60%, 65%, 70%, 75%, 80%, 85%, 90% or higher of all plasma samples tested are selected). In some embodiments, plasma samples are selected based upon both the SARS CoV-2-specific antibody titer and the SARS CoV-2-specific neutralizing titer. For example, as disclosed herein, it was determined that of all convalescent, COVID-19 convalescent plasma analyzed, there exist plasma samples that display a high level of a SARS CoV-2-antibody binding, but lack a corresponding high SARS CoV-2-neutrlizing titer. Compositions and methods disclosed herein are useful at identifying these plasma samples in order to specifically exclude these plasma samples from use in a pooled plasma composition or immune globulin prepared therefrom disclosed herein. The disclosure is not limited to any particular assay for determining neutralizing antibody titer. Indeed, any assay available in the art may be utilized. In some embodiments, a plaque/focus reduction neutralization test (P/FRNT) is performed. In further embodiments, an automated high-throughput antibody neutralization assay based on foci and plaque reduction is used. In other embodiments, a virus reduction neutralization test (VRNT) is utilized (see, e.g., Whiteman et al., Am J Trop Med Hyg. 2018 December; 99(6):1430-1439). In still other embodiments, a pseudovirus neutralization assay is utilized (Creative Diagnostics, Shirley, N.Y.). In some embodiments, a multiplexed bead-based SARS-CoV-2 serological assay is used (Gaithersburg, Md.).

For example, in some embodiments, the disclosure provides a method of producing an immune globulin comprising obtaining a plurality of plasma samples from a plurality of plasma donors (e.g., COVID-19 convalescent plasma donors or COVID-19 vaccinated donors), conducting a first assay on each plasma sample to measure total anti-SARS CoV-2 antibody titer, selecting, based upon the first assay, plasma samples having a total anti-SARS CoV-2 antibody binding titer that is about two-fold or higher (e.g., 3-, 4-, 5-, 6-, 7-, 8-, 9-, 10-fold or higher) than the amount of total anti-SARS CoV-2 antibody binding titer in a control sample, conducting a second assay on each selected plasma sample from step (3) to measure SARS CoV-2 neutralizing antibody titer, identifying, based upon the second assay, plasma samples having a neutralizing antibody titer in the lower 65% of all plasma samples assayed and excluding the identified plasma samples from further processing, pooling the non-excluded plasma samples, and preparing immune globulin from the pooled plasma samples. In some embodiments, each of the plurality of plasma donors is a COVID-19 convalescent plasma donor. In other embodiments, each of the plurality of plasma donors is a COVID-19 vaccinated plasma donor. In some embodiments, the control sample is a mixture of plasma samples obtained from random human plasma donors (e.g., 50, 100, 150, 200, 250, 500, 1000 or more plasma donors or number therebetween). In other embodiments, the control sample is a commercially available immune globulin. In some embodiments, plasma samples having a neutralizing antibody titer in the lower 70% of all plasma samples assayed are identified and excluded from further processing. In still other embodiments, plasma samples having a neutralizing antibody titer in the lower 75% of all plasma samples assayed are identified and excluded from further processing. In other embodiments, plasma samples having a neutralizing antibody titer in the lower 80% of all plasma samples assayed are identified and excluded from further processing. The disclosure is not limited by the number of non-excluded, pooled plasma samples. In some embodiments, the number of non-excluded, pooled plasma samples is 250-500 or more. In some embodiments, the number of non-excluded, pooled plasma samples is 500-1000 or more. In some embodiments, the immune globulin is prepared using a cold alcohol fractionation process that isolates the immune globulin fraction from the pooled plasma as a solution. In further embodiments, the immune globulin is combined with a pharmaceutically acceptable carrier.

The disclosure further provides a method of providing immunotherapy to a subject in need thereof, comprising administering to the subject an immunotherapeutic composition comprising an immune globulin disclosed herein and a pharmaceutically acceptable carrier. In some embodiments, the immunotherapeutic composition is administered to the subject so as to provide from about 1.0-3.0 or more grams of immune globulin per kilogram of the subject. In some embodiments, each of the plurality of plasma donors is a COVID-19 convalescent plasma donor. In some embodiments, each of the plurality of plasma donors is a COVID-19 vaccinated plasma donor. In some embodiments, the immunotherapeutic composition further comprises a biologically active agent selected from the group consisting of an anti-inflammatory agent, an anti-cancer agent, an anti-microbial agent, an antihistamine, a cytokine, and a chemokine. In some embodiments, the immunotherapeutic composition further comprises an immunotherapeutic agent selected from the group consisting of a recombinant antibody, an antibody fragment, an antibody-like molecule, a monoclonal antibody, an antiviral, an immunotherapeutic protein and an immunotherapeutic small molecule. In some embodiments, the immunotherapeutic composition further comprises an anti-inflammatory agent selected from the group consisting of a recombinant antibody, an antibody fragment, a monoclonal antibody, an anti-inflammatory protein and an anti-inflammatory small molecule. In some embodiments, the subject treated is diagnosed with an infection or condition (e.g., a viral infection (e.g., SARS CoV-2 infection). In some embodiments, the subject is not diagnosed with an infection or condition. In some embodiments, the subject is age 65 or older.

The disclosure provides a pharmaceutical composition comprising an immune globulin disclosed herein. The disclosure provides a method of providing immunotherapy to a subject in need thereof, comprising administering to the subject a therapeutically effective amount of a pharmaceutical composition comprising an immune globulin disclosed herein. In some embodiments, the immunotherapy is used to treat and/or prevent infection in the subject. In some embodiments, the immunotherapy is used to treat and/or prevent inflammation in the subject. In some embodiments, the pharmaceutical composition further comprises an anti-toxin agent. In some embodiments, the anti-toxin agent is a mono-specific, bi-specific or multi-specific antibody with specificity toward a bacterial or fungal toxin. In some embodiments, the bacterial or fungal toxin is selected from the group consisting of Botulinum neurotoxin, Tetanus toxin, E. coli toxin, Clostridium difficile toxin, Vibrio RTX toxin, Staphylococcal toxins, Cyanobacteria toxin, and mycotoxins. In some embodiments, the pharmaceutical composition further comprises a biologically active agent selected from the group consisting of an anti-inflammatory agent, an anti-cancer agent, an anti-microbial agent, an antihistamine, a cytokine, and a chemokine. In some embodiments, the pharmaceutical composition further comprises an immunotherapeutic agent selected from the group consisting of a recombinant antibody, an antibody fragment, an antibody-like molecule, a monoclonal antibody, an antiviral, an immunotherapeutic protein and an immunotherapeutic small molecule. In some embodiments, the pharmaceutical composition further comprises an anti-inflammatory agent selected from the group consisting of a recombinant antibody, an antibody fragment, a monoclonal antibody, an anti-inflammatory protein and an anti-inflammatory small molecule. In some embodiments, the immunotherapy is used to treat coronavirus infection in the subject.

In further embodiments, the disclosure provides a method of treating a human patient comprising administering an immune globulin obtained by the disclosed methods to a subject/patient. In some embodiments, the immune globulin reduces viral load in the lung and/or nose of a subject administered the composition compared to a control subject not receiving the composition. In some embodiments, the immune globulin reduces lung histopathology of a subject administered the composition compared to a control subject not receiving the composition. In one embodiment, the pooled plasma composition provides a therapeutic benefit to a subject administered the composition that is not achievable via administration of a mixture of plasma samples obtained from a plurality of random human subjects. Embodiments of the present disclosure are not limited by the type of therapeutic benefit provided. Indeed, a variety of therapeutic benefits may be attained including those described herein. In one embodiment, the pooled plasma composition possesses enhanced viral neutralization properties compared to a mixture of plasma samples obtained from a plurality of random human subjects. In a further embodiment, the enhanced viral neutralization properties reduce and/or prevent infection in a subject administered the composition for a duration of time that is longer than, and not achievable in, a subject administered a mixture of plasma samples obtained from a plurality of random human subjects.

The methods and compositions of the present disclosure overcome hurdles of antibody-based therapeutics (e.g., immune globulin treatments). For example, compositions and methods of the disclosure overcome the risk of exacerbating COVID-19 severity via antibody-dependent enhancement (ADE). Although an understanding of a mechanism is not needed to practice the present disclosure, and while the disclosure is not limited to any particular mechanism, in one embodiment, the methods of compositions of the disclosure specifically identify and exclude plasma samples (e.g., prior to pooling plasma samples and immune globulin isolation) containing high levels of SARS CoV-2 specific antibodies that lack neutralizing antibodies or that contain antibodies at sub-neutralizing levels that bind to viral antigens without blocking or clearing infection.

ADE has been documented to increase the severity of multiple viral infections, including other respiratory viruses such as respiratory syncytial virus (RSV) (See, e.g., Kim et al., Am. J. Epidemiol. 89, 422-434 (1969); and Graham, Vaccine 34, 3535-3541 (2016)) and measles (See, e.g., Nader et al., J. Pediatr. 72, 22-28 (1968); and Polack, Pediatr. Res. 62, 111-115 (2007)). ADE in respiratory infections is included in a broader category named enhanced respiratory disease (ERD), which also includes non-antibody-based mechanisms such as cytokine cascades and cell-mediated immunopathology. ADE caused by enhanced viral replication has been observed for other viruses that infect macrophages, including dengue virus and feline infectious peritonitis virus (FIPV).

ADE has been documented to occur through two distinct mechanisms in viral infections: by enhanced antibody-mediated virus uptake into Fc gamma receptor IIa (FcγRIIa)-expressing phagocytic cells leading to increased viral infection and replication, or by excessive antibody Fc-mediated effector functions or immune complex formation causing enhanced inflammation and immunopathology. Both ADE pathways can occur when non-neutralizing antibodies or antibodies at sub-neutralizing levels bind to viral antigens without blocking or clearing infection.

ADE can be measured in several ways, including in vitro assays (which are most common for the first mechanism involving FcγRIIa-mediated enhancement of infection in phagocytes), immunopathology or lung pathology. ADE via FcγRIIa-mediated endocytosis into phagocytic cells can be observed in vitro and has been extensively studied for macrophage-tropic viruses, including dengue virus in humans. In this mechanism, non-neutralizing antibodies bind to the viral surface and traffic virions directly to macrophages, which then internalize the virions and become productively infected. Since many antibodies against different dengue serotypes are cross-reactive but non-neutralizing, secondary infections with heterologous strains can result in increased viral replication and more severe disease, leading to major safety risks. Non-neutralizing antibodies, or antibodies at sub-neutralizing levels, enhanced entry into alveolar and peritoneal macrophages, which are thought to disseminate infection and worsen disease outcome.

Accordingly, while an understanding of a mechanism is not needed to practice the present disclosure, and while the disclosure is not limited to any particular mechanism, in one embodiment, the methods of compositions of the disclosure provide pooled plasma compositions and immune globulin prepared therefrom that contain high titers of SARS CoV-2 neutralizing antibodies that prevent trafficking of virions to macrophages and infection of the macrophages, and/or that prevent secondary infections and/or that prevent viral entry into alveolar or peritoneal macrophages (e.g., thereby eliminating the risk of ADE or ERD).

Another described ADE mechanism is best exemplified by respiratory pathogens, Fc-mediated antibody effector functions can enhance respiratory disease by initiating a powerful immune cascade that results in observable lung pathology (See, e.g., Ye et al., Front. Immunol. 8, 317 (2017); and Winarski, et al., Proc. Natl Acad. Sci. USA 116, 15194-15199 (2019)). Fc-mediated activation of local and circulating innate immune cells such as monocytes, macrophages, neutrophils, dendritic cells and natural killer cells can lead to dysregulated immune activation despite their potential effectiveness at clearing virus-infected cells and debris. For non-macrophage tropic respiratory viruses such as RSV and measles, non-neutralizing antibodies have been shown to induce ADE and ERD by forming immune complexes that deposit into airway tissues and activate cytokine and complement pathways, resulting in inflammation, airway obstruction and, in severe cases, leading to acute respiratory distress syndrome.

Accordingly, while an understanding of a mechanism is not needed to practice the present disclosure, and while the disclosure is not limited to any particular mechanism, in one embodiment, the methods of compositions of the disclosure provide pooled plasma compositions and immune globulin prepared therefrom that contain high titers of SARS CoV-2 neutralizing antibodies that prevent activation of local and circulating innate immune cells (e.g., monocytes, macrophages, neutrophils, dendritic cells and natural killer cells) and prevent dysregulated immune activation, prevent immune cascades that results in observable lung pathology, prevent and/or reduce ADE and ERD by blocking deposit of immune complexes in airway tissue, and/or prevent inflammation, airway obstruction and, acute respiratory distress syndrome. Moreover, in some embodiments, the present disclosure provides methods of providing immunotherapy to a subject comprising administering to the subject an immunotherapeutic composition comprising an immune globulin of the disclosure such that the subject receives a high dose (e.g., from about 1.0-3 grams per kilogram, or any value therebetween) of the immune globulin (e.g., that acts as anti-inflammatory).

In one embodiment, identifying antibody titer comprises a plasma screening assay assessing neutralizing activity/titer (e.g., SARS CoV-2 neutralizing activity or other coronavirus neutralizing activity) in a plasma sample, and a screening assay assessing antibody titer (e.g., SARS CoV2-specific antibody titer and/or other coronavirus antibody titer). In one embodiment, neutralizing activity in plasma is measured via the absence of infection.

In another embodiment, the pooled plasma comprises elevated levels, compared to the pathogen-specific antibody titers found in a mixture of plasma samples obtained from 100 or more random human subjects, of pathogen-specific antibody titers to two, three, four or more respiratory pathogens described herein. In one embodiment, the pooled plasma comprises a coronavirus-specific antibody titer that is at least 1.2 fold greater (e.g. 1.2 fold, 1.4 fold, 1.6 fold, 1.8 fold, 2 fold, 3 fold, 4 fold, 5 fold 6 fold, 7 fold, 8 fold, 9 fold, 10 fold, 12 fold, 15 fold or more) than the coronavirus-specific antibody titer found in a mixture of plasma samples obtained from 100 or more random human subjects. In another embodiment, the pooled plasma comprises pathogen-specific antibody titers to at least two or more respiratory pathogens selected from respiratory syncytial virus, influenza A virus, influenza B virus, parainfluenza virus type 1, parainfluenza virus type 2, metapneumovirus, and coronavirus that are each elevated at least 1.2 fold compared to the pathogen-specific antibody titers found in a mixture of plasma samples obtained from 100 or more random human subjects. In another embodiment, the pooled plasma comprises pathogen-specific antibody titers to at least three or more respiratory pathogens selected from respiratory syncytial virus, influenza A virus, influenza B virus, parainfluenza virus type 1, parainfluenza virus type 2, metapneumovirus, and coronavirus that are each elevated at least 1.5 fold compared to the pathogen-specific antibody titers found in a mixture of plasma samples obtained from 100 or more random human subjects. In still another embodiment, the pooled plasma comprises pathogen-specific antibody titers to at least four or more respiratory pathogens selected from respiratory syncytial virus, influenza A virus, influenza B virus, parainfluenza virus type 1, parainfluenza virus type 2, metapneumovirus, and coronavirus that are each elevated at least 1.5 fold compared to the pathogen-specific antibody titers found in a mixture of plasma samples obtained from 100 or more random human subjects. In one embodiment, the pooled plasma comprises plasma samples obtained from 500-3000 or more (e.g., more than 100, 250, 500, 750, 1000, 1250, 1500, 1750, 2000, 2500, 3000, 3500, 4000 or more human subjects). In one embodiment, the pooled plasma is utilized to prepare immunoglobulin (e.g., for intravenous administration to a subject). In one embodiment, the pooled plasma and/or immunoglobulin provides a therapeutic benefit to a subject administered the pooled plasma and/or immunoglobulin that is not achievable via administration of a mixture of plasma samples (or immunoglobulin prepared from same) obtained from 100 or more random human subjects. Embodiments of the present disclosure are not limited by the type of therapeutic benefit provided. Indeed, a variety of therapeutic benefits may be attained including those described herein. In one embodiment, the pooled plasma and/or immunoglobulin possesses enhanced viral neutralization properties compared to a mixture of plasma samples obtained from 100 or more random human subjects or immunoglobulin prepared from same. For example, in one embodiment, the pooled plasma possesses enhanced viral neutralization properties against one or more (e.g., two, three, four, five or more) respiratory pathogens (e.g., described herein). In a further embodiment, the enhanced viral neutralization properties reduce and/or prevent infection in a subject administered the composition for a duration of time that is longer than, and not achievable in, a subject administered a mixture of plasma samples obtained from 100 or more random human subjects. In one embodiment, the pooled plasma and/or immunoglobulin prepared from same reduces the incidence of infection in a subject administered the composition. In another embodiment, a pooled plasma and/or immunoglobulin prepared from same reduces the number of days a subject administered the pooled plasma and/or immunoglobulin is required to be administered antibiotics (e.g., to treat infection). In yet another embodiment, a pooled plasma and/or immunoglobulin prepared from same increases the trough level of circulating anti-respiratory pathogen specific antibodies in a subject (e.g., increases the level of neutralizing titers specific for respiratory pathogens (e.g., thereby providing protective levels of anti-respiratory pathogen specific antibodies between scheduled dates of administration of the pooled plasma and/or immunoglobulin prepared from same that are not maintained in a subject administered a mixture of plasma samples obtained from 100 or more random human subjects or immunoglobulin prepared from same)). In one embodiment, the composition comprising pooled plasma samples further comprises a pharmaceutically acceptable carrier (e.g., any natural or non-naturally occurring carrier(s) known in the art). In one embodiment, a subject administered immunoglobulin prepared from pooled plasma according to the embodiments of the present disclosure displays a mean fold increase in anti-RSV neutralization titer that is at least 4 fold, at least 5 fold, at least 6 fold, at least 7 fold, at least 8 fold, at least 9 fold or more at a time point of at least 1 to 14 days post administration (e.g., 14 day, 15 days, 16 days, 17 days, 18 days, 19 days or more) of the immunoglobulin. Embodiments of the present disclosure are not limited by the amount of immunoglobulin administered to a subject. In one embodiment, a subject is administered between 100-5000 mg/kg of the immunoglobulin one time, or daily for two or more days (e.g., 2, 3, 4, or more consecutive days). In another embodiment, such doses are administered intermittently, e.g. every week, every two weeks, every three weeks, every four weeks, etc. In one embodiment, a subject is administered between 750-1500 mg/kg of immunoglobulin on day one and between 750-1500 mg/kg immunoglobulin on day 2. In one embodiment, a subject is administered 1500 mg/kg of immunoglobulin on day one and 750 mg/kg immunoglobulin on day 2. In another embodiment, a subject is administered 750 mg/kg of immunoglobulin on day one and 750 mg/kg immunoglobulin on day 2. In one embodiments, a subject is administered immunoglobulin on day one, optionally administered immunoglobulin on day 2, and then re-administered immunoglobulin every 21 days. In one embodiments, a subject is administered immunoglobulin on day one, optionally administered immunoglobulin on day 2, and then re-administered immunoglobulin every 28 days. In one embodiment, the pooled plasma and/or immunoglobulin prepared from same reduces the incidence of infection in a subject administered the composition. In another embodiment, a pooled plasma and/or immunoglobulin prepared from same reduces the number of days a subject administered the pooled plasma and/or immunoglobulin is required to be administered antibiotics (e.g., to treat infection).

In one embodiment, the composition comprising pooled plasma samples further comprises a pharmaceutically acceptable carrier (e.g., any natural or non-naturally occurring carrier(s) known in the art). In one embodiment, a subject administered immunoglobulin prepared from pooled plasma according to the embodiments of the present disclosure displays a mean fold increase in anti-coronavirus (e.g., coronavirus OC43, coronavirus 229E, coronavirus NL63, coronavirus HKU1, MERS-CoV, SARS-CoV, or SARS-CoV-2 (COVID-19)) neutralization titer that is at least 2 fold, at least 3 fold, at least 4 fold, at least 5 fold, at least 6 fold, at least 7 fold, at least 8 fold, at least 9 fold or more at a time point of at least 1 to 14 days post administration (e.g., 14 day, 15 days, 16 days, 17 days, 18 days, 19 days or more) of the immunoglobulin.

In certain embodiments, plasma and/or antibody samples comprise donated and/or purchased body fluid samples, for example individual blood or blood component samples (e.g., plasma). These samples may be purified and/or screened for the presence of pathogens or other impurities (e.g., before or after pooling). Multiple donor antibody samples (e.g., donor plasma samples or other antibody-containing samples) can pooled together to create a pooled plasma sample/primary antibody pool (e.g., after identifying or screening for desired antibody titer in the antibody samples). By combining individual antibody samples (e.g., blood or blood component (e.g., plasma) samples) which have higher than normal titers of antibodies to one or more selected antigens, epitopes, extracellular proteins, viral surface proteins, together with plasma taken from donors not selected for high titers, a pooled plasma sample/primary antibody pool is created that exhibits elevated titer for such antibodies. In some embodiments, selected antigens, epitopes, extracellular proteins, viral surface proteins, etc. are administered to subjects to induce the expression of desired antibodies (e.g., from which antibody samples can be harvested). The resulting enhanced high titer antibody sample (e.g., blood, serum, plasma, purified antibodies (e.g., containing higher antibody titer as compared to a control level (e.g., the antibody titer in pooled plasma samples from 100 or more random human subjects)) is recovered and pooled with antibody samples from other subjects exhibiting or anticipated to exhibit elevated titer for the same antibodies (or antibodies directed to the same antigens, extracellular proteins, viral surface proteins, etc.), or with antibody samples from subject that have not been screened for antibody titer or that possess a low or absent antibody titer to a specific pathogen. In some embodiments, the pooled antibody samples are purified, screened, and/or concentrated. In one embodiment, pooling of samples (e.g., 100 or more samples) occurs in a manner that uses the fewest possible number of samples from high titer donors (e.g., identified by the compositions and methods described herein) but that still maintains a desired, standardized and elevated antibody titer to one or more (e.g., two, three, four or more) respiratory pathogens described herein.

Certain embodiments of the present disclosure utilize plasma from subjects that have been administered immunogenic substances (e.g., vaccines, antigens, epitopes, extracellular proteins, viral surface proteins, etc.) in order to generate elevated levels of specific neutralizing antibodies within the subject. Embodiments of the present disclosure are not limited by the type of antigen used for administration to a subject (e.g., donor) to induce the expression of specific antibodies. In some embodiments, the antigen is a polysaccharide (e.g., unconjugated or conjugated to a carrier or protein) or a plurality of the same. In some embodiments, a vaccine is a commercially available vaccine. Embodiments of the present disclosure are not limited by the vaccine. Similarly, embodiments of the present disclosure are not limited by the type or route of administration/immunization. Indeed, any route/type of immunization may be utilized including, but not limited to, the methods described in U.S. Patent Publication Nos. US2008026002, US2007009542; US2002094338; US2005070876; US2002010428; US2009047353; US2008066739; and US2002038111), each of which is hereby incorporated by reference in its entirety. In some embodiments, the vaccine is a coronavirus vaccine (e.g., coronavirus mRNA vaccine (e.g., Moderna's mRNA-1273 vaccine (Moderna, Cambridge, Mass.) or adenovirus based vaccine (e.g., Astra Zeneca's AZD1222 (AstraZeneca, Gaithersburg, Mass.).

Thus, in some embodiments, the present disclosure provides methods of stimulating high antibody levels in a donor, which includes administering to an animal, for example a human, a pharmaceutically-acceptable composition comprising an immunologically effective amount of an antigen composition (e.g., a coronavirus antigen composition). The composition can include partially or significantly purified antigens (e.g., coronavirus antigens (e.g., polysaccharide, protein and/or peptide epitopes, obtained from natural or recombinant sources, which may be obtained naturally or either chemically synthesized, or alternatively produced in vitro from recombinant host cells expressing DNA segments encoding such epitopes)). Methods to determine the efficacy of immunization (e.g., determining the levels of coronavirus-specific antibody titers) are known in the art, and any known method may be utilized to assess the efficacy of immunization. In some embodiments, detection methods for the evaluation of the efficacy of a vaccine (e.g., a coronavirus conjugate vaccine) is used.

In some embodiments, kits and methods are provided that identify samples and/or pools with specific antibody titers (e.g., antibody titers that are elevated). In one embodiment, a suitable amount of a detection reagent (e.g., antibody specific for antibodies, an antigen, or other reagent known in the art) is immobilized on a solid support and labeled with a detectable agent. Antibodies can be immobilized to a variety of solid substrates by known methods. Suitable solid support substrates include materials having a membrane or coating supported by or attached to sticks, beads, cups, flat packs, or other solid support. Other solid substrates include cell culture plates, ELISA plates, tubes, and polymeric membranes. The antibodies can be labeled with a detectable agent such as a fluorochrome, a radioactive label, biotin, or another enzyme, such as horseradish peroxidase, alkaline phosphatase and 2-galactosidase. If the detection reagent is an enzyme, a means for detecting the detection reagent can be supplied with the kit. A suitable means for detecting a detectable agent employs an enzyme as a detectable agent and an enzyme substrate that changes color upon contact with the enzyme. The kit can also contain a means to evaluate the product of the assay, for example, a color chart, or numerical reference chart. Some suitable methods for characterizing samples and pools are provided in the references incorporated by reference herein. Embodiments of the present disclosure are not limited by the method used to characterize samples and pools as having elevated titer.

In certain embodiments, compositions are provided (e.g., antibody samples, pooled plasma samples, immunoglobulins, etc.) in which antibodies have been purified and/or isolated from one or more contaminants. Human immunoglobulins were first isolated on a large scale during the 1940's by F. J. Cohn. In some embodiments, the techniques provided by Cohn (Cohn et al., J. Am. Chem. Soc. 1946; 68:459-475; herein incorporated by reference in its entirety) or modified Cohn-techniques are utilized in preparation of immunoglobulins herein. In some embodiments, various purification and isolation methods are utilized to produce substantially unmodified, unaltered, non-denatured and/or native immunoglobulin molecules of high purity. Exemplary techniques are provided, for example, in U.S. Pat. No. 4,482,483, herein incorporated by reference in its entirety. In some embodiments, compositions (e.g., antibody pools) comprise >50% immunoglobulin (e.g., >60%, >70%, >80%, >90%, >95%, >99%). Various methods may be utilized for producing such compositions, including, for example, standard protein purification and isolation techniques as well as fractionation of biological fluids (e.g., plasma). Descriptions of fractionation of antibodies for use in immunotherapeutics are found, for example in U.S. Pat. No. 4,346,073 and other references provided herein, each of which is incorporated by reference in their entireties. In certain embodiments, immunoglobulins are purified by a fractional precipitation method, ion-exchange chromatography, size exclusion chromatography, ultrafiltration, affinity chromatography, or any suitable combinations thereof (See, e.g., U.S. Pat. Nos. 7,597,891; 4,256,631; 4,305,870; Lullau et al., J. Biol. Chem. 1996; 271:16300-16309; Corthesy, Biochem. Soc. Trans. 1997; 25:471-475; and Crottet et al., Biochem. J. 1999; 341:299-306; herein incorporated by reference in their entireties).

In some embodiments, plasma samples are pooled to produce a large volume of antibodies/immunoglobulins (e.g., for commercial, clinical, therapeutic, and/or research use). In particular embodiments, antibody samples (e.g., plasma samples) exhibiting a certain desired characteristic or characteristics are pooled to result in a primary antibody pool (e.g., pooled plasma samples) enhanced for, exhibiting, and/or enriched in that desired characteristic. In certain embodiments, antibody samples (e.g., plasma) obtained from a plurality of subjects (e.g., >2 subjects, >5 subjects, >10 subjects, >20 subjects, >100 subjects, >200 subjects, >500 subjects, >1,000 subjects, >2,000 subjects, >5,000 subjects, >10,000 subjects, or more) are pooled. The subjects from which the antibody samples (e.g., blood, plasma, etc.) may be obtained may have had recent exposure to a pathogen, antigen, or epitope, been recently vaccinated with a pathogen, antigen, or epitope, or have been specifically exposed to a pathogen, antigen, or epitope for the purpose of producing specific antibodies.

In some embodiments, methods are provided for pooling/combining primary antibody pools (e.g., pooled plasma samples) to produce secondary antibody pools or tailored antibody pools. Two or more primary antibody pools, each exhibiting a desired characteristic (e.g., antibodies against RSV, antibodies against coronavirus, etc.), are combined at a desired ratio to produce a tailored antibody pool. In some embodiments, a tailored antibody pool exhibits the relative sum of the characteristics of the primary antibody pools from which it is derived (e.g., tailored pool confers immunity to specific pathogens to an extent that is consistent with the relative amount of the primary pools from which it is derived). In other embodiments, a tailored antibody pool exhibits distinct characteristics from the primary antibody pools from which it is derived (e.g., tailored pool confers immunity to a specific pathogen to a greater extent than the primary pools from which it is derived used individually, provides enhanced general immunity compared to use of individual primary pools, provides enhanced anti-inflammatory benefit compared to use of individual primary pools).

A composition of the present disclosure (e.g., pooled plasma and/or immunoglobulin prepared from same) can be administered by any suitable means, including parenteral, subcutaneous, intraperitoneal, intrapulmonary, and, if desired for local treatment, intralesional administration. Parenteral infusions include intramuscular, intravenous, intraarterial, intraperitoneal, or subcutaneous administration. In addition, compositions of the present disclosure may be administered by pulse infusion, particularly with declining doses. Dosing can be by any suitable route, e.g. by injections, such as intravenous or subcutaneous injections, depending in part on whether the administration is acute or chronic.

A composition of the present disclosure may be formulated, dosed, and/or administered in a fashion consistent with good medical practice. Factors for consideration in this context include the particular disorder being treated, the clinical condition of the individual patient, the cause of the disorder, the site of delivery of the agent, the method of administration, the scheduling of administration, and other factors known to medical practitioners. Compositions of the present disclosure need not be, but optionally are formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a composition of the present disclosure (when used alone or in combination with one or more other additional therapeutic agents) may depend upon a number of factors including the type of disease to be treated, the type of antibody, the patient's size, body surface area, age, the particular compound to be administered, sex, time and route of administration, general health, interaction with other drugs being concurrently administered, the severity and course of the disease, whether the antibody is administered for preventive or therapeutic purposes, previous therapy, and the patient's clinical history.

An exact dosage may be determined by the individual physician in view of the patient to be treated. Dosage and administration are adjusted to provide sufficient levels of the active moiety (e.g., plasma pool) or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks, four weeks, six weeks, eight weeks or more, depending on half-life and clearance rate of the particular formulation.

A composition of the present disclosure may be administered to the patient at one time or over a series of treatments. Depending on the type and severity of the disease, about 1 μg/kg to 5000 mg/kg (e.g. 0.5 mg/kg-1500 mg/kg) of a composition of the present disclosure can be an initial candidate dosage for administration to the patient, whether, for example, by one or more separate administrations, or by continuous infusion. As described herein, additional drugs or agents (e.g., antibiotics, antivirals, anti-inflammatory and/or healing compounds) may be administered concurrently with a pooled plasma composition of the present disclosure. An exemplary daily dosage of such agent may range from about 1 μg/kg to 100 mg/kg or more. For repeated administrations over several days or longer, depending on the condition, the treatment can generally be sustained until a desired suppression of disease symptoms occurs. One exemplary dosage of a composition of the present disclosure would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to a patient. Such doses may be administered intermittently, e.g. every week or every two or three weeks. A medical practitioner is readily able to monitor the therapeutic administration of a composition of the present disclosure and can in turn determine if higher or lower doses of the composition is to be administered.

Compositions of the present disclosure may be administered (e.g., intravenously, orally, intramuscularly, subcutaneously, etc.) to a patient in a pharmaceutically acceptable carrier such as physiological saline. Such methods are well known to those of ordinary skill in the art.

Accordingly, in some embodiments of the present disclosure, a composition can be administered to a patient alone, or in combination with other drugs or in pharmaceutical compositions where it is mixed with excipient(s) or other pharmaceutically acceptable carriers. In one embodiment of the present disclosure, the pharmaceutically acceptable carrier is pharmaceutically inert. Depending on the condition being treated, pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, a composition of the present disclosure may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the compositions of the present disclosure (e.g., pharmaceutical compositions) can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in embodiments of the present disclosure include compositions wherein the active ingredients are contained in an effective amount to achieve the intended purpose. For example, an effective amount of a composition of the present disclosure may be that amount that results in the inhibition of growth and/or killing of viruses (e.g., coronavirus) in a subject. Determination of effective amounts is well within the capability of those skilled in the art, especially in light of the disclosure provided herein.

In addition to the active ingredients pharmaceutical compositions may contain suitable pharmaceutically acceptable carriers comprising excipients and auxiliaries that facilitate processing of the compositions of the present disclosure into preparations which can be used pharmaceutically.

The pharmaceutical compositions of embodiments of the present disclosure may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the compositions in water-soluble form. Additionally, suspensions of the compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compositions to allow for the preparation of highly concentrated solutions.

Compositions of the present disclosure formulated in a pharmaceutical acceptable carrier may be prepared, placed in an appropriate container, and labeled for treatment of an indicated condition. Conditions indicated on the label may include treatment or prevention of a viral infection.

The pharmaceutical composition may be provided as a salt and can be formed with many acids, including but not limited to hydrochloric, sulfuric, acetic, lactic, tartaric, malic, succinic, and the like. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-% mannitol at a pH range of 4.5 to 5.5 that is combined with buffer prior to use.

Compositions may optionally contain carriers such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the immunoglobulins can be brought about by the use of agents delaying absorption, for example, aluminum monostearate and gelatin.

In some embodiments, a composition of the present disclosure is administered to a subject to provide therapeutic, preventative, prophylactic, and/or other benefits.

In some embodiments, an immunotherapeutic composition of the present disclosure is effective in treating (e.g., therapeutically, preventatively, prophylactically, etc.) a subject and/or bind antigens from, and/or are directed to pathogenic viruses including, but not limited to: adenovirus, coxsackie virus, Epstein-barr virus, BK virus, hepatitis a virus, hepatitis b virus, hepatitis c virus, herpes simplex virus (type 1), herpes simplex virus (type 2), cytomegalovirus, human herpesvirus (type 8), human immunodeficiency virus (HIV), influenza virus, measles virus, mumps virus, human papillomavirus, parainfluenza virus, poliovirus, rabies virus, respiratory syncytial virus, rubella virus, and varicella-zoster virus.

The use of specific compositions and methods of the present disclosure to treat pathogens or treat/prevent infection may vary depending on the site of infection. For example, immunotherapeutic compositions used for treating and/or preventing respiratory infections (e.g., caused by or associated with coronavirus infection) might include immunoglobulins with antibodies and/or monoclonal antibodies specific for at least two of the following pathogens: respiratory syncytial virus, influenza A virus, influenza B virus, influenza C virus, parainfluenza virus type 1, parainfluenza virus type 2, rhinovirus, metapneumovirus, coronavirus, or any other respiratory or other type of pathogen known by those of ordinary skill in the art or described herein.

Various diseases (e.g., cancer, AIDS, etc.), infections, and treatments (e.g., antivirals, antirejections medications, chemotherapies, etc.) can result in localized or general inflammation in a subject, which can lead to discomfort, downstream health problems, morbidity, and/or death. In some embodiments, compositions and methods of the present disclosure provide anti-inflammatory benefits when administered to a subject. Pooled immunoglobulins have been shown to provide an anti-inflammatory action when passively administered (See, e.g., Nimmerjahn and Ravetch, Annu. Rev. Immunol. 2008. 26:513-33; Ramakrishna et al. Plos Pathogens. 2011. 7:6:e1002071; herein incorporated by reference in their entireties). In some embodiments, a composition of the present disclosure exerts enhanced anti-inflammatory effect (e.g., 10% enhancement, 20% enhancement, 50% enhancement, 2-fold enhancement 3-fold enhancement, 5-fold enhancement, 10-fold enhancement, or greater) compared to the anti-inflammatory effect of a mixture of plasma samples obtained from random human subjects (e.g., 1000 or more random human subjects). Although an understanding of a mechanism is not necessary to practice embodiments of the present disclosure and while these embodiments are not limited to any particular mechanism, in one embodiment, a pooled plasma composition of the present disclosure displays significantly enhanced anti-inflammatory effect compared to a conventional IVIG because the pooled plasma composition of the present disclosure comprises plasma from at least 100 donors (e.g., compared to a conventional hyperimmune globulin prepared from a limited number of donors (e.g., in one embodiment, the larger the number of different plasma samples pooled, the more beneficial the anti-inflammatory effect (e.g., the greater the histopathological benefit (e.g., reduction of epithelial cell death)) observed)) and/or because the pooled plasma composition is produced to exclude plasma samples that contain high levels of SARS CoV-2 antibody binding but that lack a corresponding high level of SARS CoV-2 neutralization activity.

In some embodiments, immunotherapeutic compositions of the present disclosure comprise specific antibody titers against specific pathogens. For example, the antibody titers for specific pathogens in the compositions of the present disclosure may be between 1 and 1000 μg/ml (e.g., 1 μg/ml . . . 2 μg/ml . . . 100 μg/ml . . . 200 μg/ml . . . 500 μg/ml . . . 1000 μg/ml), although higher and lower titers are contemplated.

In some embodiments, the protective activity of an immunotherapeutic composition disclosed is enhanced by further comprising one or more additional agents, including, but not limited to: antibiotics, antivirals, anti-inflammatory and/or healing compounds. For example, biocides, surfactants, bacterial blocking receptor analogues, cytokines, growth factors, macrophage chemotactic agents, cephalosporins, aminoglycosides, fluoroquinolones, etc., can be provided at therapeutically acceptable levels in the compositions of the present disclosure.

In some embodiments of the present disclosure, compositions are administered alone, while in other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Compositions of the present disclosure can be administered via any suitable route of administration (e.g., enteral route, parenteral route, etc.). The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. In typical embodiments, compositions are administered to a subject such that they enter the bloodstream (e.g., intravenous administration). In some embodiments, compositions are administered to devices or instruments that will come into contact with a subject's body (e.g., medical devices, bandages, etc.). The antibodies and compositions of the disclosure can be administered using any suitable method, such as by oral ingestion (e.g., pill, tablet, syrup, liquid, elixir, etc.), nasogastric tube, gastrostomy tube, injection (e.g., intravenous), infusion, implantable infusion pump, and osmotic pump. The suitable route and method of administration may vary depending on a number of factors such as the specific antibody or antibodies being used, the rate of absorption desired, specific formulation or dosage form used, type or severity of the disorder being treated, the specific site of action, and conditions of the patient, and can be readily selected by a person skilled in the art

The term “therapeutically effective amount” refers to an amount that is effective for an intended therapeutic purpose. For example, in the context of enhancing an immune response, a “therapeutically effective amount” is any amount that is effective in stimulating, evoking, increasing, improving, or augmenting any response of a mammal's immune system. In the context of providing anti-inflammatory action, a “therapeutically effective amount” is any amount that is sufficient to cause any desirable or beneficial reduction in inflammation or prevention of the occurrence of inflammation. The therapeutically effective amount of an antibody usually ranges from about 0.001 to about 5000 mg/kg, and more usually about 0.05 to about 100 mg/kg, of the body weight of the mammal. For example, the amount can be about 0.3 mg/kg, 1 mg/kg, 3 mg/kg, 5 mg/kg, 10 mg/kg, 50 mg/kg, or 100 mg/kg of body weight of the mammal. The precise dosage level to be administered can be readily determined by a person skilled in the art and will depend on a number of factors, such as the type, and severity of the disorder to be treated, the particular binding molecule employed, the route of administration, the time of administration, the duration of the treatment, the particular additional therapy employed, the age, sex, weight, condition, general health and prior medical history of the patient being treated, and like factors well known in the medical arts.

An immunotherapeutic composition or other composition of the present disclosure is often administered on multiple occasions. Intervals between single doses can be, for example, on the order of hours, days, weeks, months, or years. An exemplary treatment regimen entails administration once per week, once every two weeks, once every three weeks, once every four weeks, once a month, once every 3 months or once every three to 6 months. Example dosage regimens for a immunotherapeutic composition comprising a tailored antibody pool include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. Other dosages and regimens may be determined by clinicians, researchers, or other practitioners based on embodiments of the present disclosure.

Compositions of the present disclosure can be combined with additional agents (e.g., antibodies, antibody fragments, antibody-like molecules, monoclonal antibodies, or other proteins or small molecules) to enhance the immunotherapeutic and/or anti-inflammatory affect. Such additional agents may be produced recombinantly, synthetically, in vitro, etc. Embodiments of the present disclosure are not limited by the types of additional agents that a pooled plasma composition or other sample is combined with. In some embodiments, recombinant or synthetic antibodies (e.g., humanized monoclonals) or antibody fragments (e.g., directed to a specific pathogen or antigen) are added. In addition, antibodies (e.g., monoclonal, polyclonal, etc.) for specified viruses can be added to the compositions. In some embodiments, various therapeutics (e.g., anti-inflammatory agents, chemotherapeutics), stabilizers, buffers, etc. are added to the antibody sample pools, for example, to further enhance the efficacy, stability, administerability, duration of action, range of uses, etc.

In some embodiments, compositions of the present disclosure (e.g., pooled plasma samples and/or immunoglobulin prepared therefrom) are spiked with one or more antibodies that bind to one or more epitope(s) of a target antigen (e.g., epitope of a viral pathogen (e.g., an epitope of coronavirus)). The presence of one or more antibodies in the compositions described herein can enhance the therapeutic effects of the compositions, including treating and/or preventing one or more aspects of the viral infection. In some embodiments, the one or more antibodies bind a specific target antigen and may also have therapeutic efficacy against a given pathogen, such as a virus. In some embodiments, existing antibodies that can be added to the therapeutic compositions of the present disclosure to enhance therapeutic efficacy include, but are not limited to, antibodies that bind one or more antigenic regions of a virus that are conserved among viruses or viral subtypes, that are unique among viruses or viral subtypes (e.g., variants), and/or are present in a particular virus because of genetic recombination. For example, viral spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to gain entry into cells and establish infection is mediated by the interactions of its Spike glycoproteins with human cell surface receptors. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The 51 subunit of the ectodomain mediates binding of the virion to host cell-surface receptors through its receptor-binding domain (RBD). The S2 subunit fuses with both host and viral membranes, by undergoing structural changes.

In some embodiments, antibodies added to the compositions of the present disclosure (e.g., pooled plasma samples and/or immunoglobulin prepared therefrom) include antibodies targeting coronavirus Spike proteins. For example, the SARS-CoV-specific human monoclonal antibody, CR3022 (and/or variations and derivatives thereof), are added to the compositions of the present disclosure (see, e.g., Tian, X. et al., Emerg Microbes Infect. 2020 December; 9(1):382-385). In some embodiments, antibodies added to the compositions of the present disclosure include antibodies that bind one or more epitopes of coronavirus Spike proteins having amino acid sequences that are unique compared to other coronaviruses. For example, antibodies can be included that bind at least one amino acid variant at positions 455, 486, 493, 494, 501, and 505 of the 51 subunit of coronaviruses. Antibodies can also be included that bind at least one amino acid variant at positions 673, 678, and 686 of the S2 subunit of coronaviruses (see, e.g., Andersen, K. G. et al., Nature Medicine. Mar. 17, 2020). In other embodiments, antibodies can be included that bind one or more epitopes of coronavirus proteins that are not known to be naturally present (e.g., not endogenous to a coronavirus), but may have become part of a coronavirus genome due to genetic recombination or engineering. For example, antibodies can be included that bind one or more epitopes of HIV gp120 and/or Gag proteins.

As would be recognized by one of ordinary skill in the art based on the present disclosure, antibodies that bind one or more epitopes of a viral pathogen can be generated and added to the compositions of the present disclosure (e.g., pooled plasma samples). In some embodiments, antibodies can be generated against one or more epitopes of a coronavirus antigen (e.g., coronavirus OC43, coronavirus 229E, coronavirus NL63, coronavirus HKU1, MERS-CoV, SARS-CoV, or SARS-CoV-2 (COVID-19)). In accordance with these embodiments, the present disclosure includes any methods for generating a coronavirus antibody that binds at least one epitope of a coronavirus antigen. Such antibodies can be generated using amino acid sequence information currently available corresponding to any of the known coronavirus strains, as well as that of any future coronavirus strains identified, by methods known in the art, examples of which are described further below.

In some embodiments, antibodies can be generated that bind an epitope or epitopes present in more than one coronavirus strain (e.g., antibodies that recognize a conserved region of a coronavirus protein). In some embodiments, antibodies can be generated that bind an epitope or epitopes present in a single coronavirus strain (e.g., antibodies that recognize a unique region of a coronavirus protein). In accordance with these embodiments, the sequence of SARS-CoV-2 can be accessed via NCBI GenBank accession code MN908947 (SEQ ID NO: 1); the sequence of SARS-CoV can be accessed via NCBI GenBank accession code AY274119 (SEQ ID NO: 2); the sequence of MERS-CoV can be accessed via NCBI GenBank accession code NC_019843 (SEQ ID NO: 3); the sequence of HKU1 (beta coronavirus) can be accessed via NCBI GenBank accession code KF686346 (SEQ ID NO: 4); the sequence of OC43 (beta coronavirus) can be accessed via NCBI GenBank accession code NC 006213 (SEQ ID NO: 5); the sequence of NL63 (alpha coronavirus) can be accessed via NCBI GenBank accession code NC_005831 (SEQ ID NO: 6); and the sequence of 229E (alpha coronavirus) can be accessed via NCBI GenBank accession code NC_002645 (SEQ ID NO: 7).

In some embodiments, the present disclosure provides a coronavirus antigen, epitope, and any fragment thereof useful for generating an immunogenic response in a subject (e.g., a coronavirus vaccine composition), and using that subject as a plasma donor to generate immune globulin compositions, as described further herein. In some embodiments, the present disclosure includes human plasma immunoglobulin compositions containing antibodies specific for a coronavirus or coronaviruses obtained from human donor samples that have been immunized with a coronavirus vaccine and methods of utilizing the compositions for prophylactic administration and/or therapeutic treatment (e.g., passive immunization or immune-prophylaxis). For example, in some embodiments, antigens and epitopes from SARS-CoV-2 (COVID-19) can be identified based on sequence similarities to epitopes identified other coronaviruses, such as SARS-CoV (e.g., using Immune Epitope Database and Analysis Resource (IEDB), as described in Grifoni, A. et al., Cell Press (2020): https://marlin-prod.literatumonline.com/pb-assets/journals/research/cell-host-microbe/PDFs/CHOM_2264_S50.pdf). Table 1 provides SARS-CoV-2 (COVID-19) B cell epitope regions, and Table 2 provides SARS-CoV-2 (COVID-19) T cell epitope regions (below), based on sequence similarities to SARS-CoV (S=surface glycoprotein; M=membrane protein; N=nucleocapsid phosphoprotein).

TABLE 1

| | | | SARS-CoV-2 | SEQ | | |
|---|---|---|---|---|---|---|
| | SARS-CoV | SEQ ID | Sequence | ID | SARS-CoV-2 Region | % |
| Protein | Sequence | NO: | (COVID-19) | NO: | (Start-End) | Identity |
| S | DAVDCSQNPLAE | 8 | DAVDCALDPLSE | 9 | 287-317 | 69 |
| | LKCSVKSFEIDKG | | TKCTLKSFTVEK | | | |
| | IYQTSNF | | GIYQTSN | | | |
| S | VCGPKLSTDLIKN | 10 | VCGPKKSTNLVK | 11 | 524-598 | 80 |
| | QCVNFNFNGLTG | | NKCVNFNFNGLT | | | |
| | TGVLTPSSKRFQP | | GTGVLTESNKKF | | | |
| | FQQFGRDVSDFT | | LPFQQFGRDIADT | | | |
| | DSVRDPKTSEILD | | TDAVRDPQTLEIL | | | |
| | ISPCSFGGVSVIT | | DITPCSFGGVSVI | | | |
| S | GTNASSEVAVLY | 12 | GTNTSNQVAVLY | 13 | 601-640 | 78 |
| | QDVNCTDVSTAI | | QDVNCTEVPVAI | | | |
| | HADQLTPAWRIY | | HADQLTPTWRVY | | | |
| | STGNN | | STGS | | | |
| S | FSQILPDPLKPTK | 14 | FSQILPDPSKPSK | 15 | 802-819 | 89 |
| | RSFIED | | RSFIE | | | |
| S | FGAGAALQIPFA | 16 | FGAGAALQIPFA | 17 | 888-909 | 100 |
| | MQMAYRFNGIG | | MQMAYRFNGI | | | |
| M | MADNGTITVEEL | 18 | MADSNGTITVEE | 19 | 1-24 | 92 |
| | KQLLEQWNLVIG | | LKKLLEQWNLVI | | | |
| M | PLMESELVIGAVII | 20 | PLLESELVIGAVIL | 21 | 132-151 | 90 |
| | RGHLRMA | | RGHLRI | | | |
| N | PQGLPNNTASWF | 22 | RPQGLPNNTASW | 23 | 42-62 | 95 |
| | TALTQHGKEE | | FTALTQHGK | | | |
| N | NNAATVLQLPQG | 24 | NNNAATVLQLPQ | 25 | 153-172 | 95 |
| | TTLPKGFYA | | GTTLPKGF | | | |
| N | KHIDAYKTFPPTE | 26 | NKHIDAYKTFPPT | 27 | 355-401 | 90 |
| | PKKDKKKKTDEA | | EPKKDKKKKTDE | | | |
| | QPLPQRQKKQPT | | AQPLPQRQKKQP | | | |
| | VTLLPAADMDD | | TVTLLPAADM | | | |

TABLE 2

| | | | SARS-CoV-2 | | | |
|---|---|---|---|---|---|---|
| | SARS-CoV | SEQ ID | Sequence | SEQ ID | SARS-CoV-2 Region | % |
| Protein | Sequence | NO: | (COVID-19) | NO: | (Start-End) | Identity |
| S | VRGWVFGSTMN | 28 | IRGWIFGTTLDS | 29 | 101-118 | 50 |
| | NKSQSVI | | KTQSLL | | | |
| S | CTFEYISDAFSL | 30 | CTFEYVSQPFLM | 31 | 166-178 | 62 |
| | D | | D | | | |
| S | DAFSLDVSEKSG | 32 | QPFLMDLEGKQ | 33 | 173-185 | 38 |
| | N | | GN | | | |
| S | TNFRAILTAFSP | 34 | TRFQTLLALHRS | 35 | 236-258 | 17 |
| | AQDIW | | YLTPGDSSSGW | | | |
| S | KSFEIDKGIYQTS | 36 | KSFTVEKGIYQT | 37 | 304-321 | 78 |
| | NFRVV | | SNFRVQ | | | |
| S | STFFSTFKCYGV | 38 | SASFSTFKCYGV | 39 | 371-387 | 82 |
| | SATKL | | SPTKL | | | |
| S | KLPDDFMGCV | 40 | KLPDDFTGCV | 41 | 424-433 | 90 |
| S | NIDATSTGNYNY | 42 | NLDSKVGGNYN | 43 | 440-457 | 56 |
| | KYRYLR | | YLYRLFR | | | |
| S | YLRHGKLRPFER | 44 | YLYRLFRKSNLK | 45 | 451-468 | 58 |
| | DISNVP | | PFERDI | | | |
| S | RPFERDISNVPFS | 46 | KPFERDISTEIYQ | 47 | 462-474 | 54 |
| S | KSIVAYTMSLGA | 48 | QSIIAYTMSLGA | 49 | 690-707 | 72 |
| | DSSIAY | | ENSVAY | | | |
| S | SIVAYTMSL | 50 | SIIAYTMSL | 51 | 691-699 | 89 |
| S | TECANLLLQYGS | 52 | TECSNLLLQYGS | 53 | 747-763 | 94 |
| | FCTQL | | FCTQL | | | |
| S | VKQMYKTPTLK | 54 | VKQIYKTPPIKD | 55 | 785-802 | 78 |
| | YFGGFNF | | FGGFNF | | | |
| S | ESLTTTSTALGK | 56 | DSLSSTASALGK | 57 | 936-952 | 71 |
| | LQDVV | | LQDVV | | | |
| S | ALNTLVKQL | 58 | ALNTLVKQL | 59 | 958-966 | 100 |
| S | VLNDILSRL | 60 | VLNDILSRL | 61 | 976-984 | 100 |
| S | LITGRLQSL | 62 | LITGRLQSL | 63 | 996-1004 | 100 |
| S | QLIRAAEIRASA | 64 | QLIRAAEIRASA | 65 | 1011-1028 | 100 |
| | NLAATK | | NLAATK | | | |
| S | SWFITQRNFFSP | 66 | HWFVTQRNFYE | 67 | 1101-1115 | 73 |
| | QII | | PQII | | | |
| S | RLNEVAKNL | 68 | RLNEVAKNL | 69 | 1185-1193 | 100 |
| S | NLNESLIDL | 70 | NLNESLIDL | 71 | 1192-1200 | 100 |
| S | FIAGLIAIV | 72 | FIAGLIAIV | 73 | 1220-1228 | 100 |
| Orf 3a | RFFTLGSITAQP | 74 | RIFTIGTVTLKQ | 75 | 6-20 | 40 |
| | VKI | | GEI | | | |
| Orf 3a | SITAQPVKI | 76 | TVTLKQGEI | 77 | 12-20 | 22 |
| M | TLACFVLAAV | 78 | TLACFVLAAV | 79 | 61-70 | 100 |
| M | GLMWLSYFV | 80 | GLMWLSYFI | 81 | 89-97 | 89 |
| M | HLRMAGHSL | 82 | HLRIAGHHL | 83 | 148-156 | 78 |
| N | ALNTPKDHI | 84 | ALNTPKDHI | 85 | 138-146 | 100 |
| N | LQLPQGTTL | 86 | LQLPQGTTL | 87 | 159-167 | 100 |
| N | GETALALLLL | 88 | GDAALALLLL | 89 | 215-224 | 80 |
| N | LALLLLDRL | 90 | LALLLLDRL | 91 | 219-227 | 100 |
| N | LLLDRLNQL | 92 | LLLDRLNQL | 93 | 222-230 | 100 |
| N | RLNQLESKV | 94 | RLNQLESKM | 95 | 226-234 | 89 |
| N | TKQYNVTQAF | 96 | TKAYNVTQAF | 97 | 265-274 | 90 |
| N | GMSRIGMEV | 98 | GMSRIGMEV | 99 | 316-324 | 100 |
| N | MEVTPSGTWL | 100 | MEVTPSGTWL | 101 | 322-331 | 100 |
| N | QFKDNVILL | 102 | NFKDQVILL | 103 | 345-353 | 78 |
| Orf 1ab | CLDAGINYV | 104 | CLEASFNYL | 105 | 2139-2147 | 56 |
| Orf 1ab | WLMWFIISI | 106 | WLMWLIINL | 107 | 2292-2300 | 67 |
| Orf 1ab | ILLLDQVLV | 108 | ILLLDQALV | 109 | 2498-2506 | 89 |
| Orf 1ab | LLCVLAALV | 110 | SACVLAAEC | 111 | 2840-2848 | 56 |
| Orf 1ab | ALSGVFCGV | 112 | SLPGVFCGV | 113 | 2942-2950 | 78 |
| Orf 1ab | TLMNVITLV | 114 | TLMNVLTLV | 115 | 3639-3647 | 89 |
| Orf 1ab | SMWALVISV | 116 | SMWALIISV | 117 | 3661-3669 | 89 |

In some embodiments, monoclonal antibodies can be made using the hybridoma method first described by Kohler et al., Nature, 256:495 (1975), and further described, e.g., in Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981), and Ni, Xiandai Mianyixue, 26(4):265-268 (2006) regarding human-human hybridomas. Additional methods include those described, for example, in U.S. Pat. No. 7,189,826 regarding production of monoclonal human natural IgM antibodies from hybridoma cell lines. Human hybridoma technology (Trioma technology) is described in Vollmers and Brandlein, Histology and Histopathology, 20(3):927-937 (2005) and Vollmers and Brandlein, Methods and Findings in Experimental and Clinical Pharmacology, 27(3):185-91 (2005).

For various other hybridoma techniques, see, e.g., US 2006/258841; US 2006/183887 (fully human antibodies), US 2006/059575; US 2005/287149; US 2005/100546; US 2005/026229; and U.S. Pat. Nos. 7,078,492 and 7,153,507. An exemplary protocol for producing monoclonal antibodies using the hybridoma method is described as follows. In one embodiment, a mouse or other appropriate host animal, such as a hamster, is immunized to elicit lymphocytes that produce or are capable of producing antibodies that will specifically bind to the protein used for immunization. Antibodies are raised in animals by multiple subcutaneous (sc) or intraperitoneal (ip) injections of a polypeptide comprising a coronavirus antigen or a fragment thereof, and an adjuvant, such as monophosphoryl lipid A (MPL)/trehalose dicrynomycolate (TDM) (Ribi Immunochem. Research, Inc., Hamilton, Mont.). A polypeptide comprising a coronavirus antigen or a fragment thereof may be prepared using methods well known in the art, such as recombinant methods, some of which are further described herein. Serum from immunized animals is assayed for anti-coronavirus antibodies, and booster immunizations are optionally administered. Lymphocytes from animals producing anti-coronavirus antibodies are isolated. Alternatively, lymphocytes may be immunized in vitro.

Lymphocytes are then fused with myeloma cells using a suitable fusing agent, such as polyethylene glycol, to form a hybridoma cell. See, e.g., Goding, Monoclonal Antibodies: Principles and Practice, pp. 59-103 (Academic Press, 1986). Myeloma cells may be used that fuse efficiently, support stable high-level production of antibody by the selected antibody-producing cells, and are sensitive to a medium such as HAT medium. Exemplary myeloma cells include, but are not limited to, murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Antibodies of the present disclosure can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. in Methods in Molecular Biology 178:1-37 (O'Brien et al., ed., Human Press, Totowa, N.J., 2001). For example, one method of generating antibodies of interest is through the use of a phage antibody library as described in Lee et al., J. Mol. Biol. (2004), 340(5):1073-93. Repertoires of VH and VL genes can be separately cloned by polymerase chain reaction (PCR) and recombined randomly in phage libraries, which can then be searched for antigen-binding clones as described in Winter et al., Ann. Rev. Immunol., 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J Mol. Biol., 227: 381-388 (1992).

In general, nucleic acids encoding antibody gene fragments are obtained from immune cells harvested from humans or animals. If a library biased in favor of anti-coronavirus clones is desired, a subject can be immunized with one or more epitopes of a coronavirus to generate an antibody response, and spleen cells and/or circulating B cells other peripheral blood lymphocytes (PBLs) are recovered for library construction. In some embodiments, a human antibody gene fragment library biased in favor of anti-coronavirus clones is obtained by generating an antibody response in transgenic mice carrying a functional human immunoglobulin gene array (and lacking a functional endogenous antibody production system) such that coronavirus immunization gives rise to B cells producing human antibodies against one or more epitopes of a coronavirus antigen.

Alternatively, the use of spleen cells and/or B cells or other PBLs from an unimmunized donor provides a better representation of the possible antibody repertoire, and also permits the construction of an antibody library using any animal (human or non-human) species in which coronavirus is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc. Nucleic acid molecules encoding antibody variable gene segments (including VH and VL segments) can be recovered from the cells of interest and amplified.

DNA encoding hybridoma-derived monoclonal antibodies or phage display Fv clones is readily isolated and sequenced using conventional procedures (e.g., by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol., 5: 256 (1993) and Pluckthun, Immunol. Revs, 130: 151 (1992).

DNA encoding the Fv clones can be combined with known DNA sequences encoding heavy chain and/or light chain constant regions (e.g., the appropriate DNA sequences can be obtained from Kabat et al.) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. An Fv clone derived from the variable domain DNA of one animal (such as human) species and then fused to constant region DNA of another animal species to form coding sequence(s) for “hybrid,” full length heavy chain and/or light chain is included in the definition of “chimeric” and “hybrid” antibody as used herein. In certain embodiments, an Fv clone derived from human variable DNA is fused to human constant region DNA to form coding sequence(s) for full- or partial-length human heavy and/or light chains.

DNA encoding anti-coronavirus antibody derived from a hybridoma can also be modified, for example, by substituting the coding sequence for human heavy- and light-chain constant domains in place of homologous murine sequences derived from the hybridoma clone (e.g. as in the method of Morrison et al., Proc. Natl. Acad. Sci. USA, 81: 6851-6855 (1984)). DNA encoding a hybridoma- or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the present disclosure.

Antibodies may also be produced using recombinant methods. For recombinant production of an anti-coronavirus antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

In some embodiments, one or more coronavirus antigens (e.g., comprising one or more antigenic epitopes of a coronavirus antigen described herein) are used as or in a vaccine that is used to immunize a subject. In some embodiments, the subject can then be used as a plasma donor to generate immune globulin compositions, as described further herein. In some embodiments, the present disclosure includes human plasma immunoglobulin compositions containing antibodies specific for a coronavirus or coronaviruses obtained from human donor samples that have been immunized with a coronavirus vaccine and methods of utilizing the compositions for prophylactic administration and/or therapeutic treatment (e.g., passive immunization or immune-prophylaxis). Hyperimmune serum globulins (immune serum globulin having high titers of a particular coronavirus antibody), in distinction to normal immunoglobulin, have been therapeutically useful in treating patients who require immediate infusion of high titer antibodies. In some embodiments, hyperimmune globulin compositions of the present disclosure can be obtained from pooled plasma samples obtained from a plurality of donor human subjects (e.g., 50, 100, 200, 300, 400, 500 or more subjects) that have been immunized with one or more antigenic epitopes against a coronavirus (e.g., coronavirus OC43, coronavirus 229E, coronavirus NL63, coronavirus HKU1, MERS-CoV, SARS-CoV, or SARS-CoV-2 (COVID-19)).

In accordance with the embodiments disclosed herein, the compositions and methods of the present disclosure include the characterization and selection of donor human subjects with high antibody titers against a coronavirus (e.g., coronavirus OC43, coronavirus 229E, coronavirus NL63, coronavirus HKU1, MERS-CoV, SARS-CoV, or SARS-CoV-2 (COVID-19)). To aid in the characterization and selection of donor human plasma sufficient to be included in the therapeutic compositions of the present disclosure, various assays can be used to measure and/or quantify the total levels of antibody binding in a sample of donor plasma, as well as the levels of neutralizing antibodies in a sample of donor plasma. Suitable assays include flow cytometry assays, competitive assays, inhibition assays, immunofluorescence assays, enzyme-linked immunosorbent (ELISA) assays, lateral flow assays, sandwich assays, and neutralization assays. In some embodiments, characterization of a donor plasma sample is performed using an enzyme linked immunosorbent assay (ELISA). ELISA (also referred to in the art as an “enzyme immunoassay” (EIA)) is a plate-based assay technique designed for detecting and quantifying soluble substances such as peptides, proteins, antibodies, and hormones in a sample. In an ELISA, a target macromolecule (e.g., a cell receptor) is immobilized on a solid surface (e.g., a microplate) and then complexed with a binding member specific for the target (e.g., a ligand) that is linked to a reporter enzyme. Detection is accomplished by measuring the activity of the reporter enzyme via incubation with the appropriate substrate to produce a measurable product (e.g., absorbance, chemiluminescence, fluorescence, or other visual signal). In the context of the present disclosure, an ELISA may be performed in either competitive or non-competitive formats. In some embodiments, an ELISA can be used to measure total antibody binding to a given antigen in a donor plasma sample.

In some embodiments, the ELISA is a competitive inhibition assay (also referred to in the art as “inhibition ELISA” or “competitive immunoassay”) which enables the screening of inhibitory proteins by measuring the concentration of a potential inhibitor protein (e.g., a neutralizing antibody) by detection of signal interference. Systems and methods for performing ELISA are known in the art and commercially available (see, e.g., Methods in Immunodiagnosis, 2nd Edition, Rose and Bigazzi, eds., John Wiley and Sons, 1980 and Campbell et al., Methods of Immunology, W. A. Benjamin, Inc., 1964). Assays may be performed in the absence of cells or viruses (i.e., “cell-free” or “virus-free” assays).

In some embodiments, the disclosed methods desirably include positive and/or negative controls. A control may be analyzed concurrently with the sample from the subject, or a control may be analyzed before or after the sample has been analyzed using the disclosed methods. The results obtained from the sample can be compared to the results obtained from the control(s). Standard curves for the controls may be provided, with which assay results for the sample may be compared. Numerous neutralizing antibodies directed against several types of coronaviruses have been isolated, and any of these neutralizing antibodies may be included as a positive control panel. Exemplary coronavirus neutralizing antibodies that may be included in the positive control panel are disclosed in, for example, Zost et al., Rapid isolation and profiling of a diverse panel of human monoclonal antibodies targeting the SARS-CoV-2 spike protein. Nat Med (2020). In other embodiments, the method further comprises comparing the results obtained from the sample with the results obtained using a negative control. The negative control may comprise at least one antibody that does not neutralize coronavirus infection (i.e., “coronavirus non-neutralizing antibodies”). The negative control may comprise a panel of two or more, three or more, four or more, or at least five coronavirus non-neutralizing antibodies.

Embodiments of the present disclosure further provide methods of identifying plasma comprising coronavirus neutralizing antibodies. The disclosure is not limited to any particular assay for determining neutralizing antibody titer. Indeed, any assay available in the art may be utilized. In some embodiments, a plaque/focus reduction neutralization test (P/FRNT) is performed. In further embodiments, an automated high-throughput antibody neutralization assay based on foci and plaque reduction is used. In other embodiments, a virus reduction neutralization test (VRNT) is utilized (see, e.g., Whiteman et al., Am J Trop Med Hyg. 2018 December; 99(6):1430-1439). In still other embodiments, a pseudovirus neutralization assay is utilized (Creative Diagnostics, Shirley, N.Y.). In some embodiments, a multiplexed bead-based SARS-CoV-2 serological assay is used (Gaithersburg, Md.).

Descriptions of the plasma sample, solid support, conjugate, controls, and components thereof set forth above in connection with the methods of detecting coronavirus binding antibodies also are applicable to the methods of identifying plasma comprising coronavirus neutralizing antibodies. The disclosure also provides a method of identifying a subject (e.g., a subject that has been vaccinated with a vaccine specific for the coronavirus or a subject that has recovered from coronavirus infection) harboring coronavirus neutralizing antibodies and/or quantifying the titer of coronavirus neutralizing antibodies in a subject, the method comprising performing any of the above-described methods on a sample obtained from the subject (e.g., a sample comprising plasma).

The above-described methods may be utilized in a variety of applications, including to determine the efficacy of immunization or vaccination against a coronavirus (e.g., determining the levels of coronavirus-specific antibody titers). In such an embodiment, the disclosed methods are performed on a sample from a subject has been vaccinated with a vaccine specific for the coronavirus. The methods also may be employed to screen plasma for its ability to provide protection from coronavirus infection, as well as for therapeutic applications (e.g., convalescent plasma).

In some embodiments, the present disclosure provides methods for the characterization of donor plasma from individuals that have recovered from infection with SARS CoV-2, which contain anti-SARS CoV-2 antibodies. Plasma from coronavirus disease 2019 (COVID-19) convalescent patients can be analyzed for specific SARS CoV-2 antibody levels (e.g., using a serological assay such as an ELISA) as well as functional activity (using an assay to detect neutralizing anti-SARS CoV-2 activity).

### Definitions

As used herein, the term “subject” refers to any human or animal (e.g., non-human primate, rodent, feline, canine, bovine, porcine, equine, etc.).

As used herein, the term “sample” is used in its broadest sense and encompass materials obtained from any source. As used herein, the term “sample” is used to refer to materials obtained from a biological source, for example, obtained from animals (including humans), and encompasses any fluids, solids and tissues. In particular embodiments of the present disclosure, biological samples include blood and blood products such as plasma, serum and the like. However, these examples are not to be construed as limiting the types of samples that find use with the present disclosure.

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or V_{H}) and a heavy chain constant region. The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, CL. The constant regions of the antibodies may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of each heavy/light chain pair (V_{H} and V_{L}), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

As used herein, the term “antibody derivative” or “derivative” of an antibody refers to a molecule that is capable of binding to the same antigen that the antibody from which it is derived binds to and comprises an amino acid sequence that is the same or similar to the antibody linked to an additional molecular entity. The amino acid sequence of the antibody that is contained in the antibody derivative may be the full-length antibody, or may be any portion or portions of a full-length antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional entity by chemical coupling, genetic fusion, noncovalent association or otherwise. The term “antibody derivative” also encompasses chimeric antibodies, humanized antibodies, and molecules that are derived from modifications of the amino acid sequences of an antibody, such as conservation amino acid substitutions, additions, and insertions.

As used herein, the term “antigen” refers to any substance that is capable of inducing an adaptive immune response. An antigen may be whole cell (e.g. bacterial cell), virus, fungus, or an antigenic portion or component thereof. Examples of antigens include, but are not limited to, microbial pathogens, bacteria, viruses, proteins, glycoproteins, lipoproteins, peptides, glycopeptides, lipopeptides, toxoids, carbohydrates, tumor-specific antigens, and antigenic portions or components thereof.

As used herein, the term “antigen-binding fragment” of an antibody refers to one or more portions of a full-length antibody that retain the ability to bind to the same antigen that the antibody binds to.

As used herein, the terms “immunoglobulin,” “immunoglobulin molecule” and “IG” encompass (1) antibodies, (2) antigen-binding fragments of an antibody, and (3) derivatives of an antibody, each as defined herein. As described herein, immunoglobulin may be prepared from (e.g., fractionated from, isolated from, purified from, concentrated from, etc.) pooled plasma compositions (e.g., for administration to a subject). As used herein, the term “Intravenous immunoglobulin (IVIG)” refers to conventional immunoglobulin prepared from the plasma of over one thousand random human donors, whereas the term “IVIG of the present disclosure,” for example SARS CoV-2-IVIG described herein, and in particular, refers to immune globulin prepared from a plurality of human donors, according to methods of the present disclosure, that contains an elevated SARS CoV-2-specific antibody titer and neutralization titer compared to a control sample (e.g., conventional IVIG prepared from a mixture of plasma samples obtained from 100 or more random human plasma donors). Additionally, for example, coronavirus-IVIG described herein, and in particular, refers to immune globulin prepared from a plurality of human donors, according to methods of the present disclosure, that contains an elevated coronavirus specific antibody titer compared to a control sample (e.g., conventional IVIG prepared from a mixture of plasma samples obtained from 100 or more random human plasma donors). As used herein, the terms “hyperimmune globulin,” “hyperimmune serum globulin” and “hyperimmune immune globulin” refer to immune serum globulin having a high titer of antibodies specific for a single organism or antigen (e.g., specific for hepatitis, specific for tetanus, specific for rabies, or specific for varicella zoster) produced from plasma or serum obtained from a donor(s) that has an elevated antibody titer for the single, specific organism or antigen. For example, Varicella Zoster Immune Globulin (VZIG, Massachusetts Public Health Biologic Laboratories, Boston, Mass.; or VARIZIG, Cangene Corporation, Winnipeg, Canada)) is a purified human immune globulin that has a high antibody titer specific for varicella zoster prepared from several hundred plasma donors and lacks significant antibody titers, or has decreased antibody titers, for other organisms or antigens (e.g., measles). Other hyperimmune globulin products are generally produced from donors that have been immunized to the specific pathogen or antigen (e.g., Rabies Immune Globulin, HYPERRAB, Grifols, Clayton, N.C., produced from a few hundred or less donors immunized with rabies vaccine).

As used herein, the term “antibody sample” refers to an antibody-containing composition (e.g., fluid (e.g., plasma, blood, purified antibodies, blood or plasma fractions, blood or plasma components etc.)) taken from or provided by a donor (e.g., natural source) or obtained from a synthetic, recombinant, other in vitro source, or from a commercial source. The antibody sample may exhibit elevated titer of a particular antibody or set of antibodies based on the pathogenic/antigenic exposures (e.g., natural exposure or through vaccination) of the donor or the antibodies engineered to be produced in the synthetic, recombinant, or in vitro context. Herein, an antibody sample with elevated titer of antibody X is referred to as an “X-elevated antibody sample.” For example, an antibody sample with elevated titer of antibodies against cytomegalovirus is referred to as a “cytomegalovirus-elevated antibody sample.

As used herein, the term “isolated antibody” or “isolated binding molecule” refers to an antibody or binding molecule that is identified and separated from at least one contaminant with which it is ordinarily associated in its source. Examples of an isolated antibody include: an antibody that: (1) is not associated with one or more naturally associated components that accompany it in its natural state; (2) is substantially free of other proteins from its origin source; or (3) is expressed recombinantly, in vitro, or cell-free, or is produced synthetically and the is removed the environment in which it was produced.

As used herein, the terms “pooled plasma,” “pooled plasma samples” and “pooled plasma composition” refer to a mixture of two or more plasma samples and/or a composition prepared from same (e.g., immunoglobulin). Elevated titer of a particular antibody or set of antibodies in pooled plasma reflects the elevated titers of the antibody samples that make up the pooled plasma. For example, plasma samples may be obtained from subjects that have been vaccinated (e.g., with a vaccine) or that have naturally high titers of antibodies to one or more pathogens as compared to the antibody level(s) found in the population as a whole. Upon pooling of the plasma samples, a pooled plasma composition is produced (e.g., that has elevated titer of antibodies specific to a particular pathogen). Herein, a pooled plasma with elevated titer of antibody X (e.g., wherein “X” is a microbial pathogen) is referred to as “X-elevated antibody pool.” For example, a pooled plasma with elevated titer of antibodies against cytomegalovirus is referred to as “cytomegalovirus-elevated antibody pool.” Also used herein is the term “primary antibody pool” which refers to a mixture of two or more plasma samples. Elevated titer of a particular antibody or set of antibodies in a primary antibody pool reflects the elevated titers of the antibody samples that make up the primary antibody pool. For example, many plasma donations may be obtained from subjects that have been vaccinated (e.g., with a polyvalent Pseudomonas aeruginosa vaccine). Upon pooling of the plasma samples, a primary antibody pool is produced that has elevated titer of antibodies to Pseudomonas aeruginosa. Herein, a primary antibody pool with elevated titer of antibody X (e.g., wherein “X” is a microbial pathogen) is referred to as “X-elevated antibody pool.” For example, a primary antibody pool with elevated titer of antibodies against cytomegalovirus is referred to as “cytomegalovirus-elevated antibody pool.” Pooled plasma compositions can be used to prepare immunoglobulin (e.g., that is subsequently administered to a subject) via methods known in the art (e.g., fractionation, purification, isolation, etc.). The present disclosure provides that both pooled plasma compositions and immunoglobulin prepared from same may be administered to a subject to provide prophylactic and/or therapeutic benefits to the subject. Accordingly, the term pooled plasma composition may refer to immunoglobulin prepared from pooled plasma/pooled plasma samples.

As used herein, the term “secondary antibody pool” or “tailored antibody pool” refer to a mixture of two or more primary antibody pools. Such a pool for example, may be tailored to exhibit elevated titer of specific antibodies or sets of antibodies by combining primary pools that exhibit such elevated titers. For example, a primary pool with elevated titer of Pseudomonas aeruginosa antibodies could be combined with a primary pool with elevated titer of Varicella-zoster virus antibodies to produce a tailored antibody pool with elevated titer of antibodies against Pseudomonas aeruginosa and Varicella-zoster virus.

As used herein, the term, “spiked antibody pool” refers to a pooled plasma sample (e.g., primary or tailored antibody pool) that contains antibodies from at least one natural source spiked or combined with antibodies or other immunoglobulin produced synthetically, recombinantly, or through other in vitro means.

As used herein, the term “isolated antibody” or “isolated binding molecule” refers to an antibody or binding molecule that is identified and separated from at least one contaminant with which it is ordinarily associated in its source. Examples of an isolated antibody include: an antibody that: (1) is not associated with one or more naturally associated components that accompany it in its natural state; (2) is substantially free of other proteins from its origin source; or (3) is expressed recombinantly, in vitro, or cell-free, or is produced synthetically and the is removed the environment in which it was produced.

As used herein, the term “purified” or “to purify” means the result of any process that removes some of a contaminant from the component of interest, such as a protein (e.g., antibody) or nucleic acid. The percent of a purified component is thereby increased in the sample.

As used herein, the term “immunotherapeutic agents” refers to a chemical or biological substance that can enhance an immune response (e.g., specific or general) of a mammal. Examples of immunotherapeutic agents include: passively administered primary antibody pools; tailored antibody pools (e.g., passively administered tailored antibody pools); vaccines, chemokines, antibodies, antibody fragments, bacillus Calmette-Guerin (BCG); cytokines such as interferons; vaccines such as MyVax personalized immunotherapy, Onyvax-P, Oncophage, GRNVAC1, Favld, Provenge, GVAX, Lovaxin C, BiovaxID, GMXX, and NeuVax; and antibodies such as alemtuzumab (CAMPATH), bevacizumab (AVASTIN), cetuximab (ERBITUX), gemtuzunab ozogamicin (MYLOTARG), ibritumomab tiuxetan (ZEVALIN), panitumumab (VECTIBIX), rituximab (RITUXAN, MABTHERA), trastuzumab (HERCEPTIN), tositumomab (BEXXAR), tremelimumab, CAT-3888, agonist antibodies to CD40 receptor that are disclosed in WO2003/040170, and any immunomodulating substance.

As used herein, the term “donor” refers to a subject that provides a biological sample (e.g., blood, plasma, etc.). A donor/donor sample may be screened for the presence or absence of specific pathogens (e.g., using U.S. Food and Drug Administration (FDA) guidelines for assessing safety standards for blood products (e.g., issued by the FDA Blood Products Advisory Committee). For example, a donor/donor sample may be screened according to FDA guidelines to verify the absence of one or more bloodborne pathogens (e.g., human immunodeficiency virus (HIV) 1 (HIV-1), HIV-2; Treponema pallidum (syphilis); Plasmodium falciparum, P. malariae, P. ovale, P. vivax or P. knowlesi (malaria); hepatitis B virus (HBV), hepatitis C virus HCV); prions (Creutzfeldt Jakob disease); West Nile virus; parvovirus; Typanosoma cruzi; coronavirus (e.g., coronavirus OC43, coronavirus 229E, coronavirus NL63, coronavirus HKU1, MERS-CoV, SARS-CoV, or SARS-CoV-2 (COVID-19)); vaccinia virus or other pathogen routinely screened or that is recommended to be screed for by a regulatory body such as the FDA). As used herein, the terms “selected donor,” “selected human subject” and the like refer to a subject that is chosen and/or identified to provide a biological sample (e.g., blood, plasma, etc.) based on the presence of a desired characteristic of that biological sample (e.g., a specific titer (e.g., high, average or low titer) of antibodies (e.g., determined using one or more screening methods (e.g., neutralization assay or other assay (e.g., ELISA) described herein) specific for one or more pathogens (e.g., one or more respiratory pathogens (e.g., SARS CoV-2))). For example, in one embodiment described herein, a high titer selected donor comprises a pathogen specific antibody titer that is about 1.5-2.0 times, 2-5 times, 5-8 times, 8-10 times, 10-14 times, 14 times or greater than a standard value (the titer of pathogen specific antibodies present in a pool of plasma samples from 100 or more random human subjects), wherein medium titer donors comprise a pathogen specific antibody titer that is the titer of pathogen specific antibodies present in a pool of plasma samples from 100 or more random human subjects or that is only marginally higher (e.g., 5-20% higher) or marginally lower (e.g., 5-20% lower) than this value, and wherein low titer donors comprise a pathogen specific antibody titer that is around 20-50 percent or less than the titer of pathogen specific antibodies present in a pool of plasma samples from 100 or more random human subjects. Thus, a random donor/random donor sample may be a subject/sample that passes FDA bloodborne pathogen screening requirements and is not selected on the basis of antibody titers (e.g., SARS CoV-2 antibody titers).

As used herein, an “immunostimulatory amount” refers to that amount of a vaccine (e.g., viral, bacterial and/or fungal vaccine) that is able to stimulate the immune response. An immune response includes the set of biological effects leading to the body's production of immunoglobulins, or antibodies, in response to a foreign entity. Accordingly, immune response refers to the activation of B cells, in vivo or in culture, through stimulation of B cell surface Ig receptor molecules. The measurement of the immune response is within the ordinary skill of those in this art and includes the determination of antibody levels using methods described in the series by P. Tijssen, Laboratory Techniques in Biochemistry and Molecular Biology: Practice and Theory of Enzyme Immunoassays, (Burdon & van Knippenberg eds., 3rd ed., 1985) Elsevier, New York; and Antibodies: A Laboratory Manual, (Harlow & Lane eds., 1988), Cold Spring Harbor Laboratory Press; as well as procedures such as countercurrent immuno-electrophoresis (GIEP), radioimmunoassay, radio-immunoprecipitation, enzyme-linked immuno-sorbent assays (ELISA), dot blot assays, and sandwich assays, see U.S. Pat. Nos. 4,376,110 and 4,486,530, all of which are incorporated by reference. Measurement of the immune response also includes detection or determination of B cell activation events that may precede antibody production, or signal an increase in antibody production. Such measurements include, B cell proliferation assays, phosphorylation assays, assays of intracytoplasmic free calcium concentration, and other methods of determining B cell activation known in the art. Representative assays are provided in Mongini et al., J. Immunol. 159:3782-91 (1997); Frade, et al., BBRC 188:833-842 (1992); Tsokos et al., J. Immunol. 144:1640-1645 (1990); Delcayre et al., BBRC 159:1213-1220 (1989); and Nemerow et al., J. Immunol. 135:3068-73 (1985) each of which is incorporated by reference. In preferred embodiments, the practice of the present disclosure includes promoting, enhancing or stimulating an immune response. These actions refer to establishing an immune response that did not previously exist; to optimizing or increasing a desired immune response; to establishing or increasing a secondary response characterized by increased isotype switching, memory response, or both; to providing a statistically increased immunoprotective effect against a pathogen; to generating an equivalent or greater humoral immune response, or other measure of B cell activation, from a reduced or limiting dose of antigen; to generating an increased humoral immune response, or other measure of B cell activation, in response to an equivalent dose of antigen; or to lowering the affinity threshold for B cell activation in vivo or in vitro. Preferably, an immunostimulatory amount refers to that amount of vaccine that is able to stimulate an immune response in a subject (e.g., a donor), and from which subject plasma, serum or other blood component is harvested for use in the compositions and methods of the present disclosure (e.g., for the therapeutic and/or prophylactic treatment of microbial (e.g., viral, bacterial and/or fungal) infection in a subject treated with compositions and methods described herein)).

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the term “adjuvant” refers to any substance that can stimulate an immune response (e.g., a mucosal immune response). Some adjuvants can cause activation of a cell of the immune system (e.g., an adjuvant can cause an immune cell to produce and secrete a cytokine). Examples of adjuvants that can cause activation of a cell of the immune system include, but are not limited to, the nanoemulsion formulations described herein, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); cholera toxin (CT), and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions of the present disclosure are administered with one or more adjuvants (e.g., to skew the immune response towards a Th1 and/or Th2 type response). In some embodiments, an adjuvants described in US2005158329; US2009010964; US2004047882; or U.S. Pat. No. 6,262,029 (each of which is hereby incorporated by reference in its entirety) is utilized.

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

A used herein, the term “immune response” refers to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the terms “immunogen” and “antigen” refer to an agent (e.g., a microorganism (e.g., bacterium, virus or fungus) and/or portion or component thereof (e.g., a protein antigen or a polysaccharide)) that is capable of eliciting an immune response in a subject.

As used herein, the term “pathogen product” refers to any component or product derived from a pathogen including, but not limited to, polypeptides, peptides, proteins, nucleic acids, membrane fractions, and polysaccharides.

The terms “pharmaceutically acceptable” or “pharmacologically acceptable,” as used herein, refer to compositions that do not substantially produce adverse reactions (e.g., toxic, allergic or immunological reactions) when administered to a subject.

As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintrigrants (e.g., potato starch or sodium starch glycolate), polyethyl glycol, other natural and non-naturally occurring carries, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present disclosure that is physiologically tolerated in the target subject. “Salts” of the compositions of the present disclosure may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the present disclosure and their pharmaceutically acceptable acid addition salts. Examples of bases include, but are not limited to, alkali metal (e.g., sodium) hydroxides, alkaline earth metal (e.g., magnesium) hydroxides, ammonia, and compounds of formula NW4⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH4⁺, and NW4⁺ (wherein W is a C₁₋₄ alkyl group), and the like. For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compositions of the present disclosure are contemplated as being pharmaceutically acceptable. However, salts of acids and bases that are non-pharmaceutically acceptable may also find use, for example, in the preparation or purification of a pharmaceutically acceptable composition.

As used herein, the terms “at risk for infection” and “at risk for disease” refer to a subject that is predisposed to experiencing a particular infection or disease (e.g., respiratory infection or disease). This predisposition may be genetic (e.g., a particular genetic tendency to experience the disease, such as heritable disorders), or due to other factors (e.g., immunosuppression, compromised immune system, immunodeficiency, environmental conditions, exposures to detrimental compounds present in the environment, etc.). Thus, it is not intended that embodiments of the present disclosure be limited to any particular risk (e.g., a subject may be “at risk for disease” simply by being exposed to and interacting with other people), nor is it intended that embodiments of the present disclosure be limited to any particular diseaseThe present invention relates to a steam sample concentrator and conditioning (SSCC) system. The SSCC system concentrates impurities carried in steam and facilitates analysis of the impurities. The SSCC system prevents the dissolution of noncondensable gases (NCGs) (e.g., hydrogen sulfide (H₂S) and carbon dioxide (CO₂)) in geothermal steam that interfere with steam analysis. For example, when steam is analyzed, it is often separated into condensate and noncondensable gas phases. The SSCC system of the invention, via prevention of the dissolution of NCGs in a steam sample after separation of condensate and noncondensable gas phases, enables a significantly more accurate measurement of the impurities in the sample compared to conventional systems.

### EXAMPLES

The following examples are provided in order to demonstrate and further illustrate certain preferred embodiments and aspects of the present disclosure and are not to be construed as limiting the scope thereof.

Studies have shown that the ability of humans to respond to foreign antigens (e.g., microbial pathogens (e.g., naturally occurring or in the form of a vaccine)) is controlled by the major histocompatibility complex (human leukocyte antigen “HLA” type in humans, the major histocompatibility complex of the mouse, H-2, is homologous to HLA in humans). Additional studies have shown that the histocompatibility complex controls the humoral antibody responses generated within a subject against microbial pathogens. Different HLA typing programs have existed for some time and have studied HLA type with regard to various immunological responses in humans (e.g., bone marrow transplant graft and/or rejection, organ transplant, autoimmunity, cancer, and strength of immune response (e.g., humoral immune response) generated by a subject). While HLA typing can be useful in these limited contexts, cost, medical record and other concerns make it unfeasible to HLA type individuals in other contexts.

Experiments were conducted during development of embodiments of the present disclosure in order to determine if a subset of plasma donors could be identified as strong (e.g., high) humoral immune responders in the absence of HLA typing. For example, the identification of individual subjects, or a population of individuals, that are strong humoral immune responders may itself be useful in order to identify individuals as potential plasma donors (e.g., for the manufacture of immunoglobulin). In addition, experiments were conducted in order to determine if individuals could be identified that were strong responders not only to a single microbial pathogen but to a plurality of microbial pathogens (e.g., such an individual, or population of individuals, may contain high titers due to the strength of humoral immune response in the subject, not just to a single microbial pathogen/antigen but to a plurality of microbial pathogens/antigens (e.g., any or all of the microbial pathogens/antigens to which the individual, or population of individuals, had been exposed in the course of their lifetime(s)). Thus, experiments were conducted in an effort to identify plasma donors as generally high responders in the absence of having to tissue type (e.g., HLA type) the donors for a specific histocompatibility gene complex. To this end, plasma donor samples were studied and characterized for antibody titers for one or a plurality respiratory pathogens in order to characterize the donors (e.g., as a high/strong responder to antigen challenge via generation of elevated levels of antibodies versus donors that are not strong responders/do not generate elevated levels of antibodies (e.g., via determining antibody titers to one or more respiratory pathogens in the subjects)).

Respiratory pathogens were chosen because individuals are ubiquitously exposed to a plurality of respiratory pathogens. That is, almost all adult and pediatric human populations have been exposed to a plurality of respiratory pathogens and would have therefore generated at some point in their lifetime a humoral antibody response that is measurable. Experiments were conducted in order to determine if high/strong responders could be identified using antibody titers to one or a plurality of respiratory pathogens. In one non-limiting example described below, experiments were conducted in order to determine if antibody titer to respiratory syncytial virus (RSV) in a donor plasma sample could be used to predict the antibody titer to other respiratory pathogens in the donor plasma sample. For example, experiments were performed in order to determine if high antibody titer to a respiratory pathogen (e.g., RSV) could be used as a biomarker to identify a donor as an overall high/strong responder to antigen challenge (e.g., to a plurality of respiratory or other pathogens) via generation of elevated levels of antibodies, versus donors that are not strong responders/do not generate elevated levels of antibodies.

As shown in Tables 3 and 4 (below), a proportional and positive correlation was found among the observed antibody titers of coronavirus and the observed titers to other non-coronavirus pathogens. Table 3 includes ratios of geometric means (95% CI) from data comparing neutralizing antibody titers for various viral pathogens from commercial IVIG (left number within parentheses) and plasma from subjects administered ASCENIV (right number within parentheses). These data indicated that plasma samples identified as having elevated coronavirus titer value also possessed elevated titers to other non-coronavirus pathogens, such as RSV. Calculated fold changes in antibody titers are provided in Table 4, and demonstrate a 5.9 fold increase in coronavirus OC43 and a 5.5 fold increase in coronavirus 229E based on administration to a human subject of at least a 500 mg/kg dose of ASCENIV.

TABLE 3
Correlation Between Titers to RSV and Titers
to Non-RSV Respiratory Virus.

| | Ratio of geometric means | |
|---|---|---|
| | (95% CI) (ASCENIV/ | |
| Virus | commercial IGIV)^{a} | p Value^{b} |
| RSV | 1.861 (1.249, 2.771) | 0.003 |
| PIV1 | 1.792 (1.282, 2.505) | 0.001 |
| OC43 | 1.610 (1.127, 2.301) | 0.010 |
| PIV2 | 1.601 (1.160, 2.210) | 0.005 |
| 229E | 1.494 (1.144, 1.950) | 0.004 |
| Flu A | 1.402 (1.067, 1.843) | 0.016 |
| Flu B | 1.316 (1.026, 1.688) | 0.031 |
| hMPV | 1.264 (0.990, 1.613) | 0.060 |
| PIV 1 and 2 | 1.694 (1.250, 2.296) | 0.001 |
| OC43 and 229E | 1.551 (1.237, 1.945) | <0.001 |
| All viruse^{sc} | 1.529 (1.227, 1.907) | <0.001 |

^{a}Three randomly selected RI-002 batches and seven unselected commercial lots of IGIV from four different manufactures/brands
^{b}Two-group t-test for null hypothesis of no difference between the groups in geometric means (i.e., ratio of geometric means = 1).
^{c}Pooled RSV, respiratory syncytial virus; Flu A, influenza A; FluB, influenza B; hMPV, human metapneumovirus; PIV1, parainfluenza virus serotypes 1; PIV2, parainfluenza virus serotypes 2; OC43, coronavirus CoV OC43; 229E, coronaviruses CoV 229E.

TABLE 4
Calculated fold change in antibody titers
after ASCENIV administration.

| | Calculated Fold Change |
|---|---|
| Virus | Antibody Titers |
| RSV | 6.790 |
| PIV1 and 3 | 6.538 |
| OC43 | 5.874 |
| PIV2 | 5.841 |
| 229E | 5.451 |
| Flu A | 5.115 |
| Flu B | 4.802 |
| hMPV | 4.612 |
| PIV 1 and 2 | 6.181 |
| OC43 and 229E | 5.659 |
| All viruses^{a} | NA |

^{a}Pooled RSV, respiratory syncytial virus; Flu A, influenza A; FluB, influenza B; hMPV, human metapneumovirus; PIV1, parainfluenza virus serotypes 1; PIV2, parainfluenza virus serotypes 2; OC43, coronavirus CoV OC43; 229E, coronaviruses CoV 229E.

The above data is based on measured values for RSV neutralizing antibodies in ASCENIV at ≥500 mg/kg in phase 3 manuscript at fold increase of 6.790 and ratio of geometric means. Calculation: Fold change RSV/Ratio of means RSV=Fold change virus/Ratio of means virus. Calculated example for PIV1 and 3: 6.790/1.861=X/1.792; X=6.538 for PIV 1 and 3.

Together these data demonstrate that hyperimmune globulin compositions comprising pooled plasma samples and/or immunoglobulin prepared therefrom having increased neutralizing antibody titers against RSV, for example, also have elevated neutralizing antibody titers against coronavirus (coronavirus OC43, coronavirus 229E and if measured would have elevated titers to other respiratory viruses including coronavirus NL63, coronavirus HKU1, MERS-CoV, SARS-CoV, SARS-CoV-2 (COVID-19)). The data also suggest that hyperimmune globulin compositions comprising pooled plasma samples and/or immunoglobulin prepared therefrom having increased neutralizing antibody titers against coronavirus (coronavirus OC43, coronavirus 229E, coronavirus NL63, coronavirus HKU1, MERS-CoV, SARS-CoV, SARS-CoV-2 (COVID-19)) also have elevated neutralizing antibody titers against at least a second virus (e.g., RSV).

As described further herein, the compositions include pooled plasma samples and/or immunoglobulin prepared therefrom, which can be obtained from a plurality of donor human subjects (e.g., 100, 200, 300, 400, 500 or more subjects). In some embodiments, a pooled sample comprising higher neutralizing antibody titers against one virus can also have proportionally higher neutralizing antibody titers against other viruses. For example, as described further herein, pooled plasma samples can be obtained from a plurality of donor human subjects having increased antibody titers against a coronavirus (e.g., at least 1.2 fold greater than antibody titers from a corresponding control sample or an antibody neutralization titer from at least 40 to about 30,000), and these pooled plasma samples can also have proportionally increased antibody titers against at least a second virus (e.g., at least 1.1 fold greater than antibody titers from a corresponding control sample or an antibody neutralization titer from at least 40 to about 30,000), including, but not limited to, respiratory syncytial virus (RSV), influenza A virus, influenza B virus, parainfluenza virus type 1, parainfluenza virus type 2, metapneumovirus, coronavirus OC43, coronavirus 229E, coronavirus NL63, coronavirus HKU1, MERS-CoV, SARS-CoV, and SARS-CoV-2 (COVID-19).

Accordingly, the present disclosure provides a blending/pooling process that provides a pooled plasma composition or immunoglobulin prepared from same that contains a standardized and reproducible level of respiratory pathogen (e.g., coronavirus) specific antibodies thereby providing a heretofore unavailable, consistent and reproducible immunoglobulin product (e.g., for use as IVIG). Experiments confirmed that a pooled plasma composition or immunoglobulin prepared from the same (e.g., 2500 liters of pooled plasma from 1000 donors with a final RSV neutralization titer of 1800) could be consistently generated from different groups of 1000 donors. Further experiments confirmed that a pooled plasma composition (e.g., 2500 liters of pooled plasma from 1000 donors with a final RSV neutralization titer of 1800) contained antibody levels to tetanus, measles and polio that prevent, or protect from, infection with same, and also contained elevated antibody titer(s) specific for the respiratory pathogens described herein.

Materials and Methods

Enzyme immunoassay (EIA) was performed to detect virus-specific serum IgG for nine respiratory viruses: influenza A and B, RSV, parainfluenza (PIV) virus serotypes 1, 2 and 3, human metapneumovirus (hMPV), and coronavirus 229E (CoV 229E) and coronavirus OC43 per published methods (See, e.g., Falsey et al., J Am Geriatr Soc. 1992; 40:115-119; Falsey et al., J Am Geriatr Soc. 1995; 43:30-36; Falsey et al., J Am Geriatr Soc. 1997; 45:706-711; Falsey et al., J Infect Dis. 2003; 187:785-790). Briefly, antigens were produced from virally infected whole cell lysates for all viruses except RSV. Purified viral surface glycoproteins were used as antigen for RSV EIA according to published methods (See, e.g., Falsey et al., J Am Geriatr Soc. 1992; 40:115-119). Serial two-fold dilutions of each sample were tested in duplicate. Data analysis was performed via a paired-data approach. Data pairs were created by matching the donor ID within each ELISA assay run.

ELISA testing of IVIG was performed blinded to the type of sample. All samples were diluted with sample dilution buffer (PBS with 0.3% Tween 20 and 0.1 M EDTA) to a standard concentration of 50 mg of IgG per ml. Each viral antigen was diluted at previously determined concentration in bicarbonate buffer and coated separately on enzyme immunoassay microtiter plates and stored overnight in humidified chambers at 4° C. The following day, plates were washed and eight serial 2-fold dilutions in duplicate of unknown product were incubated on the antigen plates at room temperature in humidified chambers for 3 hours. The initial dilution of IVIG solution placed on antigen plates was 1:1600. Plates were then washed and bound IgG was detected with alkaline phosphatase conjugated goat anti-human IgG followed by substrate. A standard serum was included on each plate and the IgG titer for a specific virus was defined as the highest dilution with an optical density (OD) of 0.20.

Statistical Analysis. Titer data was tabulated with descriptive statistics of N (sample size, mean, geometric mean, standard deviation, minimum, median, and maximum). Difference between the RSV-IVIG and commercial IVIG (that is, Group 1 vs Group 2) were presented as the ratio of geometric means (RGM) and 95% Confidence intervals for the RGM was also provided. P-value for testing of the null hypothesis that the RGM equaled to 1 was produced based on 2-sample t-test at significance level of 0.05.

The properties of the IVIG from 1000 or more samples containing elevated levels of neutralizing antibody titers to one or more respiratory pathogens generated using the compositions and methods of the present disclosure is a significant advancement and improvement over other IVIG available in the art. In particular, the IVIG compositions of the present disclosure do not display or possess a neutralizing antibody titer for only a single pathogen (e.g., dominance for only one type of respiratory pathogens), but rather, through the methods of identifying donors and the blending processes developed and described herein, IVIG is provided that contains significantly elevated neutralizing titers to a plurality of respiratory pathogens and other pathogens, compared to the titers in 1000 randomly mixed plasma samples. The discovery of the use of neutralizing antibody titer to RSV (or other respiratory pathogen) as a biomarker to identify plasma donors that are high-titer selected donors (high/strong responders in general to respiratory pathogens (e.g., influenza A virus, influenza B virus, parainfluenza virus type 1, parainfluenza virus type 2, metapneumovirus, and coronavirus) makes possible the ability to identify donors and plasma that can be blended with non-high titer selected donors and non-selected donor plasma to provide a beneficial pooled plasma product. Thus, while an understanding of a mechanism is not needed to practice the embodiments of the present disclosure, and while these embodiments are not limited to any particular mechanism of action, in some embodiments, the present disclosure provides a heretofore unavailable pooled plasma composition (e.g., prepared according to the above described methods)) that contains a significant amount (e.g., greater than 50%) of non-high titer selected donor plasma (non-high titer RSV plasma) that provide therapeutic benefit not achievable with standard hyperimmune immune globulin (e.g., prepared from a limited number (e.g., 100-300) of plasma donors). In a further embodiment, due to the elevated levels of neutralizing antibody titers to one or a plurality of RSV, influenza A virus, influenza B virus, parainfluenza virus type 1, parainfluenza virus type 2, metapneumovirus, and coronavirus, such pooled plasma compositions provide a significantly improved therapeutic benefit to a subject administered the composition. For example, a pooled composition of the present disclosure, compared to pooled plasma samples obtained from 1000 or more random human subjects, provides viral neutralization properties against one or a plurality of respiratory pathogens or other pathogens that is not provided for by randomly pooled samples. In this way, a subject administered a composition of the present disclosure is able to fight off, or be treated for, infections that are not treatable with a composition of pooled plasma samples obtained from 1000 or more random human subjects or that are not treatable with a conventional hyperimmune immune globulin. For example, a pooled plasma composition according to the present disclosure (e.g., from 1000 or more samples wherein the pooled plasma composition comprises a neutralizing RSV antibody titer of 1800 or above and elevated levels of antibodies to one or more respiratory pathogens) when administered to a subject provides the subject the ability to fight off, or be treated for, infections that are not treatable with a composition of pooled plasma samples obtained from 1000 or more random human subjects and/or that are not treatable with a conventional hyperimmune immune globulin prepared from limited numbers of donors.

Whole Blood Assay. Automated whole blood analysis was carried out on blood samples collected in EDTA-containing tubes. Total number of white blood cells and lymphocytes was analyzed.

RSV plaque assay. Lung homogenates were clarified by centrifugation and diluted 1:10 and 1:100 in EMEM. Confluent HEp-2 monolayers in 24-well plates were infected in duplicates with 50 μl of sample per well starting with undiluted (neat) samples followed by diluted homogenates. After one hour incubation at 37° C. in a 5% CO₂ incubator, wells were overlayed with 0.75% methylcellulose medium and plates restored into the 37° C. incubator. After 4 days of incubation the overlay was removed and the cells were fixed with 0.1% crystal violet stain for one hour, then rinsed and air-dried. Plaques were counted and viral titers were expressed as plaque forming units per gram of tissue. Viral titer for a group was calculated as the geometric mean+standard error for all animals in that group at a given time. Student-t test was applied to determine significance of change in viral replication between vehicle-treated and test groups, with p<0.05 indicating a statistically-significant difference.

Real-time PCR. Total RNA was extracted from homogenized lung, kidney or liver tissue using the RNeasy purification kit (QIAGEN). One μg of total RNA was used to prepare cDNA using QuantiTect Reverse Transcription Kit (Qiagen). For the real-time PCR reactions the QuantiFast SYBR Green PCR Kit (Qiagen) was used in a final volume of 25 μl, with final primer concentrations of 0.5 μM. Reactions were set up in 96-well trays. Amplifications were performed on a Bio-Rad iCycler for 1 cycle of 95° C. for 3 min, followed by 40 cycles of 95° C. for 10 sec, 60° C. for 10 sec, and 72° C. for 15 sec. The baseline cycles and cycle threshold (Ct) were calculated by the iQ5 software in the PCR Base Line Subtracted

Curve Fit mode. Relative quantification of DNA was applied to all samples. The standard curves were developed using serially-diluted cDNA sample most enriched in the transcript of interest (e.g., lungs from day 4 post-primary RSV infection). The Ct values were plotted against log₁₀ cDNA dilution factor. These curves were used to convert the Ct values obtained for different samples to relative expression units. These relative expression units were then normalized to the level of β-actin mRNA (“housekeeping gene”) expressed in the corresponding sample. For animal studies, mRNA levels were expressed as the geometric mean±SEM for all animals in a group at a given time.

Case Study: Clinical Use of a Hyperimmune Globulin in an Adult with Severe Acute Respiratory Distress Syndrome and Confirmed COVID-19 Disease

As demonstrated by the following clinical case study, the compositions and methods of the present disclosure are effective for treating a coronavirus infection in a human subject.

A 70-year-old African American male with bronchiectasis presented to the emergency room with a one week history of increasing dyspnea, dry cough, sudden onset of high-grade fevers and body chills. The patient's condition progressed to severe respiratory compromise and he was admitted to the hospital's intensive care unit (ICU). Initial blood chemistry was unremarkable. A complete blood count revealed a mild shift to the left and lymphopenia. Pan cultures were drawn and a complete viral panel was ordered including a COVID-19 diagnostic, which was later confirmed positive. Patient was empirically initiated on broad-spectrum anti-infectives, and methylprednisolone

On day 3 post-admission, saturating oxygen (502c) was 94% with pO₂ 66 mmHg. A CT scan of the chest indicated progression of pulmonary disease with diffuse, primarily peripheral ground-glass opacities, consistent with COVID-19 pneumonia and atelectatic change in the left lower lobe with traction bronchiectasis. The patient was placed on high-flow nasal cannula (HFNC).

One week after admission, the patient's respiratory status further deteriorated with PaO₂ of 48 mmHg. A PaO₂/fraction of inspired oxygen (FiO₂; P/F) ratio of 80 was consistent with rapid progression to severe acute respiratory distress syndrome. The patient was intubated and placed on mechanical ventilation. Subsequent CT scans indicated bilateral pulmonary diffuse interstitial and ground-glass opacities with new trace left pleural effusion demonstrating progression of disease. Interleukin-6 serum levels were elevated at 29.3 pg/ml (reference: 0.0-15.5 pg/mL), prompting initiation of tocilizumab. Despite ongoing management and therapeutic interventions, respiratory function further declined during the next 7 days, as evidenced by increased density of ground glass opacities per CT scan and increasing respiratory distress.

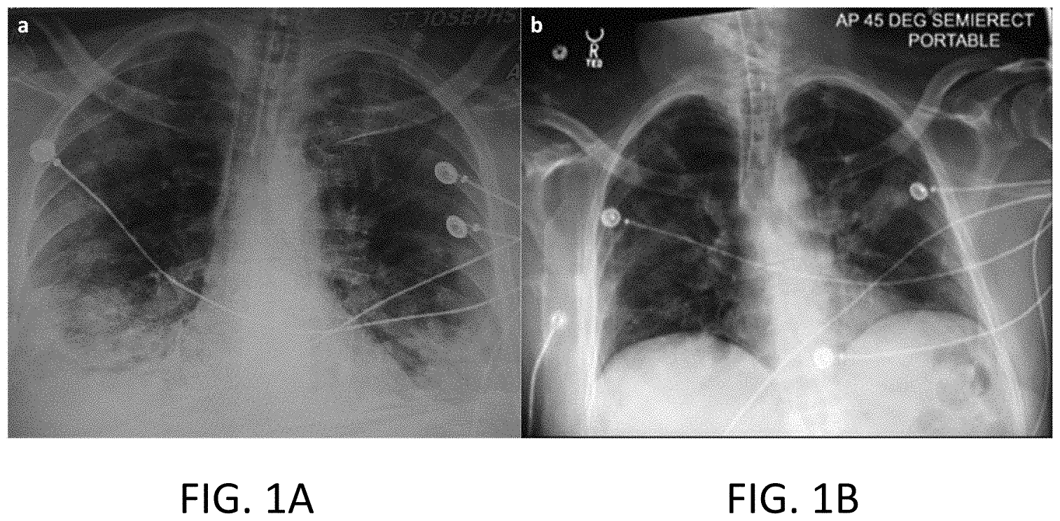

On Day 11, patient was administered a novel immune globulin intravenous (IGIV), human-slra, 10% (ADMA Biologics) liquid therapeutic at 1500 mg/kg. Several broad-spectrum anti-infectives were discontinued and patient continued on hydroxychloroquine. Pulmonary status further declined over the course of several days, with continued progression of pneumonia (FIG. 1a). Worsening aeration of the lungs was noted with an associated continuation on mechanical ventilation and a P/F ratio of 200. Patient developed a new fever spike, prompting anti-infective modification and initiation of meropenem and micafungin. The patient received a second dose of the same IGIV, human-slra, 10% liquid on day 14 at 750 mg/kg. Tracheal aspirate swab continued to be negative for the respiratory viral pathogen panel. Four days later post Ig infusion, respiratory function improved with a positive end-expiratory pressure of 6 at 40% FiO₂. The patient was successfully extubated and placed on BIPAP followed by HFNC. Patient remained positive for SARS-CoV-2 infection (FIG. 1b). Day 28, 502c was 99% with an improved P/F ratio of 371. Patient showed no signs of dyspnea, fever, and chills, and was transferred to the rehabilitation unit after confirming negative for SARS-CoV-2. Patient was discharged from the hospital on Day 34.

This case describes the use of a polyclonal hyperimmune globulin composition containing elevated levels of antibodies to multiple respiratory viruses to treat a COVID-positive patient with rapidly progressive respiratory disease who failed multiple medical interventions. Previous research has demonstrated the clinical benefits of polyclonal immune globulin and hyperimmune globulin for treatment of many viral-mediated infection such as polio, measles, mumps, influenza A and B, H1N1, Ebola and coronaviruses including MERS-CoV, severe acute respiratory [SAR] virus, and SARS-CoV-2. However, in this specific case report, the specific Ig administered was unique in its plasma pool composition to standardize antibody concentrations to meet specifications for polio, measles, diphtheria and RSV. Although the anti-infective interventions administered to the patient may have synergized with the infused uniquely formulated Ig composition, it is worthwhile to note that the patient's improved pulmonary distress was not evident until after the administration of the novel immune globulin intravenous (IGIV), human-slra, 10% (ADMA Biologics) liquid therapeutic composition. Despite being administered late in the course of the COVID-19 disease, the high loading dose of this unique Ig composition containing elevated levels of anti-viral antibodies coupled with its anti-inflammatory effects demonstrates its clinical efficacy in treating viral infections.

Various modification, recombination, and variation of the described features and embodiments will be apparent to those skilled in the art without departing from the scope and spirit of embodiments of the present disclosure. Although specific embodiments have been described, it should be understood that the embodiment of the present disclosure as claimed should not be unduly limited to such specific embodiments. Indeed, various modifications of the described modes and embodiments that are obvious to those skilled in the relevant fields are intended to be within the scope of the following claims. All publications and patents mentioned in the present application and/or listed below are herein incorporated by reference in their entireties.

Sequences

Sequences referenced in the present disclosure are provided below:
SARS-CoV-2 (COVID-19); NCBI GenBank accession code MN908947:
MESLVPGFNEKTHVQLSLPVLQVRDVLVRGFGDSVEEVLSEARQHLKDGTCGLVEVEK
GVLPQLEQPYVFIKRSDARTAPHGHVMVELVAELEGIQYGRSGETLGVLVPHVGEIPVA
YRKVLLRKNGNKGAGGHSYGADLKSFDLGDELGTDPYEDFQENWNTKHSSGVTRELM
RELNGGAYTRYVDNNFCGPDGYPLECIKDLLARAGKASCTLSEQLDFIDTKRGVYCCRE
HEHEIAWYTERSEKSYELQTPFEIKLAKKFDTFNGECPNFVFPLNSIIKTIQPRVEKKKLD
GFMGRIRSVYPVASPNECNQMCLSTLMKCDHCGETSWQTGDFVKATCEFCGTENLTKE
GATTCGYLPQNAVVKIYCPACHNSEVGPEHSLAEYHNESGLKTILRKGGRTIAFGGCVF
SYVGCHNKCAYWVPRASANIGCNHTGVVGEGSEGLNDNLLEILQKEKVNINIVGDFKL
NEEIAIILASFSASTSAFVETVKGLDYKAFKQIVESCGNFKVTKGKAKKGAWNIGEQKSIL
SPLYAFASEAARVVRSIFSRTLETAQNSVRVLQKAAITILDGISQYSLRLIDAMMFTSDLA
TNNLVVMAYITGGVVQLTSQWLTNIFGTVYEKLKPVLDWLEEKFKEGVEFLRDGWEIV
KFISTCACEIVGGQIVTCAKEIKESVQTFFKLVNKFLALCADSIIIGGAKLKALNLGETFVT
HSKGLYRKCVKSREETGLLMPLKAPKEIIFLEGETLPTEVLTEEVVLKTGDLQPLEQPTSE
AVEAPLVGTPVCINGLMLLEIKDTEKYCALAPNMMVTNNTFTLKGGAPTKVTFGDDTVI
EVQGYKSVNITFELDERIDKVLNEKCSAYTVELGTEVNEFACVVADAVIKTLQPVSELLT
PLGIDLDEWSMATYYLFDESGEFKLASHMYCSFYPPDEDEEEGDCEEEEFEPSTQYEYG
TEDDYQGKPLEFGATSAALQPEEEQEEDWLDDDSQQTVGQQDGSEDNQTTTIQTIVEVQ
PQLEMELTPVVQTIEVNSFSGYLKLTDNVYIKNADIVEEAKKVKPTVVVNAANVYLKH
GGGVAGALNKATNNAMQVESDDYIATNGPLKVGGSCVLSGHNLAKHCLHVVGPNVN
KGEDIQLLKSAYENFNQHEVLLAPLLSAGIFGADPIHSLRVCVDTVRTNVYLAVFDKNL
YDKLVSSFLEMKSEKQVEQKIAEIPKEEVKPFITESKPSVEQRKQDDKKIKACVEEVTTTL
EETKFLTENLLLYIDINGNLHPDSATLVSDIDITFLKKDAPYIVGDVVQEGVLTAVVIPTK
KAGGTTEMLAKALRKVPTDNYITTYPGQGLNGYTVEEAKTVLKKCKSAFYILPSIISNEK
QEILGTVSWNLREMLAHAEETRKLMPVCVETKAIVSTIQRKYKGIKIQEGVVDYGARFY
FYTSKTTVASLINTLNDLNETLVTMPLGYVTHGLNLEEAARYMRSLKVPATVSVSSPDA
VTAYNGYLTSSSKTPEEHFIETISLAGSYKDWSYSGQSTQLGIEFLKRGDKSVYYTSNPTT
FHLDGEVITFDNLKTLLSLREVRTIKVFTTVDNINLHTQVVDMSMTYGQQFGPTYLDGA
DVTKIKPHNSHEGKTFYVLPNDDTLRVEAFEYYHTTDPSFLGRYMSALNHTKKWKYPQ
VNGLTSIKWADNNCYLATALLTLQQIELKFNPPALQDAYYRARAGEAANFCALILAYCN
KTVGELGDVRETMSYLFQHANLDSCKRVLNVVCKTCGQQQTTLKGVEAVMYMGTLS
YEQFKKGVQIPCTCGKQATKYLVQQESPFVMMSAPPAQYELKHGTFTCASEYTGNYQC
GHYKHITSKETLYCIDGALLTKSSEYKGPITDVFYKENSYTTTIKPVTYKLDGVVCTEIDP
KLDNYYKKDNSYFTEQPIDLVPNQPYPNASFDNFKFVCDNIKFADDLNQLTGYKKPASR
ELKVTFFPDLNGDVVAIDYKHYTPSFKKGAKLLHKPIVWHVNNATNKATYKPNTWCIR
CLWSTKPVETSNSFDVLKSEDAQGMDNLACEDLKPVSEEVVENPTIQKDVLECNVKTTE
VVGDIILKPANNSLKITEEVGHTDLMAAYVDNSSLTIKKPNELSRVLGLKTLATHGLAAV
NSVPWDTIANYAKPFLNKVVSTTTNIVTRCLNRVCTNYMPYFFTLLLQLCTFTRSTNSRI
KASMPTTIAKNTVKSVGKFCLEASFNYLKSPNFSKLINIIIWFLLLSVCLGSLIYSTAALGV
LMSNLGMPSYCTGYREGYLNSTNVTIATYCTGSIPCSVCLSGLDSLDTYPSLETIQITISSF
KWDLTAFGLVAEWFLAYILFTRFFYVLGLAAIMQLFFSYFAVHFISNSWLMWLIINLVQ
MAPISAMVRMYIFFASFYYVWKSYVHVVDGCNSSTCMMCYKRNRATRVECTTIVNGV
RRSFYVYANGGKGFCKLHNWNCVNCDTFCAGSTFISDEVARDLSLQFKRPINPTDQSSY
IVDSVTVKNGSIHLYFDKAGQKTYERHSLSHFVNLDNLRANNTKGSLPINVIVFDGKSKC
EESSAKSASVYYSQLMCQPILLLDQALVSDVGDSAEVAVKMFDAYVNTFSSTFNVPME
KLKTLVATAEAELAKNVSLDNVLSTFISAARQGFVDSDVETKDVVECLKLSHQSDIEVT
GDSCNNYMLTYNKVENMTPRDLGACIDCSARHINAQVAKSHNIALIWNVKDFMSLSEQ
LRKQIRSAAKKNNLPFKLTCATTRQVVNVVTTKIALKGGKIVNNWLKQLIKVTLVFLFV
AAIFYLITPVHVMSKHTDFSSEIIGYKAIDGGVTRDIASTDTCFANKHADFDTWFSQRGG
SYTNDKACPLIAAVITREVGFVVPGLPGTILRTTNGDFLHFLPRVFSAVGNICYTPSKLIE
YTDFATSACVLAAECTIFKDASGKPVPYCYDTNVLEGSVAYESLRPDTRYVLMDGSIIQF
PNTYLEGSVRVVTTFDSEYCRHGTCERSEAGVCVSTSGRWVLNNDYYRSLPGVFCGVD
AVNLLTNMFTPLIQPIGALDISASIVAGGIVAIVVTCLAYYFMRFRRAFGEYSHVVAFNTL
LFLMSFTVLCLTPVYSFLPGVYSVIYLYLTFYLTNDVSFLAHIQWMVMFTPLVPFWITIA
YIICISTKHFYWFFSNYLKRRVVFNGVSFSTFEEAALCTFLLNKEMYLKLRSDVLLPLTQ
YNRYLALYNKYKYFSGAMDTTSYREAACCHLAKALNDFSNSGSDVLYQPPQTSITSAV
LQSGFRKMAFPSGKVEGCMVQVTCGTTTLNGLWLDDVVYCPRHVICTSEDMLNPNYE
DLLIRKSNHNFLVQAGNVQLRVIGHSMQNCVLKLKVDTANPKTPKYKFVRIQPGQTFSV
LACYNGSPSGVYQCAMRPNFTIKGSFLNGSCGSVGFNIDYDCVSFCYMHHMELPTGVH
AGTDLEGNFYGPFVDRQTAQAAGTDTTITVNVLAWLYAAVINGDRWFLNRFTTTLNDF
NLVAMKYNYEPLTQDHVDILGPLSAQTGIAVLDMCASLKELLQNGMNGRTILGSALLE
DEFTPFDVVRQCSGVTFQSAVKRTIKGTHHWLLLTILTSLLVLVQSTQWSLFFFLYENAF
LPFAMGIIAMSAFAMMFVKHKHAFLCLFLLPSLATVAYFNMVYMPASWVMRIMTWLD
MVDTSLSGFKLKDCVMYASAVVLLILMTARTVYDDGARRVWTLMNVLTLVYKVYYG
NALDQAISMWALIISVTSNYSGVVTTVMFLARGIVFMCVEYCPIFFITGNTLQCIMLVYC
FLGYFCTCYFGLFCLLNRYFRLTLGVYDYLVSTQEFRYMNSQGLLPPKNSIDAFKLNIKL
LGVGGKPCIKVATVQSKMSDVKCTSVVLLSVLQQLRVESSSKLWAQCVQLHNDILLAK
DTTEAFEKMVSLLSVLLSMQGAVDINKLCEEMLDNRATLQAIASEFSSLPSYAAFATAQ
EAYEQAVANGDSEVVLKKLKKSLNVAKSEFDRDAAMQRKLEKMADQAMTQMYKQA
RSEDKRAKVTSAMQTMLFTMLRKLDNDALNNIINNARDGCVPLNIIPLTTAAKLMVVIP
DYNTYKNTCDGTTFTYASALWEIQQVVDADSKIVQLSEISMDNSPNLAWPLIVTALRAN
SAVKLQNNELSPVALRQMSCAAGTTQTACTDDNALAYYNTTKGGRFVLALLSDLQDL
KWARFPKSDGTGTIYTELEPPCRFVTDTPKGPKVKYLYFIKGLNNLNRGMVLGSLAATV
RLQAGNATEVPANSTVLSFCAFAVDAAKAYKDYLASGGQPITNCVKMLCTHTGTGQAI
TVTPEANMDQESFGGASCCLYCRCHIDHPNPKGFCDLKGKYVQIPTTCANDPVGFTLKN
TVCTVCGMWKGYGCSCDQLREPMLQSADAQSFLNRVCGVSAARLTPCGTGTSTDVVY
RAFDIYNDKVAGFAKFLKTNCCRFQEKDEDDNLIDSYFVVKRHTFSNYQHEETIYNLLK
DCPAVAKHDFFKFRIDGDMVPHISRQRLTKYTMADLVYALRHFDEGNCDTLKEILVTY
NCCDDDYFNKKDWYDFVENPDILRVYANLGERVRQALLKTVQFCDAMRNAGIVGVLT
LDNQDLNGNWYDFGDFIQTTPGSGVPVVDSYYSLLMPILTLTRALTAESHVDTDLTKPY
IKWDLLKYDFTEERLKLFDRYFKYWDQTYHPNCVNCLDDRCILHCANFNVLFSTVFPPT
SFGPLVRKIFVDGVPFVVSTGYHFRELGVVHNQDVNLHSSRLSFKELLVYAADPAMHA
ASGNLLLDKRTTCFSVAALTNNVAFQTVKPGNFNKDFYDFAVSKGFFKEGSSVELKHFF
FAQDGNAAISDYDYYRYNLPTMCDIRQLLFVVEVVDKYFDCYDGGCINANQVIVNNLD
KSAGFPFNKWGKARLYYDSMSYEDQDALFAYTKRNVIPTITQMNLKYAISAKNRARTV
AGVSICSTMTNRQFHQKLLKSIAATRGATVVIGTSKFYGGWHNMLKTVYSDVENPHLM
GWDYPKCDRAMPNMLRIMASLVLARKHTTCCSLSHRFYRLANECAQVLSEMVMCGGS
LYVKPGGTSSGDATTAYANSVFNICQAVTANVNALLSTDGNKIADKYVRNLQHRLYEC
LYRNRDVDTDFVNEFYAYLRKHFSMMILSDDAVVCFNSTYASQGLVASIKNFKSVLYY
QNNVFMSEAKCWTETDLTKGPHEFCSQHTMLVKQGDDYVYLPYPDPSRILGAGCFVDD
IVKTDGTLMIERFVSLAIDAYPLTKHPNQEYADVFHLYLQYIRKLHDELTGHMLDMYSV
MLTNDNTSRYWEPEFYEAMYTPHTVLQAVGACVLCNSQTSLRCGACIRRPFLCCKCCY
DHVISTSHKLVLSVNPYVCNAPGCDVTDVTQLYLGGMSYYCKSHKPPISFPLCANGQVF
GLYKNTCVGSDNVTDFNAIATCDWTNAGDYILANTCTERLKLFAAETLKATEETFKLSY
GIATVREVLSDRELHLSWEVGKPRPPLNRNYVFTGYRVTKNSKVQIGEYTFEKGDYGDA
VVYRGTTTYKLNVGDYFVLTSHTVMPLSAPTLVPQEHYVRITGLYPTLNISDEFSSNVA
NYQKVGMQKYSTLQGPPGTGKSHFAIGLALYYPSARIVYTACSHAAVDALCEKALKYL
PIDKCSRIIPARARVECFDKFKVNSTLEQYVFCTVNALPETTADIVVFDEISMATNYDLSV
VNARLRAKHYVYIGDPAQLPAPRTLLTKGTLEPEYFNSVCRLMKTIGPDMFLGTCRRCP
AEIVDTVSALVYDNKLKAHKDKSAQCFKMFYKGVITHDVSSAINRPQIGVVREFLTRNP
AWRKAVFISPYNSQNAVASKILGLPTQTVDSSQGSEYDYVIFTQTTETAHSCNVNRFNV
AITRAKVGILCIMSDRDLYDKLQFTSLEIPRRNVATLQAENVTGLFKDCSKVITGLHPTQ
APTHLSVDTKFKTEGLCVDIPGIPKDMTYRRLISMMGFKMNYQVNGYPNMFITREEAIR
HVRAWIGFDVEGCHATREAVGTNLPLQLGFSTGVNLVAVPTGYVDTPNNTDFSRVSAK
PPPGDQFKHLIPLMYKGLPWNVVRIKIVQMLSDTLKNLSDRVVFVLWAHGFELTSMKY
FVKIGPERTCCLCDRRATCFSTASDTYACWHHSIGFDYVYNPFMIDVQQWGFTGNLQSN
HDLYCQVHGNAHVASCDAIMTRCLAVHECFVKRVDWTIEYPIIGDELKINAACRKVQH
MVVKAALLADKFPVLHDIGNPKAIKCVPQADVEWKFYDAQPCSDKAYKIEELFYSYAT
HSDKFTDGVCLFWNCNVDRYPANSIVCRFDTRVLSNLNLPGCDGGSLYVNKHAFHTPA
FDKSAFVNLKQLPFFYYSDSPCESHGKQVVSDIDYVPLKSATCITRCNLGGAVCRHHAN
EYRLYLDAYNMMISAGFSLWVYKQFDTYNLWNTFTRLQSLENVAFNVVNKGHFDGQQ
GEVPVSIINNTVYTKVDGVDVELFENKTTLPVNVAFELWAKRNIKPVPEVKILNNLGVDI
AANTVIWDYKRDAPAHISTIGVCSMTDIAKKPTETICAPLTVFFDGRVDGQVDLFRNAR
NGVLITEGSVKGLQPSVGPKQASLNGVTLIGEAVKTQFNYYKKVDGVVQQLPETYFTQS
RNLQEFKPRSQMEIDFLELAMDEFIERYKLEGYAFEHIVYGDFSHSQLGGLHLLIGLAKR
FKESPFELEDFIPMDSTVKNYFITDAQTGSSKCVCSVIDLLLDDFVEIIKSQDLSVVSKVV
KVTIDYTEISFMLWCKDGHVETFYPKLQSSQAWQPGVAMPNLYKMQRMLLEKCDLQN
YGDSATLPKGIMMNVAKYTQLCQYLNTLTLAVPYNMIRVIHFGAGSDKGVAPGTAVLR
QWLPTGTLLVDSDLNDFVSDADSTLIGDCATVHTANKWDLIISDMYDPKTKNVTKEND
SKEGFFTYICGFIQQKLALGGSVAIKITEHSWNADLYKLMGHFAWWTAFVTNVNASSSE
AFLIGCNYLGKPREQIDGYVMHANYIFWRNTNPIQLSSYSLFDMSKFPLKLRGTAVMSL
KEGQINDMILSLLSKGRLIIRENNRVVISSDVLVNN (SEQ ID NO: 1)
SARS-CoV; NCBI GenBank accession code AY274119:
MESLVLGVNEKTHVQLSLPVLQVRDVLVRGFGDSVEEALSEAREHLKNGTCGLVELEK
GVLPQLEQPYVFIKRSDALSTNHGHKVVELVAEMDGIQYGRSGITLGVLVPHVGETPIA
YRNVLLRKNGNKGAGGHSYGIDLKSYDLGDELGTDPIEDYEQNWNTKHGSGALRELTR
ELNGGAVTRYVDNNFCGPDGYPLDCIKDFLARAGKSMCTLSEQLDYIESKRGVYCCRD
HEHEIAWFTERSDKSYEHQTPFEIKSAKKFDTFKGECPKFVFPLNSKVKVIQPRVEKKKT
EGFMGRIRSVYPVASPQECNNMHLSTLMKCNHCDEVSWQTCDFLKATCEHCGTENLVI
EGPTTCGYLPTNAVVKMPCPACQDPEIGPEHSVADYHNHSNIETRLRKGGRTRCFGGCV
FAYVGCYNKRAYWVPRASADIGSGHTGITGDNVETLNEDLLEILSRERVNINIVGDFHLN
EEVAIILASFSASTSAFIDTIKSLDYKSFKTIVESCGNYKVTKGKPVKGAWNIGQQRSVLT
PLCGFPSQAAGVIRSIFARTLDAANHSIPDLQRAAVTILDGISEQSLRLVDAMVYTSDLLT
NSVIIMAYVTGGLVQQTSQWLSNLLGTTVEKLRPIFEWIEAKLSAGVEFLKDAWEILKFL
ITGVFDIVKGQIQVASDNIKDCVKCFIDVVNKALEMCIDQVTIAGAKLRSLNLGEVFIAQS
KGLYRQCIRGKEQLQLLMPLKAPKEVTFLEGDSHDTVLTSEEVVLKNGELEALETPVDS
FTNGAIVGTPVCVNGLMLLEIKDKEQYCALSPGLLATNNVFRLKGGAPIKGVTFGEDTV
WEVQGYKNVRITFELDERVDKVLNEKCSVYTVESGTEVTEFACVVAEAVVKTLQPVSD
LLTNMGIDLDEWSVATFYLFDDAGEENFSSRMYCSFYPPDEEEEDDAECEEEEIDETCEH
EYGTEDDYQGLPLEFGASAETVRVEEEEEEDWLDDTTEQSEIEPEPEPTPEEPVNQFTGY
LKLTDNVAIKCVDIVKEAQSANPMVIVNAANIHLKHGGGVAGALNKATNGAMQKESD
DYIKLNGPLTVGGSCLLSGHNLAKKCLHVVGPNLNAGEDIQLLKAAYENFNSQDILLAP
LLSAGIFGAKPLQSLQVCVQTVRTQVYIAVNDKALYEQVVMDYLDNLKPRVEAPKQEE
PPNTEDSKTEEKSVVQKPVDVKPKIKACIDEVTTTLEETKFLTNKLLLFADINGKLYHDS
QNMLRGEDMSFLEKDAPYMVGDVITSGDITCVVIPSKKAGGTTEMLSRALKKVPVDEYI
TTYPGQGCAGYTLEEAKTALKKCKSAFYVLPSEAPNAKEEILGTVSWNLREMLAHAEE
TRKLMPICMDVRAIMATIQRKYKGIKIQEGIVDYGVRFFFYTSKEPVASIITKLNSLNEPL
VTMPIGYVTHGFNLEEAARCMRSLKAPAVVSVSSPDAVTTYNGYLTSSSKTSEEHFVET
VSLAGSYRDWSYSGQRTELGVEFLKRGDKIVYHTLESPVEFHLDGEVLSLDKLKSLLSL
REVKTIKVFTTVDNTNLHTQLVDMSMTYGQQFGPTYLDGADVTKIKPHVNHEGKTFFV
LPSDDTLRSEAFEYYHTLDESFLGRYMSALNHTKKWKFPQVGGLTSIKWADNNCYLSS
VLLALQQLEVKFNAPALQEAYYRARAGDAANFCALILAYSNKTVGELGDVRETMTHLL
QHANLESAKRVLNVVCKHCGQKTTTLTGVEAVMYMGTLSYDNLKTGVSIPCVCGRDA
TQYLVQQESSFVMMSAPPAEYKLQQGTFLCANEYTGNYQCGHYTHITAKETLYRIDGA
HLTKMSEYKGPVTDVFYKETSYTTTIKPVSYKLDGVTYTEIEPKLDGYYKKDNAYYTEQ
PIDLVPTQPLPNASFDNFKLTCSNTKFADDLNQMTGFTKPASRELSVTFFPDLNGDVVAI
DYRHYSASFKKGAKLLHKPIVWHINQATTKTTFKPNTWCLRCLWSTKPVDTSNSFEVL
AVEDTQGMDNLACESQQPTSEEVVENPTIQKEVIECDVKTTEVVGNVILKPSDEGVKVT
QELGHEDLMAAYVENTSITIKKPNELSLALGLKTIATHGIAAINSVPWSKILAYVKPFLGQ
AAITTSNCAKRLAQRVFNNYMPYVFTLLFQLCTFTKSTNSRIRASLPTTIAKNSVKSVAK
LCLDAGINYVKSPKFSKLFTIAMWLLLLSICLGSLICVTAAFGVLLSNFGAPSYCNGVREL
YLNSSNVTTMDFCEGSFPCSICLSGLDSLDSYPALETIQVTISSYKLDLTILGLAAEWVLA
YMLFTKFFYLLGLSAIMQVFFGYFASHFISNSWLMWFIISIVQMAPVSAMVRMYIFFASF
YYIWKSYVHIMDGCTSSTCMMCYKRNRATRVECTTIVNGMKRSFYVYANGGRGFCKT
HNWNCLNCDTFCTGSTFISDEVARDLSLQFKRPINPTDQSSYIVDSVAVKNGALHLYFD
KAGQKTYERHPLSHFVNLDNLRANNTKGSLPINVIVFDGKSKCDESASKSASVYYSQLM
CQPILLLDQALVSDVGDSTEVSVKMFDAYVDTFSATFSVPMEKLKALVATAHSELAKG
VALDGVLSTFVSAARQGVVDTDVDTKDVIECLKLSHHSDLEVTGDSCNNFMLTYNKVE
NMTPRDLGACIDCNARHINAQVAKSHNVSLIWNVKDYMSLSEQLRKQIRSAAKKNNIPF
RLTCATTRQVVNVITTKISLKGGKIVSTCFKLMLKATLLCVLAALVCYIVMPVHTLSIHD
GYTNEIIGYKAIQDGVTRDIISTDDCFANKHAGFDAWFSQRGGSYKNDKSCPVVAAIITR
EIGFIVPGLPGTVLRAINGDFLHFLPRVFSAVGNICYTPSKLIEYSDFATSACVLAAECTIF
KDAMGKPVPYCYDTNLLEGSISYSELRPDTRYVLMDGSIIQFPNTYLEGSVRVVTTFDAE
YCRHGTCERSEVGICLSTSGRWVLNNEHYRALSGVFCGVDAMNLIANIFTPLVQPVGAL
DVSASVVAGGIIAILVTCAAYYFMKFRRVFGEYNHVVAANALLFLMSFTILCLVPAYSFL
PGVYSVFYLYLTFYFTNDVSFLAHLQWFAMFSPIVPFWITAIYVFCISLKHCHWFFNNYL
RKRVMFNGVTFSTFEEAALCTFLLNKEMYLKLRSETLLPLTQYNRYLALYNKYKYFSG
ALDTTSYREAACCHLAKALNDFSNSGADVLYQPPQTSITSAVLQSGFRKMAFPSGKVEG
CMVQVTCGTTTLNGLWLDDTVYCPRHVICTAEDMLNPNYEDLLIRKSNHSFLVQAGNV
QLRVIGHSMQNCLLRLKVDTSNPKTPKYKFVRIQPGQTFSVLACYNGSPSGVYQCAMRP
NHTIKGSFLNGSCGSVGFNIDYDCVSFCYMHHMELPTGVHAGTDLEGKFYGPFVDRQT
AQAAGTDTTITLNVLAWLYAAVINGDRWFLNRFTTTLNDFNLVAMKYNYEPLTQDHV
DILGPLSAQTGIAVLDMCAALKELLQNGMNGRTILGSTILEDEFTPFDVVRQCSGVTFQG
KFKKIVKGTHHWMLLTFLTSLLILVQSTQWSLFFFVYENAFLPFTLGIMAIAACAMLLVK
HKHAFLCLFLLPSLATVAYFNMVYMPASWVMRIMTWLELADTSLSGYRLKDCVMYAS
ALVLLILMTARTVYDDAARRVWTLMNVITLVYKVYYGNALDQAISMWALVISVTSNYS
GVVTTIMFLARAIVFVCVEYYPLLFITGNTLQCIMLVYCFLGYCCCCYFGLFCLLNRYFR
LTLGVYDYLVSTQEFRYMNSQGLLPPKSSIDAFKLNIKLLGIGGKPCIKVATVQSKMSDV
KCTSVVLLSVLQQLRVESSSKLWAQCVQLHNDILLAKDTTEAFEKMVSLLSVLLSMQG
AVDINRLCEEMLDNRATLQAIASEFSSLPSYAAYATAQEAYEQAVANGDSEVVLKKLK
KSLNVAKSEFDRDAAMQRKLEKMADQAMTQMYKQARSEDKRAKVTSAMQTMLFTM
LRKLDNDALNNIINNARDGCVPLNIIPLTTAAKLMVVVPDYGTYKNTCDGNTFTYASAL
WEIQQVVDADSKIVQLSEINMDNSPNLAWPLIVTALRANSAVKLQNNELSPVALRQMSC
AAGTTQTACTDDNALAYYNNSKGGRFVLALLSDHQDLKWARFPKSDGTGTIYTELEPP
CRFVTDTPKGPKVKYLYFIKGLNNLNRGMVLGSLAATVRLQAGNATEVPANSTVLSFC
AFAVDPAKAYKDYLASGGQPITNCVKMLCTHTGTGQAITVTPEANMDQESFGGASCCL
YCRCHIDHPNPKGFCDLKGKYVQIPTTCANDPVGFTLRNTVCTVCGMWKGYGCSCDQL
REPLMQSADASTFFKRVCGVSAARLTPCGTGTSTDVVYRAFDIYNEKVAGFAKFLKTNC
CRFQEKDEEGNLLDSYFVVKRHTMSNYQHEETIYNLVKDCPAVAVHDFFKFRVDGDM
VPHISRQRLTKYTMADLVYALRHFDEGNCDTLKEILVTYNCCDDDYFNKKDWYDFVEN
PDILRVYANLGERVRQSLLKTVQFCDAMIRDAGIVGVLTLDNQDLNGNWYDFGDFVQV
APGCGVPIVDSYYSLLMPILTLTRALAAESHMDADLAKPLIKWDLLKYDFTEERLCLFD
RYFKYWDQTYHPNCINCLDDRCILHCANFNVLFSTVFPPTSFGPLVRKIFVDGVPFVVST
GYHFRELGVVHNQDVNLHSSRLSFKELLVYAADPAMHAASGNLLLDKRTTCFSVAALT
NNVAFQTVKPGNFNKDFYDFAVSKGFFKEGSSVELKHFFFAQDGNAAISDYDYYRYNL
PTMCDIRQLLFVVEVVDKYFDCYDGGCINANQVIVNNLDKSAGFPFNKWGKARLYYDS
MSYEDQDALFAYTKRNVIPTITQMNLKYAISAKNRARTVAGVSICSTMTNRQFHQKLLK
SIAATRGATVVIGTSKFYGGWHNMLKTVYSDVETPHLMGWDYPKCDRAMPNMLRIMA
SLVLARKHNTCCNLSHRFYRLANECAQVLSEMVMCGGSLYVKPGGTSSGDATTAYAN
SVFNICQAVTANVNALLSTDGNKIADKYVRNLQHRLYECLYRNRDVDHEFVDEFYAYL
RKHFSMMILSDDAVVCYNSNYAAQGLVASIKNFKAVLYYQNNVFMSEAKCWTETDLT
KGPHEFCSQHTMLVKQGDDYVYLPYPDPSRILGAGCFVDDIVKTDGTLMIERFVSLAID
AYPLTKHPNQEYADVFHLYLQYIRKLHDELTGHMLDMYSVMLTNDNTSRYWEPEFYE
AMYTPHTVLQAVGACVLCNSQTSLRCGACIRRPFLCCKCCYDHVISTSHKLVLSVNPYV
CNAPGCDVTDVTQLYLGGMSYYCKSHKPPISFPLCANGQVFGLYKNTCVGSDNVTDFN
AIATCDWTNAGDYILANTCTERLKLFAAETLKATEETFKLSYGIATVREVLSDRELHLS
WEVGKPRPPLNRNYVFTGYRVTKNSKVQIGEYTFEKGDYGDAVVYRGTTTYKLNVGD
YFVLTSHTVMPLSAPTLVPQEHYVRITGLYPTLNISDEFSSNVANYQKVGMQKYSTLQG
PPGTGKSHFAIGLALYYPSARIVYTACSHAAVDALCEKALKYLPIDKCSRIIPARARVECF
DKFKVNSTLEQYVFCTVNALPETTADIVVFDEISMATNYDLSVVNARLRAKHYVYIGDP
AQLPAPRTLLTKGTLEPEYFNSVCRLMKTIGPDMFLGTCRRCPAEIVDTVSALVYDNKL
KAHKDKSAQCFKMFYKGVITHDVSSAINRPQIGVVREFLTRNPAWRKAVFISPYNSQNA
VASKILGLPTQTVDSSQGSEYDYVIFTQTTETAHSCNVNRFNVAITRAKIGILCIMSDRDL
YDKLQFTSLEIPRRNVATLQAENVTGLFKDCSKIITGLHPTQAPTHLSVDIKFKTEGLCVD
IPGIPKDMTYRRLISMMGFKMNYQVNGYPNMFITREEAIRHVRAWIGFDVEGCHATRD
AVGTNLPLQLGFSTGVNLVAVPTGYVDTENNTEFTRVNAKPPPGDQFKHLIPLMYKGLP
WNVVRIKIVQMLSDTLKGLSDRVVFVLWAHGFELTSMKYFVKIGPERTCCLCDKRATC
FSTSSDTYACWNHSVGFDYVYNPFMIDVQQWGFTGNLQSNHDQHCQVHGNAHVASCD
AIMTRCLAVHECFVKRVDWSVEYPIIGDELRVNSACRKVQHMVVKSALLADKFPVLHD
IGNPKAIKCVPQAEVEWKFYDAQPCSDKAYKIEELFYSYATHHDKFTDGVCLFWNCNV
DRYPANAIVCRFDTRVLSNLNLPGCDGGSLYVNKHAFHTPAFDKSAFTNLKQLPFFYYS
DSPCESHGKQVVSDIDYVPLKSATCITRCNLGGAVCRHHANEYRQYLDAYNMMISAGF
SLWIYKQFDTYNLWNTFTRLQSLENVAYNVVNKGHFDGHAGEAPVSIINNAVYTKVDG
IDVEIFENKTTLPVNVAFELWAKRNIKPVPEIKILNNLGVDIAANTVIWDYKREAPAHVS
TIGVCTMTDIAKKPTESACSSLTVLFDGRVEGQVDLFRNARNGVLITEGSVKGLTPSKGP
AQASVNGVTLIGESVKTQFNYFKKVDGIIQQLPETYFTQSRDLEDFKPRSQMETDFLELA
MDEFIQRYKLEGYAFEHIVYGDFSHGQLGGLHLMIGLAKRSQDSPLKLEDFIPMDSTVK
NYFITDAQTGSSKCVCSVIDLLLDDFVEIIKSQDLSVISKVVKVTIDYAEISFMLWCKDGH
VETFYPKLQASQAWQPGVAMPNLYKMQRMLLEKCDLQNYGENAVIPKGIMMNVAKY
TQLCQYLNTLTLAVPYNMRVIHFGAGSDKGVAPGTAVLRQWLPTGTLLVDSDLNDFVS
DADSTLIGDCATVHTANKWDLIISDMYDPRTKHVTKENDSKEGFFTYLCGFIKQKLALG
GSIAVKITEHSWNADLYKLMGHFSWWTAFVTNVNASSSEAFLIGANYLGKPKEQIDGYT
MHANYIFWRNTNPIQLSSYSLFDMSKFPLKLRGTAVMSLKENQINDMIYSLLEKGRLIIR
ENNRVVVSSDILVNN (SEQ ID NO: 2)
MERS-CoV; NCBI GenBank accession code NC_019843:
MSFVAGVTAQGARGTYRAALNSEKHQDHVSLTVPLCGSGNLVEKLSPWFMDGENAYE
VVKAMLLKKEPLLYVPIRLAGHTRHLPGPRVYLVERLIACENPFMVNQLAYSSSANGSL
VGTTLQGKPIGMFFPYDIELVTGKQNILLRKYGRGGYHYTPFHYERDNTSCPEWMDDFE
ADPKGKYAQNLLKKLIGGDVTPVDQYMCGVDGKPISAYAFLMAKDGITKLADVEADV
AARADDEGFITLKNNLYRLVWHVERKDVPYPKQSIFTINSVVQKDGVENTPPHYFTLGC
KILTLTPRNKWSGVSDLSLKQKLLYTFYGKESLENPTYIYHSAFIECGSCGNDSWLTGNA
IQGFACGCGASYTANDVEVQSSGMIKPNALLCATCPFAKGDSCSSNCKHSVAQLVSYLS
ERCNVIADSKSFTLIFGGVAYAYFGCEEGTMYFVPRAKSVVSRIGDSIFTGCTGSWNKVT
QIANMFLEQTQHSLNFVGEFVVNDVVLAILSGTTTNVDKIRQLLKGVTLDKLRDYLADY
DVAVTAGPFMDNAINVGGTGLQYAAITAPYVVLTGLGESFKKVATIPYKVCNSVKDTL
AYYAHSVLYRVFPYDMDSGVSSFSELLFDCVDLSVASTYFLVRILQDKTGDFMSTIITSC
QTAVSKLLDTCFEATEATFNFLLDLAGLFRIFLRNAYVYTSQGFVVVNGKVSTLVKQVL
DLLNKGMQLLHTKVSWAGSKIIAVIYSGRESLIFPSGTYYCVTTKAKSVQQDLDVILPGE
FSKKQLGLLQPTDNSTTVSVTVSSNMVETVVGQLEQTNMHSPDVIVGDYVIISEKLFVRS
KEEDGFAFYPACTNGHAVPTLFRLKGGAPVKKVAFGGDQVHEVAAVRSVTVEYNIHA
VLDTLLASSSLRTFVVDKSLSIEEFADVVKEQVSDLLVKLLRGMPIPDFDLDDFIDAPCY
CFNAEGDASWSSTMIFSLHPVECDEECSEVEASDLEEGESECISETSTEQVDVSHETSDDE
WAAAVDEAFPLDEAEDVTESVQEEAQPVEVPVEDIAQVVIADTLQETPVVPDTVEVPPQ
VVKLPSAPQTIQPEVKEVAPVYEADTEQTQNVTVKPKRLRKKRNVDPLSNFEHKVITEC
VTIVLGDAIQVAKCYGESVLVNAANTHLKHGGGIAGAINAASKGAVQKESDEYILAKGP
LQVGDSVLLQGHSLAKNILHVVGPDARAKQDVSLLSKCYKAMNAYPLVVTPLVSAGIF
GVKPAVSFDYLIREAKTRVLVVVNSQDVYKSLTIVDIPQSLTFSYDGLRGAIRKAKDYGF
TVFVCTDNSANTKVLRNKGVDYTKKFLTVDGVQYYCYTSKDTLDDILQQANKSVGIIS
MPLGYVSHGLDLMQAGSVVRRVNVPYVCLLANKEQEAILMSEDVKLNPSEDFIKHVRT
NGGYNSWHLVEGELLVQDLRLNKLLHWSDQTICYKDSVFYVVKNSTAFPFETLSACRA
YLDSRTTQQLTIEVLVTVDGVNFRTVVLNNKNTYRSQLGCVFFNGADISDTIPDEKQNG
HSLYLADNLTADETKALKELYGPVDPTFLHRFYSLKAAVHGWKMVVCDKVRSLKLSD
NNCYLNAVIMTLDLLKDIKFVIPALQHAFMKHKGGDSTDFIALIMAYGNCTFGAPDDAS
RLLHTVLAKAELCCSARMVWREWCNVCGIKDVVLQGLKACCYVGVQTVEDLRARMT
YVCQCGGERHRQLVEHTTPWLLLSGTPNEKLVTTSTAPDFVAFNVFQGIETAVGHYVH
ARLKGGLILKFDSGTVSKTSDWKCKVTDVLFPGQKYSSDCNVVRYSLDGNFRTEVDPD
LSAFYVKDGKYFTSEPPVTYSPATILAGSVYTNSCLVSSDGQPGGDAISLSFNNLLGFDSS
KPVTKKYTYSFLPKEDGDVLLAEFDTYDPIYKNGAMYKGKPILWVNKASYDTNLNKFN
RASLRQIFDVAPIELENKFTPLSVESTPVEPPTVDVVALQQEMTIVKCKGLNKPFVKDNV
SFVADDSGTPVVEYLSKEDLHTLYVDPKYQVIVLKDNVLSSMLRLHTVESGDINVVAAS
GSLTRKVKLLFRASFYFKEFATRTFTATTAVGSCIKSVVRHLGVTKGILTGCFSFAKMLF
MLPLAYFSDSKLGTTEVKVSALKTAGVVTGNVVKQCCTAAVDLSMDKLRRVDWKSTL
RLLLMLCTTMVLLSSVYHLYVFNQVLSSDVMFEDAQGLKKFYKEVRAYLGISSACDGL
ASAYRANSFDVPTFCANRSAMCNWCLISQDSITHYPALKMVQTHLSHYVLNIDWLWFA
FETGLAYMLYTSAFNWLLLAGTLHYFFAQTSIFVDWRSYNYAVSSAFWLFTHIPMAGL
VRMYNLLACLWLLRKFYQHVINGCKDTACLLCYKRNRLTRVEASTVVCGGKRTFYITA
NGGISFCRRHNWNCVDCDTAGVGNTFICEEVANDLTTALRRPINATDRSHYYVDSVTV
KETVVQFNYRRDGQPFYERFPLCAFTNLDKLKFKEVCKTTTGIPEYNFIIYDSSDRGQES
LARSACVYYSQVLCKSILLVDSSLVTSVGDSSEIATKMFDSFVNSFVSLYNVTRDKLEKL
ISTARDGVRRGDNFHSVLTTFIDAARGPAGVESDVETNEIVDSVQYAHKHDIQITNESYN
NYVPSYVKPDSVSTSDLGSLIDCNAASVNQIVLRNSNGACIWNAAAYMKLSDALKRQIR
IACRKCNLAFRLTTSKLRANDNILSVRFTANKIVGGAPTWFNALRDFTLKGYVLATIIVF
LCAVLMYLCLPTFSMAPVEFYEDRILDFKVLDNGIIRDVNPDDKCFANKHRSFTQWYHE
HVGGVYDNSITCPLTVAVIAGVAGARIPDVPTTLAWVNNQIIFFVSRVFANTGSVCYTPI
DEIPYKSFSDSGCILPSECTMFRDAEGRMTPYCHDPTVLPGAFAYSQMRPHVRYDLYDG
NMFIKFPEVVFESTLRITRTLSTQYCRFGSCEYAQEGVCITTNGSWAIFNDHHLNRPGVY
CGSDFIDIVRRLAVSLFQPITYFQLTTSLVLGIGLCAFLTLLFYYINKVKRAFADYTQCAVI
AVVAAVLNSLCICFVTSIPLCIVPYTALYYYATFYFTNEPAFIMHVSWYIMFGPIVPIWMT
CVYTVAMCFRHFFWVLAYFSKKHVEVFTDGKLNCSFQDAASNIFVINKDTYAALRNSL
TNDAYSRFLGLFNKYKYFSGAMETAAYREAAACHLAKALQTYSETGSDLLYQPPNCSIT
SGVLQSGLVKMSHPSGDVEACMVQVTCGSMTLNGLWLDNTVWCPRHVMCPADQLSD
PNYDALLISMTNHSFSVQKHIGAPANLRVVGHAMQGTLLKLTVDVANPSTPAYTFTTV
KPGAAFSVLACYNGRPTGTFTVVMRPNYTIKGSFLCGSCGSVGYTKEGSVINFCYMHQ
MELANGTHTGSAFDGTMYGAFMDKQVHQVQLTDKYCSVNVVAWLYAAILNGCAWF
VKPNRTSVVSFNEWALANQFTEFVGTQSVDMLAVKTGVAIEQLLYAIQQLYTGFQGKQI
LGSTMLEDEFTPEDVNMQIMGVVMQSGVRKVTYGTAHWLFATLVSTYVIILQATKFTL
WNYLFETIPTQLFPLLFVTMAFVMLLVKHKHTFLTLFLLPVAICLTYANIVYEPTTPISSA
LIAVANWLAPTNAYMRTTHTDIGVYISMSLVLVIVVKRLYNPSLSNFALALCSGVMWL
YTYSIGEASSPIAYLVFVTTLTSDYTITVFVTVNLAKVCTYAIFAYSPQLTLVFPEVKMILL
LYTCLGFMCTCYFGVFSLLNLKLRAPMGVYDFKVSTQEFRFMTANNLTAPRNSWEAM
ALNFKLIGIGGTPCIKVAAMQSKLTDLKCTSVVLLSVLQQLHLEANSRAWAFCVKCHND
ILAATDPSEAFEKFVSLFATLMTFSGNVDLDALASDIFDTPSVLQATLSEFSHLATFAELE
AAQKAYQEAMDSGDTSPQVLKALQKAVNIAKNAYEKDKAVARKLERMADQAMTSM
YKQARAEDKKAKIVSAMQTMLFGMIKKLDNDVLNGIISNARNGCIPLSVIPLCASNKLR
VVIPDFTVWNQVVTYPSLNYAGALWDITVINNVDNEIVKSSDVVDSNENLTWPLVLECT
RASTSAVKLQNNEIKPSGLKTMVVSAGQEQTNCNTSSLAYYEPVQGRKMLMALLSDNA
YLKWARVEGKDGFVSVELQPPCKFLIAGPKGPEIRYLYFVKNLNNLHRGQVLGHIAATV
RLQAGSNTEFASNSSVLSLVNFTVDPQKAYLDFVNAGGAPLTNCVKMLTPKTGTGIAIS
VKPESTADQETYGGASVCLYCRAHIEHPDVSGVCKYKGKFVQIPAQCVRDPVGFCLSNT
PCNVCQYWIGYGCNCDSLRQAALPQSKDSNFLNRVRGSIVNARIEPCSSGLSTDVVFRA
FDICNYKAKVAGIGKYYKTNTCRFVELDDQGHHLDSYFVVKRHTMENYELEKHCYDLL
RDCDAVAPHDFFIFDVDKVKTPHIVRQRLTEYTMMDLVYALRHFDQNSEVLKAILVKY
GCCDVTYFENKLWFDFVENPSVIGVYHKLGERVRQAILNTVKFCDHMVKAGLVGVLTL
DNQDLNGKWYDFGDFVITQPGSGVAIVDSYYSYLMPVLSMTDCLAAETHRDCDFNKPL
IEWPLTEYDFTDYKVQLFEKYFKYWDQTYHANCVNCTDDRCVLHCANFNVLFAMTMP
KTCFGPIVRKIFVDGVPFVVSCGYHYKELGLVMNMDVSLHRHRLSLKELMMYAADPA
MHIASSNAFLDLRTSCFSVAALTTGLTFQTVRPGNFNQDFYDFVVSKGFFKEGSSVTLKH
FFFAQDGNAAITDYNYYSYNLPTMCDIKQMLFCMEVVNKYFEIYDGGCLNASEVVVNN
LDKSAGHPFNKFGKARVYYESMSYQEQDELFAMTKRNVIPTMTQMNLKYAISAKNRA
RTVAGVSILSTMTNRQYHQKMLKSMAATRGATCVIGTTKFYGGWDFMLKTLYKDVDN
PHLMGWDYPKCDRAMPNMCRIFASLILARKHGTCCTTRDRFYRLANECAQVLSEYVLC
GGGYYVKPGGTSSGDATTAYANSVFNILQATTANVSALMGANGNKIVDKEVKDMQFD
LYVNVYRSTSPDPKFVDKYYAFLNKHFSMMILSDDGVVCYNSDYAAKGYIAGIQNFKE
TLYYQNNVFMSEAKCWVETDLKKGPHEFCSQHTLYIKDGDDGYFLPYPDPSRILSAGCF
VDDIVKTDGTLMVERFVSLAIDAYPLTKHEDIEYQNVFWVYLQYIEKLYKDLTGHMLD
SYSVMLCGDNSAKFWEEAFYRDLYSSPTTLQAVGSCVVCHSQTSLRCGTCIRRPFLCCK
CCYDHVIATPHKMVLSVSPYVCNAPGCGVSDVTKLYLGGMSYFCVDHRPVCSFPLCAN
GLVFGLYKNMCTGSPSIVEFNRLATCDWTESGDYTLANTTTEPLKLFAAETLRATEEAS
KQSYAIATIKEIVGERQLLLVWEAGKSKPPLNRNYVFTGYHITKNSKVQLGEYIFERIDYS
DAVSYKSSTTYKLTVGDIFVLTSHSVATLTAPTIVNQERYVKITGLYPTITVPEEFASHVA
NFQKSGYSKYVTVQGPPGTGKSHFAIGLAIYYPTARVVYTACSHAAVDALCEKAFKYL
NIAKCSRIIPAKARVECYDRFKVNETNSQYLFSTINALPETSADILVVDEVSMCTNYDLSII
NARIKAKHIVYVGDPAQLPAPRTLLTRGTLEPENFNSVTRLMCNLGPDIFLSMCYRCPKE
IVSTVSALVYNNKLLAKKELSGQCFKILYKGNVTHDASSAINRPQLTFVKNFITANPAWS
KAVFISPYNSQNAVSRSMLGLTTQTVDSSQGSEYQYVIFCQTADTAHANNINRFNVAITR
AQKGILCVMTSQALFESLEFTELSFTNYKLQSQIVTGLFKDCSRETSGLSPAYAPTYVSV
DDKYKTSDELCVNLNLPANVPYSRVISRMGFKLDATVPGYPKLFITREEAVRQVRSWIG
FDVEGAHASRNACGTNVPLQLGFSTGVNFVVQPVGVVDTEWGNMLTGIAARPPPGEQF
KHLVPLMHKGAAWPIVRRRIVQMLSDTLDKLSDYCTFVCWAHGFELTSASYFCKIGKE
QKCCMCNRRAAAYSSPLQSYACWTHSCGYDYVYNPFFVDVQQWGYVGNLATNHDRY
CSVHQGAHVASNDAIMTRCLAIHSCFIERVDWDIEYPYISHEKKLNSCCRIVERNVVRAA
LLAGSFDKVYDIGNPKGIPIVDDPVVDWHYFDAQPLTRKVQQLFYTEDMASRFADGLC
LFWNCNVPKYPNNAIVCRFDTRVHSEFNLPGCDGGSLYVNKHAFHTPAYDVSAFRDLK
PLPFFYYSTTPCEVHGNGSMIEDIDYVPLKSAVCITACNLGGAVCRKHATEYREYMEAY
NLVSASGFRLWCYKTFDIYNLWSTFTKVQGLENIAFNVVKQGHFIGVEGELPVAVVND
KIFTKSGVNDICMFENKTTLPTNIAFELYAKRAVRSHPDFKLLHNLQADICYKFVLWDYE
RSNIYGTATIGVCKYTDIDVNSALNICFDIRDNCSLEKFMSTPNAIFISDRKIKKYPCMVG
PDYAYFNGAIIRDSDVVKQPVKFYLYKKVNNEFIDPTECIYTQSRSCSDFLPLSDMEKDF
LSFDSDVFIKKYGLENYAFEHVVYGDFSHTTLGGLHLLIGLYKKQQEGHIIMEEMLKGSS
TIHNYFITETNTAAFKAVCSVIDLKLDDFVMILKSQDLGVVSKVVKVPIDLTMIEFMLWC
KDGQVQTFYPRLQASADWKPGHAMPSLFKVQNVNLERCELANYKQSIPMPRGVHMNI
AKYMQLCQYLNTCTLAVPANMRVIHFGAGSDKGIAPGTSVLRQWLPTDAIIIDNDLNEF
VSDADITLFGDCVTVRVGQQVDLVISDMYDPTTKNVTGSNESKALFFTYLCNLINNNLA
LGGSVAIKITEHSWSVELYELMGKFAWWTVFCTNANASSSEGFLLGINYLGTIKENIDGG
AMHANYIFWRNSTPMNLSTYSLFDLSKFQLKLKGTPVLQLKESQINELVISLLSQGKLLI
RDNDTLSVSTDVLVNTYRKLR (SEQ ID NO: 3)
HKU1 (beta coronavirus); NCBI GenBank accession code KF686346:
MIKTSKYGLGFKWAPEFRWLLPDAAEELASPMKSDEGGLCPSTGQAMESVGFVYDNH
VKIDCRCILGQEWHVQSNLIRDIFVHEDLHVVEVLTKTAVKSGTAILIKSPLHSLGGFPKG
YVMGLFRSYKTKRYVVHHLSMTTSTTNFGEDFLGWIVPFGFMPSYVHKWFQFCRLYIE
ESDLIISNFKFDDYDFSVEDVYAEVHAEPKGKYSQKAYALLRQYRGIKPVLFVDQYGCD
YSGKLADCLQAYGHYSLQDMRQKQSVWLANCDFDIVVAWHVVRDSRFVMRLQTIATI
CGIKYVAQPTEDVVDGDVVIREPVHLLSADAIVLKLPSLMKVMTHMDDFSIKSIYNVDL
CDCGFVMQYGYVDCFNDNCDFYGWVSGNMMDGFSCPLCCTVYDSSEVKAQSSGVIPE
NPVLFTNSTDTVNHDSFNLYGYSVTPFGSCIYWSPRPGLWIPIIKSSVKSYDDLVYSGVV
GCKSIVKETALITHALYLDYVQCKCGNLEQNHILGVNNSWCRQLLLNRGDYNMLLKNI
DLFVKRRADFACKFAVCGDGFVPFLLDGLIPRSYYLIQSGIFFTSLMSQFSQEVSDMCLK
MCILFMDRVSVATFYIEHYVNRLVTQFKLLGTTLVNKMVNWFNTMLDASAPATGWLL
YQLLNGLFVVSQANFNFVALIPDYAKILVNKFYTFFKLLLECVTVDVLKDMPVLKTING
LVCIVGNKFYNVSTGLIPGFVLPCNAQEQQIYFFEGVAESVIVEDDVIENVKSSLSSYEYC
QPPKSVEKICIIDNMYMGKCGDKFFPIVMNDKNICLLDQAWRFPCAGRKVNFNEKPVV
MEIPSLMTVKVMFDLDSTFDDILGKVCSEFEVEKGVTVDDFVAVVCDAIENALNSCKEH
PVVGYQVRAFLNKLNDNVVYLFDEAGDEAMASRMYCTFAIEDVEDVISSEAVEDTIDGI
VEDTINDDEDVVTGDNDDEDVVTGDNDDEDVVTGDNDDEDVVTGDNDDEDVVTGDN
DDEDVVTGDNDDEDVVTGDNDDEDVVTGDNDDEDVVTGDNDDEDVVTGDNDDEDV
VTGDNDDEDVVTGDNDDEDVVTGDNDDEDVVTGDNDDEDVVTGDNDDEDVVTGDN
DDEDVVTGDNDDEDNNDEEIVTGDNDDQIVVTGDDVDDIESIYDFDTYKALLVFNDVY
NDALFVSYGSSVETETYFKVNGLWSPTITHTNCWLRSVLLVMQKLPFKFKDLAIENMW
LSYKVGYNQSFVDYLLTTIPKAIVLPQGGFVADFAYWFLNQFDINAYANWCCLKCGFSF
DLNGLDALFFYGDIVSHVCKCGHNMTLIAADLPCTLHFSLFDDNFCAFCTPKKIFIAACA
VDVNVCHSVAVIGDEQIDGKFVTKFSGDKFDFIVGYGMSFSMSSFELAQLYGLCITPNV
CFVKGDIINVARLVKADVIVNPANGHMLHGGGVAKAIAVAAGKKFSKETAAMVKSKG
VCQVGDCYVSTGGKLCKTILNIVGPDARQDGRQSYVLLARAYKHLNNYDCCLSTLISA
GIFSVPADVSLTYLLGVVDKQVILVSNNKEDFDIIQKCQITSVVGTKALAVRLTANVGRV
IKFETDAYKLFLSGDDCFVSNSSVIQEVLLLRHDIQLNNDVRDYLLSKMTSLPKDWRLIN
KFDVINGVKTVKYFECPNSIYICSQGKDFGYVCDGSFYKATVNQVCVLLAKKIDVLLTV
DGVNFKSISLTVGEVFGKILGNVFCDGIDVTKLKCSDFYADKILYQYENLSLADISAVQS
SFGFDQQQLLAYYNFLTVCKWSVVVNGPFFSFEQSHNNCYVNVACLMLQHINLKFNK
WQWQEAWYEFRAGRPHRLVALVLAKGHFKFDEPSDATDFIRVVLKQADLSGAICELEL
ICDCGIKQESRVGVDAVMHFGTLAKTDLFNGYKIGCNCAGRIVHCTKLNVPFLICSNTPL
SKDLPDDVVAANMFMGVGVGHYTHLKCGSPYQHYDACSVKKYTGVSGCLTDCLYLK
NLTQTFTSMLTNYFLDDVEMVAYNPDLSQYYCDNGKYYTKPIIKAQFKPFAKVDGVYT
NFKLVGHDICAQLNDKLGFNVDLPFVEYKVTVWPVATGDVVLASDDLYVKRYFKGCE
TFGKPVIWFCHDEASLNSLTYFNKPSFKSENRYSVLSVDSVSEESQGNVVTPVMESQIST
KEVKLKGVRKTVKIEDAIIVNDENSSIKVVKSLSLVDVWDMYLTGCDYVVWVANELSR
LVKSPTVREYIRYGIKPITIPIDLLCLRDDNQTLLVPKIFKARAIEFYGFLKWLFIYVFSLLH
FTNDKTIFYTTEIASKFTFNLFCLALKNAFQTFRWSIFIKGFLVVATVFLFWFNFLYINVIF
SDFYLPNISVFPIFVGRIVMWIKATFGLVTICDFYSKLGVGFTSHFCNGSFICELCHSGFD
MLDTYAAIDFVQYEVDRRVLFDYVSLVKLIVELVIGYSLYTVWFYPLFCLIGLQLFTTWL
PDLFMLETMHWLIRFIVFVANMLPAFVLLRFYIVVTAMYKVVGFIRHIVYGCNKAGCLF
CYKRNCSVRVKCSTIVGGVIRYYDITANGGTGFCVKHQWNCFNCHSFKPGNTFITVEAA
IELSKELKRPVNPTDASHYVVTDIKQVGCMMRLFYDRDGQRVYDDVDASLFVDINNLL
HSKVKVVPNLYVVVVESDADRANFLNAVVFYAQSLYRPILLVDKKLITTACNGISVTQT
MFDVYVDTFMSHFDVDRKSFNNFVNIAHASLREGVQLEKVLDTFVGCVRKCCSIDSDV
ETRFITKSMISAVAAGLEFTDENYNNLVPTYLKSDNIVAADLGVLIQNGAKHVQGNVAK
AANISCIWFIDAFNQLTADLQHKLKKACVKTGLKLKLTFNKQEASVPILTTPFSLKGGVV
LSNLLYILFFVSLICFILLWALLPTYSVYKSDIHLPAYASFKVIDNGVVRDISVNDLCFAN
KFFQFDQWYESTFGSVYYHNSMDCPIVVAVMDEDIGSTMFNVPTKVLRHGFHVLHFLT
YAFASDSVQCYTPHIQISYNDFYASGCVLSSLCTMFKRGDGTPHPYCYSDGVMKNASLY
TSLVPHTRYSLANSNGFIRFPDVISEGIVRIVRTRSMTYCRVGACEYAEEGICFNFNSSWV
LNNDYYRSMPGTFCGRDLFDLFYQFFSSLIRPIDFFSLTASSIFGAILAIVVVLVFYYLIKL
KRAFGDYTSVVVINVVVWCINFLMLFVFQVYPICACVYACFYFYVTLYFPSEISVIMHLQ
WIVMYGAIMPFWFCVTYVAMVIANHVLWLFSYCRKIGVNVCSDSTFEETSLTTFMITKD
SYCRLKNSVSDVAYNRYLSLYNKYRYYSGKMDTAAYREAACSQLAKAMETFNHNNG
NDVLYQPPTASVSTSFLQSGIVKMVSPTSKIEPCIVSVTYGSMTLNGLWLDDKVYCPRH
VICSSSNMNEPDYSALLCRVTLGDFTIMSGRMSLTVVSYQMQGCQLVLTVSLQNPYTPK
YTFGNVKPGETFTVLAAYNGRPQGAFHVTMRSSYTIKGSFLCGSCGSVGYVLTGDSVKF
VYMHQLELSTGCHTGTDFTGNFYGPYRDAQVVQLPVKDYVQTVNVIAWLYAAILNNC
AWFVQNDVCSTEDFNVWAMANGFSQVKADLVLDALASMTGVSIETLLAAIKRLYMGF
QGRQILGSCTFEDELAPSDVYQQLAGVKLQSKTKRFIKETIYWILISTFLFSCIISAFVKWT
IFMYINTHMIGVTLCVLCFVSFMMLLVKHKHFYLTMYIIPVLCTLFYVNYLVVYKEGFR
GFTYVWLSYFVPAVNFTYVYEVFYGCILCVFAIFITMHSINHDIFSLMFLVGRIVTLISMW
YFGSNLEEDVLLFITAFLGTYTWTTILSLAIAKIVANWLSVNIFYFTDVPYIKLILLSYLFIG
YILSCYWGFFSLLNSVFRMPMGVYNYKISVQELRYMNANGLRPPRNSFEAILLNLKLLGI
GGVPVIEVSQIQSKLTDVKCANVVLLNCLQHLHVASNSKLWQYCSVLHNEILSTSDLSV
AFDKLAQLLIVLFANPAAVDTKCLASIDEVSDDYVQDSTVLQALQSEFVNMASFVEYEV
AKKNLADAKNSGSVNQQQIKQLEKACNIAKSVYERDKAVARKLERMADLALTNMYKE
ARINDKKSKVVSALQTMLFSMVRKLDNQALNSILDNAVKGCVPLSAIPALAANTLTIIIP
DKQVFDKVVDNVYVTYAGSVWHIQTVQDADGINKQLTDISVDSNWPLVIIANRYNEVA
NAVMQNNELMPHKLKIQVVNSGSDMNCNIPTQCYYNNGSSGRIVYAVLSDVDGLKYT
KIMKDDGNCVVLELDPPCKFSIQDVKGLKIKYLYFIKGCNTLARGWVVGTLSSTIRLQA
GVATEYAANSSILSLCAFSVDPKKTYLDYIQQGGVPIINCVKMLCDHAGTGMAITIKPEA
TINQDSYGGASVCIYCRARVEHPDVDGICKLRGKFVQVPLGIKDPILYVLTHDVCQVCG
FWRDGSCSCVGSSVAVQSKDLNFLNRVRGTSVNARLVPCASGLSTDVQLRAFDICNTN
RAGIGLYYKVNCCRFQRIDDDGNKLDKFFVVKRTNLEVYNKEKTYYELTKSCGVVAEH
DFFTFDIDGSRVPHIVRRNLSKYTMLDLCYALRHFDRNDCSILCEILCEYADCKESYFSK
KDWYDFVENPDIINIYKKLGPIFNRALLNTVIFADTLVEVGLVGVLTLDNQDLYGQWYD
FGDFIQTAPGFGVAVADSYYSYMMPMLTMCHVLDCELFVNDSYRQFDLVQYDFTDYK
LELFNKYFKYWGMKYHPNTVDCDNDRCIIHCANFNILFSMVLPNTCFGPLVRQIFVDGV
PFVVSIGYHYKELGVVMNLDVDTHRYRLSLKDLLLYAADPAMHVASASALLDLRTCCF
SVAAITSGIKFQTVKPGNFNQDFYEFVKSKGLFKEGSTVDLKHFFFTQDGNAAITDYNY
YKYNLPTMVDIKQLLFVLEVVYKYFEIYDGGCIPASQVIVNNYDKSAGYPFNKFGKARL
YYEALSFEEQNEIYAYTKRNVLPTLTQMNLKYAISAKNRARTVAGVSILSTMTGRMFHQ
KCLKSIAATRGVPVVIGTTKFYGGWDDMLRHLIKDVDNPVLMGWDYPKCDRAMPNIL
RIVSSLVLARKHEFCCSHGDRFYRLANECAQVLSEIVMCGGCYYVKPGGTSSGDATTAF
ANSVFNICQAVTANVCSLMACNGHKIEDLSIRNLQKRLYSNVYRTDYVDYTFVNEYYE
FLCKHFSMMILSDDGVVCYNSDYASKGYIANISVFQQVLYYQNNVFMSESKCWVENDI
TNGPHEFCSQHTMLVKIDGDYVYLPYPDPSRILGAGCFVDDLLKTDSVLLIERFVSLAID
AYPLVYHENEEYQKVFRVYLEYIKKLYNDLGTQILDSYSVILSTCDGLKFTEESFYKNM
YLKSAVMQSVGACVVCSSQTSLRCGSCIRKPLLCCKCCYDHVMATNHKYVLSVSPYVC
NAPNCDVSDVTKLYLGGMSYYCENHKPHYSFKLVMNGMVFGLYKQSCTGSPYIDDFN
KIASCKWTEVDDYVLANECIERLKLFAAETQKATEEAFKQSYASATIQEIVSDREVILCW
ETGKVKPPLNKNYVFTGYHFTSTGKTVLGEYVFDKSELTNGVYYRATTTYKLSIGDVFV
LTSHSVASLSAPTLVPQENYASIRFSSVYSVPLVFQNNVANYQHIGMKRYCTVQGPPGT
GKSHLAIGLAVYYYTARVVYTAASHAAVDALCEKAYKFLNINDCTRIIPAKVRVDCYD
KFKINDTTCKYVFTTINALPELVTDIVVVDEVSMLTNYELSVINARIKAKHYVYIGDPAQ
LPAPRVLLSKGSLEPRHFNSITKIMCCLGPDIFLGNCYRCPKEIVETVSALVYDNKLKAK
NDNSSLCFKVYFKGQTTHESSSAVNIQQIYLISKFLKANPVWNSAVFISPYNSQNYVAKR
VLGVQTQTVDSAQGSEYDYVIYSQTAETAHSVNVNRFNVAITRAKKGIFCVMSNMQLF
ESLNFITLPLDKIQNQTLPRLHCTTNLFKDCSKSCLGYHPAHAPSFLAVDDKYKVNENLA
VNLNICEPVLTYSRLISLMGFKLDLTLDGYSKLFITKDEAIKRVRGWVGFDVEGAHATRE
NIGTNFPLQIGFSTGVDFVVEATGLFAERDCYTFKKTVAKAPPGEKFKHLIPLMSKGQK
WDIVRIRIVQMLSDYLLDLSDSVVFITWSASFELTCLRYFAKLGRELNCNVCSNRATCYN
SRTGYYGCWRHSYTCDYVYNPLIVDIQQWGYTGSLTSNHDIICNVHKGAHVASADAIM
TRCLAIYDCFCKSVNWNLEYPIISNEVSINTSCRLLQRVMLKAAMLCNRYNLCYDIGNP
KGLACVKDYEFKFYDAFPVAKFVKQLFYVYDVHKDNFKDGLCMFWNCNVDKYPSNSI
VCRFDTRVLNKLNLPGCNGGSLYVNKHAFHTNPFTRTVFENLKPMPFFYYSDTPCVYV
DGLESKQVDYVPLRSATCITRCNLGGAVCSKHAEEYCNYLESYNIVTTAGFTFWVYKNF
DFYNLWNTFTTLQSLENVIYNLVNVGHYDGRTGELPCAIINDKVVVKINNVDTVIFKNN
TSFPTNIAVELFTKRSIRHHPELKILRNLNIDICWKHVLWDYVKDSLFCSSTYGVCKYTDL
KFIENLNILFDGRDTGALEAFRKARNGVFISTEKLSRLSMIKGPQRADLNGVIVDKVGEL
KVEFWFAMRKDGDDVIFSRTDSLCSSHYWSPQGNLGGNCAGNVIGNDALTRFTIFTQSR
VLSSFEPRSDLERDFIDMDDNLFIAKYGLEDYAFDHIVYGSFNHKVIGGLHLLIGLFRRLK
KSNLLIQEFLQYDSSIHSYFITDQECGSSKSVCTVIDLLLDDFVSIVKSLNLSCVSKVVNIN
VDFKDFQFMLWCNDNKIMTFYPKMQATNDWKPGYSMPVLYKYLNVPLERVSLWNYG
KPINLPTGCMMNVAKYTQLCQYLNTTTLAVPVNMRVLHLGAGSDKEVAPGSAVLRQW
LPSGSILVDNDLNPFVSDSLVTYFGDCMTLPFDCHWDLIISDMYDPLTKNIGDYNVSKDG
FFTYICHLIRDKLSLGGSVAIKITEFSWNADLYKLMSCFAFWTVFCTNVNASSSEGFLIGI
NYLGKSSFEIDGNVMHANYLFWRNSTTWNGGAYSLFDMTKFSLKLAGTAVVNLRPDQ
LNDLVYSLIERGKLLVRDTRKEIFVGDSLVNTC (SEQ ID NO: 4)
OC43 (beta coronavirus); NCBI GenBank accession code NC_006213:
MSKINKYGLELHWAPEFPWMFEDAEEKLDNPSSSEVDMICSTTAQKLETDGICPENHVM
VDCRRLLKQECCVQSSLIREIVMNASPYDLEVLLQDALQSREAVLVTTPLGMSLEACYV
RGCNPKGWTMGLFRRRSVCNTGRCTVNKHVAYQLYMIDPAGVCLGAGQFVGWVIPLA
FMPVQSRKFIVPWVMYLRKRGEKGAYNKDHGRGGFGHVYDFKVEDAYDQVHDEPKG
KFSKKAYALIRGYRGVKPLLYVDQYGCDYTGSLADGLEAYADKTLQEMKALFPTWSQ
ELLFDVIVAWHVVRDPRYVMRLQSAATIRSVAYVANPTEDLCDGSVVIKEPVHVYADD
SIILRQYNLVDIMSHFYMEADTVVNAFYGVALKDCGFVMQFGYIDCEQDSCDFKGWIP
GNMIDGFACTTCGHVYEVGDLMAQSSGVLPVNPVLHTKSAAGYGGFGCKDSFTLYGQ
TVVYFGGCVYWSPARNIWIPILKSSVKSYDSLVYTGVLGCKAIVKETNLICKALYLDYV
QHKCGNLHQRELLGVSDVWHKQLLLNRGVYKPLLENIDYFNMRRAKFSLETFTVCADG
FMPFLLDDLVPRAYYLAVSGQAFCDYADKLCHAVVSKSKELLDVSLDSLGAAIHYLNS
KIVDLAQHFSDFGTSFVSKIVHFFKTFTTSTALAFAWVLFHVLHGAYIVVESDIYFVKNIP
RYASAVAQAFQSVAKVVLDSLRVTFIDGLSCFKIGRRRICLSGRKIYEVERGLLHSSQLPL
DVYDLTMPSQVQKAKQKPIYLKGSGSDFSLADSVVEVVTTSLTPCGYSEPPKVAAKICIV
DNVYMAKAGDKYYPVVVDDHVGLLDQAWRVPCAGRRVTFKEQPTVKEIISMPKIIKVF
YELDNDFNTILNTACGVFEVDDTVDMEEFYAVVIDAIEEKLSPCKELEGVGAKVSAFLQ
KLEDNPLFLFDEAGEEVLAPKLYCAFTAPEDDDFLEESDVEEDDVEGEETDLTVTSAGQ
PCVASEQEESSEVLEDTLDDGPSVETSDSQVEEDVEMSDFVDLESVIQDYENVCFEFYTT
EPEFVKVLGLYVPKATRNNCWLRSVLAVMQKLPCQFKDKNLQDLWVLYKQQYSQLFV
DTLVNKIPANIVLPQGGYVADFAYWFLTLCDWQCVAYWKCIKCDLALKLKGLDAMFF
YGDVVSHICKCGESMVLIDVDVPFTAHFALKDKLFCAFITKRIVYKAACVVDVNDSHSM
AVVDGKQIDDHRITSITSDKFDFIIGHGMSFSMTTFEIAQLYGSCITPNVCFVKGDIIKVSK
LVKAEVVVNPANGHMAHGGGVAKAIAVAAGQQFVKETTDMVKSKGVCATGDCYVST
GGKLCKTVLNVVGPDARTQGKQSYVLLERVYKHLNNYDCVVTTLISAGIFSVPSDVSLT
YLLGTAKKQVVLVSNNQEDFDLISKCQITAVEGTKKLAARLSFNVGRSIVYETDANKLIL
INDVAFVSTFNVLQDVLSLRHDIALDDDARTFVQSNVDVVPEGWRVVNKFYQINGVRT
VKYFECTGGIDICSQDKVFGYVQQGIFNKATVAQIKALFLDKVDILLTVDGVNFTNRFVP
VGESFGKSLGNVFCDGVNVTKHKCDINYKGKVFFQFDNLSSEDLKAVRSSFNFDQKELL
AYYNMLVNCFKWQVVVNGKYFTFKQANNNCFVNVSCLMLQSLHLTFKIVQWQEAWL
EFRSGRPARFVALVLAKGGFKFGDPADSRDFLRVVFSQVDLTGAICDFEIACKCGVKQE
QRTGLDAVMHFGTLSREDLEIGYTVDCSCGKKLIHCVRFDVPFLICSNTPASVKLPKGVG
SANIFIGDKVGHYVHVKCEQSYQLYDASNVKKVTDVTGKLSDCLYLKNLKQTFKSVLT
TYYLDDVKKIEYKPDLSQYYCDGGKYYTQRIIKAQFKTFEKVDGVYTNFKLIGHTVCDS
LNAKLGFDSSKEFVEYKITEWPTATGDVVLATDDLYVKRYERGCITFGKPVIWLSHEKA
SLNSLTYFNRPSLVDDNKFDVLKVDDVDDGGDSSESGAKETKEINIIKLSGVKKPFKVED
SVIVNDDTSETKYVKSLSIVDVYDMWLTGCKYVVRTANALSRAVNVPTIRKFIKFGMTL
VSIPIDLLNLREIKPAVNVVKAVRNKISVCFNFIKWLFVLLFGWIKISADNKVIYTTETASK
LTCKLVALAFKNAFLTFKWSMVARGACIIATIFLLWFNFIYANVIFSDFYLPKIGFLPTFV
GKIAQWIKNTFSLVTICDLYSIQDVGFKNQYCNGSIACQFCLAGFDMLDNYKAIDVVQY
EADRRAFVDYTGVLKIVIELIVSYALYTAWFYPLFALISIQILTTWLPELFMLSTLHWSFR
LLVALANMLPAHVFMRFYIIIASFIKLFSLFRHVAYGCSKSGCLFCYKRNRSLRVKCSTIV
GGMIRYYDVMANGGTGFCSKHQWNCIDCDSYKPGNTFITVEAALDLSKELKRPIQPTD
VAYHTVTDVKQVGCSMRLFYDRDGQRTYDDVNASLFVDYSNLLHSKVKSVPNMHVV
VVENDADKANFLNAAVFYAQSLFRPILMVDKNLITTANTGTSVTETMFDVYVDTFLSM
FDVDKKSLNALIATAHSSIKQGTQIYKVLDTFLSCARKSCSIDSDVDTKCLADSVMSAVS
AGLELTDESCNNLVPTYLKSDNIVAADLGVLIQNSAKHVQGNVAKIAGVSCIWSVDAFN
QFSSDFQHKLKKACCKTGLKLKLTYNKQMANVSVLTTPFSLKGGAVFSYFVYVCFVLS
LVCFIGLWCLMPTYTVHKSDFQLPVYASYKVLDNGVIRDVSVEDVCFANKFEQFDQWY
ESTFGLSYYSNSMACPIVVAVIDQDFGSTVFNVPTKVLRYGYHVLHFITHALSADGVQC
YTPHSQISYSNFYASGCVLSSACTMFTMADGSPQPYCYTEGLMQNASLYSSLVPHVRYN
LANAKGFIRFPEVLREGLVRIVRTRSMSYCRVGLCEEADEGICFNFNGSWVLNNDYYRS
LPGTFCGRDVFDLIYQLFKGLAQPVDFLALTASSIAGAILAVIVVLVFYYLIKLKRAFGDY
TSVVFVNVIVWCVNFMMLFVFQVYPILSCVYAICYFYATLYFPSEISVIMHLQWLVMYG
TIMPLWFCLLYIAVVVSNHAFWVFSYCRKLGTSVRSDGTFEEMALTTFMITKDSYCKLK
NSLSDVAFNRYLSLYNKYRYYSGKMDTAAYREAACSQLAKAMDTFTNNNGSDVLYQP
PTASVSTSFLQSGIVKMVNPTSKVEPCVVSVTYGNMTLNGLWLDDKVYCPRHVICSASD
MTNPDYTNLLCRVTSSDFTVLFDRLSLTVMSYQMRGCMLVLTVTLQNSRTPKYTFGVV
KPGETFTVLAAYNGKPQGAFHVTMRSSYTIKGSFLCGSCGSVGYVIMGDCVKFVYMHQ
LELSTGCHTGTDFNGDFYGPYKDAQVVQLLIQDYIQSVNFVAWLYAAILNNCNWFVQS
DKCSVEDFNVWALSNGFSQVKSDLVIDALASMTGVSLETLLAAIKRLKNGFQGRQIMGS
CSFEDELTPSDVYQQLAGIKLQSKRTRLFKGTVCWIMASTFLFSCIITAFVKWTMFMYVT
TNMFSITFCALCVISLAMLLVKHKHLYLTMYITPVLFTLLYNNYLVVYKHTFRGYVYA
WLSYYVPSVEYTYTDEVIYGMLLLVGMVFVTLRSINHDLFSFIMFVGRLISVFSLWYKG
SNLEEEILLMLASLFGTYTWTTVLSMAVAKVIAKWVAVNVLYFTDIPQIKIVLLCYLFIG
YIISCYWGLFSLMNSLFRMPLGVYNYKISVQELRYMNANGLRPPKNSFEALMLNFKLLG
IGGVPIIEVSQFQSKLTDVKCANVVLLNCLQHLHVASNSKLWHYCSTLHNEILATSDLSV
AFEKLAQLLIVLFANPAAVDSKCLTSIEEVCDDYAKDNTVLQALQSEFVNMASFVEYEV
AKKNLDEARFSGSANQQQLKQLEKACNIAKSAYERDRAVAKKLERMADLALTNMYKE
ARINDKKSKVVSALQTMLFSMVRKLDNQALNSILDNAVKGCVPLNAIPSLAANTLNIIVP
DKSVYDQVVDNVYVTYAGNVWQIQTIQDSDGTNKQLNEISDDCNWPLVIIANRYNEVS
ATVLQNNELMPAKLKIQVVNSGPDQTCNTPTQCYYNNSNNGKIVYAILSDVDGLKYTKI
LKDDGNFVVLELDPPCKFTVQDAKGLKIKYLYFVKGCNTLARGWVVGTISSTVRLQAG
TATEYASNSSILSLCAFSVDPKKTYLDFIQQGGTPIANCVKMLCDHAGTGMAITVKPDAT
TSQDSYGGASVCIYCRARVEHPDVDGLCKLRGKFVQVPVGIKDPVSYVLTHDVCRVCG
FWRDGSCSCVSTDTTVQSKDTNFLNRVRGTSVDARLVPCASGLSTDVQLRAFDIYNASV
AGIGLHLKVNCCRFQRVDENGDKLDQFFVVKRTDLTIYNREMKCYERVKDCKFVAEH
DFFTFDVEGSRVPHIVRKDLTKYTMLDLCYALRHFDRNDCMLLCDILSIYAGCEQSYFT
KKDWYDFVENPDIINVYKKLGPIFNRALVSATEFADKLVEVGLVGVLTLDNQDLNGKW
YDFGDYVIAAPGCGVAIADSYYSYIMPMLTMCHALDCELYVNNAYRLFDLVQYDFTDY
KLELFNKYFKHWSMPYHPNTVDCQDDRCIIHCANFNILFSMVLPNTCFGPLVRQIFVDG
VPFVVSIGYHYKELGIVMNMDVDTHRYRLSLKDLLLYAADPALHVASASALYDLRTCC
FSVAAITSGVKFQTVKPGNFNQDFYDFVLSKGLLKEGSSVDLKHFFFTQDGNAAITDYN
YYKYNLPTMVDIKQLLFVLEVVYKYFEIYDGGCIPASQVIVNNYDKSAGYPFNKFGKAR
LYYEALSFEEQDEIYAYTKRNVLPTLTQMNLKYAISAKNRARTVAGVSILSTMTGRMFH
QKCLKSIAATRGVPVVIGTTKFYGGWDDMLRRLIKDVDNPVLMGWDYPKCDRAMPNL
LRIVSSLVLARKHETCCSQSDRFYRLANECAQVLSEIVMCGGCYYVKPGGTSSGDATTA
FANSVFNICQAVSANVCALMSCNGNKIEDLSIRALQKRLYSHVYRSDKVDSTFVTEYYE
FLNKHFSMMILSDDGVVCYNSDYASKGYIANISAFQQVLYYQNNVFMSESKCWVEHDI
NNGPHEFCSQHTMLVKMDGDDVYLPYPNPSRILGAGCFVDDLLKTDSVLLIERFVSLAI
DAYPLVYHENEEYQKVFRVYLAYIKKLYNDLGNQILDSYSVILSTCDGQKFTDESFYKN
MYLRSAVMQSVGACVVCSSQTSLRCGSCIRKPLLCCKCCYDHVMATDHKYVLSVSPYV
CNAPGCDVNDVTKLYLGGMSYYCEDHKPQYSFKLVMNGLVFGLYKQSCTGSPYIDDF
NRIASCKWTDVDDYILANECTERLKLFAAETQKATEEAFKQSYASATIQEIVSERELILS
WEIGKVKPPLNKNYVFTGYHFTKNGKTVLGEYVFDKSELTNGVYYRATTTYKLSVGDV
FVLTSHSVANLSAPTLVPQENYSSIRFASVYSVLETFQNNVVNYQHIGMKRYCTVQGPP
GTGKSHLAIGLAVFYCTARVVYTAASHAAVDALCEKAYKFLNINDCTRIVPAKVRVEC
YDKFKINDTTRKYVFTTINALPEMVTDIVVVDEVSMLTNYELSVINARIRAKHYVYIGDP
AQLPAPRVLLSKGTLEPKYFNTVTKLMCCLGPDIFLGTCYRCPKEIVDTVSALVYENKL
KAKNESSSLCFKVYYKGVTTHESSSAVNMQQIYLINKFLKANPLWHKAVFISPYNSQNF
AAKRVLGLQTQTVDSAQGSEYDYVIYSQTAETAHSVNVNRFNVAITRAKKGILCVMSN
MQLFEALQFTTLTLDKVPQAVETKVQCSTNLFKDCSKSYSGYHPAHAPSFLAVDDKYK
ATGDLAVCLGIGDSAVTYSRLISLMGFKLDVTLDGYCKLFITKEEAVKRVRAWVGFDAE
GAHATRDSIGTNFPLQLGFSTGIDFVVEATGLFADRDGYSFKKAVAKAPPGEQFKHLIPL
MTRGHRWDVVRPRIVQMFADHLIDLSDCVVLVTWAANFELTCLRYFAKVGREISCNVC
TKRATVYNSRTGYYGCWRHSVTCDYLYNPLIVDIQQWGYIGSLSSNHDLYCSVHKGAH
VASSDAIMTRCLAVYDCFCNNINWNVEYPIISNELSINTSCRVLQRVILKAAMLCNRYTL
CYDIGNPKAIACVKDFDFKFYDAQPIVKSVKTLLYSFEAHKDSFKDGLCMFWNCNVDK
YPPNAVVCRFDTRVLNNLNLPGCNGGSLYVNKHAFHTKPFARAAFEHLKPMPFFYYSD
TPCVYMDGMDAKQVDYVPLKSATCITRCNLGGAVCLKHAEEYREYLESYNTATTAGFT
FWVYKTFDFYNLWNTFTKLQSLENVVYNLVKTGHYTGQAGEMPCAIINDKVVAKIDKE
DVVIFINNTTYPTNVAVELFAKRSVRHHPELKLFRNLNIDVCWKHVIWDYARESIFCSNT
YGVCMYTDLKFIDKLNVLFDGRDNGALEAFKRSNNGVYISTTKVKSLSMIRGPPRAELN
GVVVDKVGDTDCVFYFAVRKEGQDVIFSQFDSLGVSSNQSPQGNLGSNGKPGNVGGND
ALSISTIFTQSRVISSFTCRTDMEKDFIALDQDVFIQKYGLEDYAFEHIVYGNFNQKIIGGL
HLLIGLYRRQQTSNLVVQEFVSYDSSIHSYFITDEKSGGSKSVCTVIDILLDDFVALVKSL
NLNCVSKVVNVNVDFKDFQFMLWCNDEKVMTFYPRLQAASDWKPGYSMPVLYKYLN
SPMERVSLWNYGKPVTLPTGCMMNVAKYTQLCQYLNTTTLAVPVNMRVLHLGAGSE
KGVAPGSAVLRQWLPAGTILVDNDLYPFVSDSVATYFGDCITLPFDCQWDLIISDMYDPI
TKNIGEYNVSKDGFFTYICHMIRDKLALGGSVAIKITEFSWNAELYKLMGYFAFWTVFC
TNANASSSEGFLIGINYLCKPKVEIDGNVMHANYLFWRNSTVWNGGAYSLFDMAKFPL
KLAGTAVINLRADQINDMVYSLLEKGKLLIRDTNKEVFVGDSLVNVI (SEQ ID NO: 5)
NL63 (alpha coronavirus); NCBI GenBank accession code NC_005831:
MFYNQVTLAVASDSEISGFGFAIPSVAVRTYSEAAAQGFQACRFVAFGLQDCVTGINDD
DYVIALTGTNQLCAKILPFSDRPLNLRGWLIFSNSNYVLQDFDVVFGHGAGSVVFVDKY
MCGFDGKPVLPKNMWEFRDYFNNNTDSIVIGGVTYQLAWDVIRKDLSYEQQNVLAIESI
HYLGTTGHTLKSGCKLTNAKPPKYSSKVVLSGEWNAVYRAFGSPFITNGMSLLDIIVKP
VFFNAFVKCNCGSESWSVGAWDGYLSSCCGTPAKKLCVVPGNVVPGDVIITSTSAGCG
VKYYAGLVVKHITNITGVSLWRVTAVHSDGMFVASSSYDALLHRNSLDPFCFDVNTLL
SNQLRLAFLGASVTEDVKFAASTGVIDISAGMFGLYDDILTNNKPWFVRKASGLFDAIW
DAFVAAIKLVPTTTGVLVRFVKSIASTVLTVSNGVIIMCADVPDAFQSVYRTFTQAICAA
FDFSLDVFKIGDVKFKRLGDYVLTENALVRLTTEVVRGVRDARIKKAMFTKVVVGPTTE
VKFSVIELATVNLRLVDCAPVVCPKGKIVVIAGQAFFYSGGFYRFMVDPTTVLNDPVFT
GDLFYTIKFSGFKLDGFNHQFVTASSATDAIIAVELLLLDFKTAVFVYTCVVDGCSVIVR
RDATFATHVCFKDCYNVWEQFCIDNCGEPWFLTDYNAILQSNNPQCAIVQASESKVLLE
RFLPKCPEILLSIDDGHLWNLFVEKFNFVTDWLKTLKLTLTSNGLLGNCAKRFRRVLVK
LLDVYNGFLETVCSVAYTAGVCIKYYAVNVPYVVISGFVSRVIRRERCDMTFPCVSCVT
FFYEFLDTCFGVSKPNAIDVEHLELKETVFVEPKDGGQFFVSGDYLWYVVDDIYYPASC
NGVLPVAFTKLAGGKISFSDDVIVHDVEPTHKVKLIFEFEDDVVTSLCKKSFGKSIIYTGD
WEGLHEVLTSAMNVIGQHIKLPQFYIYDEEGGYDVSKPVMISQWPISNDSNGCVVEAST
DFHQLECIVDDSVREEVDIIEQPFEEVEHVLSIKQPFSFSFRDELGVRVLDQSDNNCWIST
TLVQLQLTKLLDDSIEMQLFKVGKVDSIVQKCYELSHLISGSLGDSGKLLSELLKEKYTC
SITFEMSCDCGKKFDDQVGCLFWIMPYTKLFQKGECCICHKMQTYKLVSMKGTGVFVQ
DPAPIDIDAFPVKPICSSVYLGVKGSGHYQTNLYSFNKAIDGFGVFDIKNSSVNTVCFVD
VDFHSVEIEAGEVKPFAVYKNVKFYLGDISHLVNCVSFDFVVNAANENLLHGGGVARAI
DILTEGQLQSLSKDYISSNGPLKVGAGVMLECEKFNVFNVVGPRTGKHEHSLLVEAYNS
ILFENGIPLMPLLSCGIFGVRIENSLKALFSCDINKPLQVFVYSSNEEQAVLKFLDGLDLTP
VIDDVDVVKPFRVEGNFSFFDCGVNALDGDIYLLFTNSILMLDKQGQLLDTKLNGILQQ
AALDYLATVKTVPAGNLVKLFVESCTIYMCVVPSINDLSFDKNLGRCVRKLNRLKTCVI
ANVPAIDVLKKLLSSLTLTVKFVVESNVMDVNDCFKNDNVVLKITEDGINVKDVVVESS
KSLGKQLGVVSDGVDSFEGVLPINTDTVLSVAPEVDWVAFYGFEKAALFASLDVKPYG
YPNDFVGGFRVLGTTDNNCWVNATCIILQYLKPTFKSKGLNVLWNKFVTGDVGPFVSFI
YFITMSSKGQKGDAEEALSKLSEYLISDSIVTLEQYSTCDICKSTVVEVKSAIVCASVLKD
GCDVGFCPHRHKLRSRVKFVNGRVVITNVGEPIISQPSKLLNGIAYTTFSGSFDNGHYVV
YDAANNAVYDGARLFSSDLSTLAVTAIVVVGGCVTSNVPTIVSEKISVMDKLDTGAQKF
FQFGDFVMNNIVLFLTWLLSMFSLLRTSIMKHDIKVIAKAPKRTGVILTRSFKYNIRSALF
VIKQKWCVIVTLFKFLLLLYAIYALVFMIVQFSPFNSLLCGDIVSGYEKSTFNKDIYCGNS
MVCKMCLFSYQEFNDLDHTSLVWKHIRDPILISLQPFVILVILLIFGNMYLRFGLLYFVAQ
FISTFGSFLGFHQKQWFLHFVPFDVLCNEFLATFIVCKIVLFVRHIIVGCNNADCVACSKS
ARLKRVPLQTIINGMHKSFYVNANGGTCFCNKHNFFCVNCDSFGPGNTFINGDIARELG
NVVKTAVQPTAPAYVIIDKVDFVNGFYRLYSGDTFWRYDFDITESKYSCKEVLKNCNVL
ENFIVYNNSGSNITQIKNACVYFSQLLCEPIKLVNSELLSTLSVDFNGVLHKAYVDVLCN
SFFKELTANMSMAECKATLGLTVSDDDFVSAVANAHRYDVLLSDLSFNNFFISYAKPED
KLSVYDIACCMRAGSKVVNHNVLIKESIPIVWGVKDFNTLSQEGKKYLVKTTKAKGLTF
LLTFNDNQAITQVPATSIVAKQGAGFKRTYNFLWYVCLFVVALFIGVSFIDYTTTVTSFH
GYDFKYIENGQLKVFEAPLHCVRNVFDNFNQWHEAKFGVVTTNSDKCPIVVGVSERIN
VVPGVPTNVYLVGKTLVFTLQAAFGNTGVCYDFDGVTTSDKCIFNSACTRLEGLGGDN
VYCYNTDLIEGSKPYSTLQPNAYYKYDAKNYVRFPEILARGFGLRTIRTLATRYCRVGE
CRDSHKGVCFGFDKWYVNDGRVDDGYICGDGLIDLLVNVLSIFSSSFSVVAMSGHMLF
NFLFAAFITFLCFLVTKFKRVFGDLSYGVFTVVCATLINNISYVVTQNLFFMLLYAILYFV
FTRTVRYAWIWHIAYIVAYFLLIPWWLLTWFSFAAFLELLPNVFKLKISTQLFEGDKFIGT
FESAAAGTFVLDMRSYERLINTISPEKLKNYAASYNKYKYYSGSASEADYRCACYAHLA
KAMLDYAKDHNDMLYSPPTISYNSTLQSGLKKMAQPSGCVERCVVRVCYGSTVLNGV
WLGDTVTCPRHVIAPSTTVLIDYDHAYSTMRLHNFSVSHNGVFLGVVGVTMHGSVLRI
KVSQSNVHTPKHVFKTLKPGDSFNILACYEGIASGVFGVNLRTNFTIKGSFINGACGSPG
YNVRNDGTVEFCYLHQIELGSGAHVGSDFTGSVYGNFDDQPSLQVESANLMLSDNVVA
FLYAALLNGCRWWLCSTRVNVDGFNEWAMANGYTSVSSVECYSILAAKTGVSVEQLL
ASIQHLHEGFGGKNILGYSSLCDEFTLAEVVKQMYGVNLQSGKVIFGLKTMFLFSVFFT
MFWAELFIYTNTIWINPVILTPIFCLLLFLSLVLTMFLKHKFLFLQVFLLPTVIATALYNCV
LDYYIVKFLADHFNYNVSVLQMDVQGLVNVLVCLFVVFLHTWRFSKERFTHWFTYVC
SLIAVAYTYFYSGDFLSLLVMFLCAISSDWYIGAIVFRLSRLIVFFSPESVFSVFGDVKLTL
VVYLICGYLVCTYWGILYWFNRFFKCTMGVYDFKVSAAEFKYMVANGLHAPHGPFDA
LWLSFKLLGIGGDRCIKISTVQSKLTDLKCTNVVLLGCLSSMNIAANSSEWAYCVDLHN
KINLCDDPEKAQSMLLALLAFFLSKHSDFGLDGLIDSYFDNSSTLQSVASSFVSMPSYIAY
ENARQAYEDAIANGSSSQLIKQLKRAMNIAKSEFDHEISVQKKINRMAEQAATQMYKEA
RSVNRKSKVISAMHSLLFGMLRRLDMSSVETVLNLARDGVVPLSVIPATSASKLTIVSPD
LESYSKIVCDGSVHYAGVVWTLNDVKDNDGRPVHVKEITKENVETLTWPLILNCERVV
KLQNNEIMPGKLKQKPMKAEGDGGVLGDGNALYNTEGGKTFMYAYISNKADLKFVK
WEYEGGCNTIELDSPCRFMVETPNGPQVKYLYFVKNLNTLRRGAVLGFIGATIRLQAGK
QTELAVNSGLLTACAFSVDPATTYLEAVKHGAKPVSNCIKMLSNGAGNGQAITTSVDA
NTNQDSYGGASICLYCRAHVPHPSMDGYCKFKGKCVQVPIGCLDPIRFCLENNVCNVC
GCWLGHGCACDRTTIQSVDISYLNRARGSSAARLEPCNGTDIDKCVRAFDIYNKNVSFL
GKCLKMNCVRFKNADLKDGYFVIKRCTKSVMEHEQSMYNLLNFSGALAEHDFFTWKD
GRVIYGNVSRHNLTKYTMMDLVYAMRNFDEQNCDVLKEVLVLTGCCDNSYFDSKGW
YDPVENEDIHRVYASLGKIVARAMLKCVALCDAMVAKGVVGVLTLDNQDLNGNFYDF
GDFVVSLPNMGVPCCTSYYSYMMPEVIGLTNCLASECFVKSDIFGSDFKTFDLLKYDFTE
HKENLFNKYFKHWSFDYHPNCSDCYDDMCVIHCANFNTLFATTIPGTAFGPLCRKVFID
GVPLVTTAGYHFKQLGLVWNKDVNTHSVRLTITELLQFVTDPSLIIASSPALVDQRTICFS
VAALSTGLTNQVVKPGHFNEEFYNFLRLRGFFDEGSELTLKHFFFAQNGDAAVKDFDFY
RYNKPTILDICQARVTYKIVSRYFDIYEGGCIKACEVVVTNLNKSAGWPLNKFGKASLY
YESISYEEQDALFALTKRNVLPTMTQLNLKYAISGKERARTVGGVSLLSTMTTRQYHQK
HLKSIVNTRNATVVIGTTKFYGGWNNMLRTLIDGVENPMLMGWDYPKCDRALPNMIR
MISAMVLGSKHVNCCTATDRFYRLGNELAQVLTEVVYSNGGFYFKPGGTTSGDASTAY
ANSIFNIFQAVSSNINRLLSVPSDSCNNVNVRDLQRRLYDNCYRLTSVEESFIDDYYGYL
RKHFSMMILSDDGVVCYNKDYAELGYIADISAFKATLYYQNNVFMSTSKCWVEEDLTK
GPHEFCSQHTMQIVDKDGTYYLPYPDPSRILSAGVFVDDVVKTDAVVLLERYVSLAIDA
YPLSKHPNSEYRKVFYVLLDWVKHLNKNLNEGVLESFSVTLLDNQEDKFWCEDFYAS
MYENSTILQAAGLCVVCGSQTVLRCGDCLRKPMLCTKCAYDHVFGTDHKFILAITPYVC
NASGCGVSDVKKLYLGGLNYYCTNHKPQLSFPLCSAGNIFGLYKNSATGSLDVEVFNRL
ATSDWTDVRDYKLANDVKDTLRLFAAETIKAKEESVKSSYAFATLKEVVGPKELLLSW
ESGKVKPPLNRNSVFTCFQISKDSKFQIGEFIFEKVEYGSDTVTYKSTVTTKLVPGMIFVL
TSHNVQPLRAPTIANQEKYSSIYKLHPAFNVSDAYANLVPYYQLIGKQKITTIQGPPGSG
KSHCSIGLGLYYPGARIVFVACAHAAVDSLCAKAMTVYSIDKCTRIIPARARVECYSGFK
PNNTSAQYIFSTVNALPECNADIVVVDEVSMCTNYDLSVINQRLSYKHIVYVGDPQQLP
APRVMITKGVMEPVDYNVVTQRMCAIGPDVFLHKCYRCPAEIVNTVSELVYENKFVPV
KPASKQCFKVFFKGNVQVDNGSSINRKQLEIVKLFLVKNPSWSKAVFISPYNSQNYVAS
RFLGLQIQTVDSSQGSEYDYVIYAQTSDTAHACNVNRFNVAITRAKKGIFCVMCDKTLF
DSLKFFEIKHADLHSSQVCGLFKNCTRTPLNLPPTHAHTFLSLSDQFKTTGDLAVQIGSN
NVCTYEHVISFMGFRFDISIPGSHSLFCTRDFAIRNVRGWLGMDVESAHVCGDNIGTNVP
LQVGFSNGVNFVVQTEGCVSTNFGDVIKPVCAKSPPGEQFRHLIPLLRKGQPWLIVRRRI
VQMISDYLSNLSDILVFVLWAGSLELTTMRYFVKIGPIKYCYCGNSATCYNSVSNEYCCF
KHALGCDYVYNPYAFDIQQWGYVGSLSQNHHTFCNIHRNEHDASGDAVMTRCLAVHD
CFVKNVDWTVTYPFIANEKFINGCGRNVQGHVVRAALKLYKPSVIHDIGNPKGVRCAV
TDAKWYCYDKQPVNSNVKLLDYDYATHGQLDGLCLFWNCNVDMYPEFSIVCRFDTRT
RSVFNLEGVNGGSLYVNKHAFHTPAYDKRAFVKLKPMPFFYFDDSDCDVVQEQVNYV
PLRASSCVTRCNIGGAVCSKHANLYQKYVEAYNTFTQAGFNIWVPHSFDVYNLWQIFIE
TNLQSLENIAFNVVKKGCFTGVDGELPVAVVNDKVFVRYGDVDNLVFTNKTTLPTNVA
FELFAKRKMGLTPPLSILKNLGVVATYKFVLWDYEAERPFTSYTKSVCKYTDFNEDVCV
CFDNSIQGSYERFTLTTNAVLFSTVVIKNLTPIKLNFGMLNGMPVSSIKGDKGVEKLVNW
YIYVRKNGQFQDHYDGFYTQGRNLSDFTPRSDMEYDFLNMDMGVFINKYGLEDFNFEH
VVYGDVSKTTLGGLHLLISQFRLSKMGVLKADDFVTASDTTLRCCTVTYLNELSSKVVC
TYMDLLLDDFVTILKSLDLGVISKVHEVIIDNKPYRWMLWCKDNHLSTFYPQLQSAEW
KCGYAMPQIYKLQRMCLEPCNLYNYGAGIKLPSGIMLNVVKYTQLCQYLNSTTMCVPH
NIVIRVLHYGAGSDKGVAPGTTVLKRWLPPDAIIIDNDINDYVSDADFSITGDCATVYLED
KFDLLISDMYDGRIKFCDGENVSKDGFFTYLNGVIREKLAIGGSVAIKITEYSWNKYLYE
LIQRFAFWTLFCTSVNTSSSEAFLIGINYLGDFIQGPFIAGNTVHANYIFWRNSTIMSLSYN
SVLDLSKFECKHKATVVVTLKDSDVNDMVLSLIKSGRLLLRNNGRFGGFSNHLVSTK
(SEQ ID NO: 6)
229E (alpha coronavirus); NCBI GenBank accession code NC_002645:
MACNRVTLAVASDSEISANGCSTIAQAVRRYSEAASNGFRACRFVSLDLQDCIVGIADD
TYVMGLHGNQTLFCNIMKFSDRPFMLHGWLVFSNSNYLLEEFDVVFGKRGGGNVTYT
DQYLCGADGKPVMSEDLWQFVDHFGENEEIIINGHTYVCAWLTKRKPLDYKRQNNLAI
EEIEYVHGDALHTLRNGSVLEMAKEVKTSSKVVLSDALDKLYKVFGSPVMTNGSNILEA
FTKPVFISALVQCTCGTKSWSVGDWTGFKSSCCNVISNKLCVVPGNVKPGDAVITTQQA
GAGIKYFCGMTLKFVANIEGVSVWRVIALQSVDCFVASSTFVEEEHVNRMDTFCFNVR
NSVTDECRLAMLGAEMTSNVRRQVASGVIDISTGWFDVYDDIFAESKPWFVRKAEDIFG
PCWSALASALKQLKVTTGELVRFVKSICNSAVAVVGGTIQILASVPEKFLNAFDVFVTAI
QTVFDCAVETCTIAGKAFDKVFDYVLLDNALVKLVTTKLKGVRERGLNKVKYATVVV
GSTEEVKSSRVERSTAVLTIANNYSKLFDEGYTVVIGDVAYFVSDGYFRLMASPNSVLT
TAVYKPLFAFNVNVMGTRPEKFPTTVTCENLESAVLFVNDKITEFQLDYSIDVIDNEIIVK
PNISLCVPLYVRDYVDKWDDFCRQYSNESWFEDDYRAFISVLDITDAAVKAAESKAFV
DTIVPPCPSILKVIDGGKIWNGVIKNVNSVRDWLKSLKLNLTQQGLLGTCAKRFKRWLGI
LLEAYNAFLDTVVSTVKIGGLTFKTYAFDKPYIVIRDIVCKVENKTEAEWIELFPHNDRIK
SFSTFESAYMPIADPTHFDIEEVELLDAEFVEPGCGGILAVIDEHVFYKKDGVYYPSNGT
NILPVAFTKAAGGKVSFSDDVEVKDIEPVYRVKLCFEFEDEKLVDVCEKAIGKKIKHEG
DWDSFCKTIQSALSVVSCYVNLPTYYIYDEEGGNDLSLPVMISEWPLSVQQAQQEATLP
DIAEDVVDQVEEVNSIFDIETVDVKHDVSPFEMPFEELNGLKILKQLDNNCWVNSVMLQ
IQLTGILDGDYAMQFFKMGRVAKMIERCYTAEQCIRGAMGDVGLCMYRLLKDLHTGF
MVMDYKCSCTSGRLEESGAVLFCTPTKKAFPYGTCLNCNAPRMCTIRQLQGTIIFVQQK
PEPVNPVSFVVKPVCSSIFRGAVSCGHYQTNIYSQNLCVDGFGVNKIQPWTNDALNTICI
KDADYNAKVEISVTPIKNTVDTTPKEEFVVKEKLNAFLVHDNVAFYQGDVDTVVNGVD
FDFIVNAANENLAHGGGLAKALDVYTKGKLQRLSKEHIGLAGKVKVGTGVMVECDSL
RIFNVVGPRKGKHERDLLIKAYNTINNEQGTPLTPILSCGIFGIKLETSLEVLLDVCNTKEV
KVFVYTDTEVCKVKDFVSGLVNVQKVEQPKIEPKPVSVIKVAPKPYRVDGKFSYFTEDL
LCVADDKPIVLFTDSMLTLDDRGLALDNALSGVLSAAIKDCVDINKAIPSGNLIKFDIGSV
VVYMCVVPSEKDKHLDNNVQRCTRKLNRLMCDIVCTIPADYILPLVLSSLTCNVSFVGE
LKAAEAKVITIKVTEDGVNVHDVTVTTDKSFEQQVGVIADKDKDLSGAVPSDLNTSELL
TKAIDVDWVEFYGFKDAVTFATVDHSAFAYESAVVNGIRVLKTSDNNCWVNAVCIALQ
YSKPHFISQGLDAAWNKFVLGDVEIFVAFVYYVARLMKGDKGDAEDTLTKLSKYLANE
AQVQLEHYSSCVECDAKFKNSVASINSAIVCASVKRDGVQVGYCVHGIKYYSRVRSVR
GRAIIVSVEQLEPCAQSRLLSGVAYTAFSGPVDKGHYTVYDTAKKSMYDGDRFVKHDL
SLLSVTSVVMVGGYVAPVNTVKPKPVINQLDEKAQKFFDFGDFLIHNFVIFFTWLLSMF
TLCKTAVTTGDVKIMAKAPQRTGVVLKRSLKYNLKASAAVLKSKWWLLAKFTKLLLLI
YTLYSVVLLCVRFGPFNFCSETVNGYAKSNFVKDDYCDGSLGCKMCLFGYQELSQFSH
LDVVWKHITDPLFSNMQPFIVMVLLLIFGDNYLRCFLLYFVAQMISTVGVFLGYKETNW
FLHFIPFDVICDELLVTVIVIKVISFVRHVLFGCENPDCIACSKSARLKRFPVNTIVNGVQR
SFYVNANGGSKFCKKHRFFCVDCDSYGYGSTFITPEVSRELGNITKTNVQPTGPAYVMI
DKVEFENGFYRLYSCETFWRYNFDITESKYSCKEVFKNCNVLDDFIVFNNNGTNVTQVK
NASVYFSQLLCRPIKLVDSELLSTLSVDFNGVLHKAYIDVLRNSFGKDLNANMSLAECK
RALGLSISDHEFTSAISNAHRCDVLLSDLSFNNFVSSYAKPEEKLSAYDLACCMRAGAK
VVNANVLTKDQTPIVWHAKDFNSLSAEGRKYIVKTSKAKGLTFLLTINENQAVTQIPAT
SIVAKQGAGDAGHSLTWLWLLCGLVCLIQFYLCFFMPYFMYDIVSSFEGYDFKYIENGQ
LKNFEAPLKCVRNVFENFEDWHYAKFGFTPLNKQSCPIVVGVSEIVNTVAGIPSNVYLV
GKTLIFTLQAAFGNAGVCYDIFGVTTPEKCIFTSACTRLEGLGGNNVYCYNTALMEGSLP
YSSIQANAYYKYDNGNFIKLPEVIAQGFGFRTVRTIATKYCRVGECVESNAGVCFGFDK
WFVNDGRVANGYVCGTGLWNLVFNILSMFSSSFSVAAMSGQILLNCALGAFAIFCCFLV
TKFRRMFGDLSVGVCTVVVAVLLNNVSYIVTQNLVTMIAYAILYFFATRSLRYAWIWC
AAYLIAYISFAPWWLCAWYFLAMLTGLLPSLLKLKVSTNLFEGDKFVGTFESAAAGTFV
IDMRSYEKLANSISPEKLKSYAASYNRYKYYSGNANEADYRCACYAYLAKAMLDFSRD
HNDILYTPPTVSYGSTLQAGLRKMAQPSGFVEKCVVRVCYGNTVLNGLWLGDIVYCPR
HVIASNTTSAIDYDHEYSIMRLHNFSIISGTAFLGVVGATMHGVTLKIKVSQTNMHTPRH
SFRTLKSGEGFNILACYDGCAQGVFGVNMRTNWTIRGSFINGACGSPGYNLKNGEVEFV
YMHQIELGSGSHVGSSFDGVMYGGFEDQPNLQVESANQMLTVNVVAFLYAAILNGCT
WWLKGEKLFVEHYNEWAQANGFTAMNGEDAFSILAAKTGVCVERLLHAIQVLNNGFG
GKQILGYSSLNDEFSINEVVKQMFGVNLQSGKTTSMFKSISLFAGFFVMFWAELFVYTTT
IWVNPGFLTPFMILLVALSLCLTFVVKHKVLFLQVFLLPSIIVAAIQNCAWDYHVTKVLA
EKFDYNVSVMQMDIQGFVNIFICLFVALLHTWRFAKERCTHWCTYLFSLIAVLYTALYS
YDYVSLLVMLLCAISNEWYIGAIIFRICRFGVAFLPVEYVSYFDGVKTVLLFYMLLGFVS
CMYYGLLYWINRFCKCTLGVYDFCVSPAEFKYMVANGLNAPNGPFDALFLSFKLMGIG
GPRTIKVSTVQSKLTDLKCTNVVLMGILSNMNIASNSKEWAYCVEMHNKINLCDDPETA
QELLLALLAFFLSKHSDFGLGDLVDSYFENDSILQSVASSFVGMPSFVAYETARQEYENA
VANGSSPQIIKQLKKAMNVAKAEFDRESSVQKKINRMAEQAAAAMYKEARAVNRKSK
VVSAMHSLLFGMLRRLDMSSVDTILNMARNGVVPLSVIPATSAARLVVVVPDHDSFVK
MMVDGFVHYAGVVWTLQEVKDNDGKNVHLKDVTKENQEILVWPLILTCERVVKLQN
NEIMPGKMKVKATKGEGDGGITSEGNALYNNEGGRAFMYAYVTTKPGMKYVKWEHD
SGVVTVELEPPCRFVIDTPTGPQIKYLYFVKNLNNLRRGAVLGYIGATVRLQAGKQTEFV
SNSHLLTHCSFAVDPAAAYLDAVKQGAKPVGNCVKMLTNGSGSGQAITCTIDSNTTQD
TYGGASVCIYCRAHVAHPTMDGFCQYKGKWVQVPIGTNDPIRFCLENTVCKVCGCWL
NHGCTCDRTAIQSFDNSYLNRVRGSSAARLEPCNGTDIDYCVRAFDVYNKDASFIGKNL
KSNCVRFKNVDKDDAFYIVKRCIKSVMDHEQSMYNLLKGCNAVAKHDFFTWHEGRTI
YGNVSRQDLTKYTMMDLCFALRNFDEKDCEVFKEILVLTGCCSTDYFEMKNWFDPIEN
EDIHRVYAALGKVVANAMLKCVAFCDEMVLKGVVGVLTLDNQDLNGNFYDFGDFVL
CPPGMGIPYCTSYYSYMMPVMGMTNCLASECFMKSDIFGQDFKTFDLLKYDFTEHKEV
LFNKYFKYWGQDYHPDCVDCHDEMCILHCSNFNTLFATTIPNTAFGPLCRKVFIDGVPV
VATAGYHFKQLGLVWNKDVNTHSTRLTITELLQFVTDPTLIVASSPALVDKRTVCFSVA
ALSTGLTSQTVKPGHFNKEFYDFLRSQGFFDEGSELTLKHFFFTQKGDAAIKDFDYYRY
NRPTMLDIGQARVAYQVAARYFDCYEGGCITSREVVVTNLNKSAGWPLNKFGKAGLY
YESISYEEQDAIFSLTKRNILPTMTQLNLKYAISGKERARTVGGVSLLATMTTRQFHQKC
LKSIVATRNATVVIGTTKFYGGWDNMLKNLMADVDDPKLMGWDYPKCDRAMPSMIR
MLSAMILGSKHVTCCTASDKFYRLSNELAQVLTEVVYSNGGFYFKPGGTTSGDATTAY
ANSVFNIFQAVSSNINCVLSVNSSNCNNFNVKKLQRQLYDNCYRNSNVDESFVDDFYGY
LQKHFSMMILSDDSVVCYNKTYAGLGYIADISAFKATLYYQNGVFMSTAKCWTEEDLSI
GPHEFCSQHTMQIVDENGKYYLPYPDPSRIISAGVFVDDITKTDAVILLERYVSLAIDAYP
LSKHPKPEYRKVFYALLDWVKHLNKTLNEGVLESFSVTLLDEHESKFWDESFYASMYE
KSTVLQAAGLCVVCGSQTVLRCGDCLRRPMLCTKCAYDHVFGTDHKFILAITPYVCNTS
GCNVNDVTKLYLGGLNYYCVDHKPHLSFPLCSAGNVFGLYKSSALGSMDIDVFNKLST
SDWSDIRDYKLANDAKESLRLFAAETVKAKEESVKSSYAYATLKEIVGPKELLLLWESG
KAKPPLNRNSVFTCFQITKDSKFQVGEFVFEKVDYGSDTVTYKSTATTKLVPGMLFILTS
HNVAPLRAPTMANQEKYSTIYKLHPSFNVSDAYANLVPYYQLIGKQRITTIQGPPGSGKS
HCSIGIGVYYPGARIVFTACSHAAVDSLCAKAVTAYSVDKCTRIIPARARVECYSGFKPN
NNSAQYVFSTVNALPEVNADIVVVDEVSMCTNYDLSVINQRISYKHIVYVGDPQQLPAP
RVLISKGVMEPIDYNVVTQRMCAIGPDVFLHKCYRCPAEIVNTVSELVYENKFVPVKEA
SKQCFKIFERGSVQVDNGSSINRRQLDVVKRFIHKNSTWSKAVFISPYNSQNYVAARLLG
LQTQTVDSAQGSEYDYVIFAQTSDTAHACNANRFNVAITRAKKGIFCIMSDRTLFDALK
FFEITMTDLQSESSCGLFKDCARNPIDLPPSHATTYLSLSDRFKTSGDLAVQIGNNNVCTY
EHVISYMGFRFDVSMPGSHSLFCTRDFAMRHVRGWLGMDVEGAHVTGDNVGTNVPLQ
VGFSNGVDFVAQPEGCVLTNTGSVVKPVRARAPPGEQFTHIVPLLRKGQPWSVLRKRIV
QMIADFLAGSSDVLVFVLWAGGLELTTMRYFVKIGAVKHCQCGTVATCYNSVSNDYC
CFKHALGCDYVYNPYVIDIQQWGYVGSLSTNHHAICNVHRNEHVASGDAIMTRCLAVY
DCFVKNVDWSITYPMIANENAINKGGRTVQSHIMRAAIKLYNPKAIHDIGNPKGIRCAVT
DAKWYCYDKNPINSNVKTLEYDYMTHGQMDGLCLFWNCNVDMYPEFSIVCRFDTRTR
STLNLEGVNGGSLYVNNHAFHTPAYDKRAMAKLKPAPFFYYDDGSCEVVHDQVNYVP
LRATNCITKCNIGGAVCSKHANLYRAYVESYNIFTQAGFNIWVPTTFDCYNLWQTFTEV
NLQGLENIAFNVVNKGSFVGADGELPVAISGDKVFVRDGNTDNLVFVNKTSLPTNIAFE
LFAKRKVGLTPPLSILKNLGVVATYKFVLWDYEAERPLTSFTKSVCGYTDFAEDVCTCY
DNSIQGSYERFTLSTNAVLFSATAVKTGGKSLPAIKLNFGMLNGNAIATVKSEDGNIKNI
NWFVYVRKDGKPVDHYDGFYTQGRNLQDFLPRSTMEEDFLNMDIGVFIQKYGLEDFNF
EHVVYGDVSKTTLGGLHLLISQVRLSKMGILKAEEFVAASDITLKCCTVTYLNDPSSKTV
CTYMDLLLDDFVSVLKSLDLTVVSKVHEVIIDNKPWRWMLWCKDNAVATFYPQLQSA
EWKCGYSMPGIYKTQRMCLEPCNLYNYGAGLKLPSGIMFNVVKYTQLCQYFNSTTLCV
PHNMRVLHLGAGSDYGVAPGTAVLKRWLPHDAIVVDNDVVDYVSDADFSVTGDCAT
VYLEDKFDLLISDMYDGRTKAIDGENVSKEGFFTYINGFICEKLAIGGSIAIKVTEYSWNK
KLYELVQRFSFWTMFCTSVNTSSSEAFVVGINYLGDFAQGPFIDGNIIHANYVFWRNSTV
MSLSYNSVLDLSKFNCKHKATVVVQLKDSDINEMVLSLVRSGKLLVRGNGKCLSFSNH
LVSTK (SEQ ID NO: 7)

What is claimed:

1. A method of producing an immune globulin comprising:
1) obtaining a plurality of plasma samples from a plurality of plasma donors;
2) conducting a first assay on each plasma sample to measure total anti-SARS CoV-2 antibody titer;
3) selecting, based upon the first assay, plasma samples having a total anti-SARS CoV-2 antibody binding titer that is at least two-fold higher than the amount of total anti-SARS CoV-2 antibody binding titer in a control sample;
4) conducting a second assay on each selected plasma sample from step (3) to measure SARS CoV-2 neutralizing antibody titer;
5) identifying, based upon the second assay, plasma samples having a neutralizing antibody titer in the lower 65% of all plasma samples assayed and excluding the identified plasma samples from further processing;
6) pooling the non-excluded plasma samples from step (5); and
7) preparing immune globulin from the pooled plasma samples of step (6).

2. The method of claim 1, wherein each of the plurality of plasma donors is a COVID-19 convalescent plasma donor.

3. The method of claim 1, wherein each of the plurality of plasma donors is a COVID-19 vaccinated plasma donor.

4. The method of claim 1, wherein the control sample is a mixture of plasma samples obtained from 100 or more random human plasma donors.

5. The method of claim 1, wherein the control sample is a commercially available immune globulin.

6. The method of claim 1, wherein step (5) comprises identifying, based upon the second assay, plasma samples having a neutralizing antibody titer in the lower 70% of all plasma samples assayed and excluding the identified plasma samples from further processing.

7. The method of claim 1, wherein step (5) comprises identifying, based upon the second assay, plasma samples having a neutralizing antibody titer in the lower 75% of all plasma samples assayed and excluding the identified plasma samples from further processing.

8. The method of claim 1, wherein the number of non-excluded, pooled plasma samples is 250 or more.

9. The method of claim 1, wherein the number of non-excluded, pooled plasma samples is 500 or more.

10. The method of claim 1, wherein the immune globulin is prepared using a cold alcohol fractionation process that isolates the immune globulin fraction from the pooled plasma as a solution.

11. The method of claim 1, wherein the immune globulin is combined with a pharmaceutically acceptable carrier.

12. A method of providing immunotherapy to a subject in need thereof, comprising administering to the subject an immunotherapeutic composition comprising:
A) an immune globulin prepared from pooled plasma samples, wherein the pooled plasma samples are obtained by:
1) obtaining a plurality of plasma samples from a plurality of plasma donors;
2) conducting a first assay on each plasma sample to measure total anti-SARS CoV-2 antibody titer;
3) selecting, based upon the first assay, plasma samples having a total anti-SARS CoV-2 antibody binding titer that is at least two-fold higher than the amount of total anti-SARS CoV-2 antibody binding titer in a control sample, wherein the control sample is a mixture of plasma samples obtained from 100 or more random human plasma donors;
4) conducting a second assay on each selected plasma sample from step (3) to measure SARS CoV-2 neutralizing antibody titer;
5) identifying, based upon the second assay, plasma samples having a neutralizing antibody titer in the lower 65% of all plasma samples assayed and excluding the identified plasma samples from further processing; and
6) pooling the non-excluded plasma samples from step (5) to generate the pooled plasma samples; and
B) a pharmaceutically acceptable carrier;
wherein the immunotherapeutic composition is administered to the subject so as to provide from about 1.5-2.0 grams of immune globulin per kilogram of the subject.

13. The method of claim 12, wherein each of the plurality of plasma donors is a COVID-19 convalescent plasma donor.

14. The method of claim 12, wherein each of the plurality of plasma donors is a COVID-19 vaccinated plasma donor.

15. The method of claim 12, wherein the immunotherapeutic composition further comprises a biologically active agent selected from the group consisting of an anti-inflammatory agent, an anti-cancer agent, an anti-microbial agent, an antihistamine, a cytokine, and a chemokine.

16. The method of claim 12, wherein the immunotherapeutic composition further comprises an immunotherapeutic agent selected from the group consisting of a recombinant antibody, an antibody fragment, an antibody-like molecule, a monoclonal antibody, an antiviral, an immunotherapeutic protein and an immunotherapeutic small molecule.

17. The method of claim 12, wherein the immunotherapeutic composition further comprises an anti-inflammatory agent selected from the group consisting of a recombinant antibody, an antibody fragment, a monoclonal antibody, an anti-inflammatory protein and an anti-inflammatory small molecule.

18. The method of claim 12, wherein the subject is diagnosed with COVID-19.

19. The method of claim 12, wherein the subject is age 65 or older.

20. A pharmaceutical composition comprising an immune globulin obtained by the method of claim 1.
